SEQUENCE LISTING (10 sequences)

SEQ ID NO: 1 - DNA, 18 bp, synthetic construct
  source 1..18: mol_type = other DNA
ggtgacgatcccgcaaaa

SEQ ID NO: 2 - DNA, 37 bp, synthetic construct
  source 1..37: mol_type = other DNA
tgcgcatccattgtagaggtggtcgctgaggcttgca

SEQ ID NO: 3 - DNA, 37 bp, synthetic construct
  source 1..37: mol_type = other DNA
acctctacaatggatgcgcaggtcgctgaggcttgca

SEQ ID NO: 4 - DNA, 22 bp, synthetic construct
  source 1..22: mol_type = other DNA
agtaacaaccgatcccgcaaaa

SEQ ID NO: 5 - DNA, 14 bp, synthetic construct
  source 1..14: mol_type = other DNA
cggcctttaactcc

SEQ ID NO: 6 - DNA, 14 bp, synthetic construct
  source 1..14: mol_type = other DNA
acctaccggcctcc

SEQ ID NO: 7 - DNA, 66 bp, synthetic construct
  source 1..66: mol_type = other DNA
tttttggtgacgatcccgcaaaatnnnnnnnnnncacctaccggcctcctgcaagcctcagcgacc

SEQ ID NO: 8 - DNA, 56 bp, synthetic construct
  source 1..56: mol_type = other DNA
tttttggtgacgatcccgcaaaatcacctaccggcctcctgcaagcctcagcgacc

SEQ ID NO: 9 - DNA, 66 bp, synthetic construct
  source 1..66: mol_type = other DNA
tttttggtgacgatcccgcaaaagnnnnnnnnnncggcctttaactccctgcaagcctcagcgacc

SEQ ID NO: 10 - DNA, 15 bp, synthetic construct
  source 1..15: mol_type = other DNA
ggttggtgtggttgg

(19) United States
(12) Patent Application Publication
(10) Pub. No.: US 2026/0258474 A1
(43) Pub. Date: Sep. 3, 2026

(54) DETECTION OF BIOMOLECULES IN SINGLE CELLS

(71) Applicant: Actome GmbH, Freiburg (DE)

(72) Inventor: Csaba Jeney, Müllheim (DE)

(21) Appl. No.: 18/879,644

(22) PCT Filed: Jun. 28, 2023
(86) PCT No.: PCT/EP2023/067598

§ 371 Date: Dec. 27, 2024

(30) Foreign Application Priority Data
Jun. 29, 2022 (EP) 22181760.4

(51) Int. Cl. C12Q 1/6804; C12Q 1/6806; G01N 33/575; G01N 33/68

(52) U.S. Cl. CPC C12Q 1/6804; C12Q 1/6806; G01N 33/5759; G01N 33/6827; G01N 2458/10

(57) ABSTRACT

A method for determining the concentration of at least one target analyte in a sample comprising: a. providing at least a binding agent of a first type and a binding agent of a second type, each comprising a label that is unique for the type of binding agent, wherein the binding agent of a first type and the binding agent of a second type specifically bind to a first target analyte, b. determining the binding characteristics of the binding agent of a first type and the binding agent of a second type to the first target analyte, c. providing a sample comprising cells at a known concentration, d. bringing the binding agent of a first type and the binding agent of a second type into contact with the sample, wherein the binding agent of a first type and the binding agent of a second type bind to the first target analyte forming an analyte-binding agent complex (couplexes), e. compartmentalizing single cells of the sample into a plurality of first partitions, f. complementing each first partition comprising a single cell with a lysis buffer, thereby lysing the single cell in each of the first partitions, g. compartmentalizing single analyte-binding agent complexes (couplexes) into a plurality of second partitions, h. performing a bi-component detection method, thereby determining the number and/or absolute concentration of couplexes and binding agents in the second partition, and i. determining the absolute concentration of the first target analyte per cell by taking into account the initial concentration of the cells in the sample in step a., the binding characteristics determined in step b. and the number and/or absolute concentration of couplexes and binding agents determined in step h.

### FIELD OF THE INVENTION

The present invention relates generally to the field of molecular biology. More particularly, it concerns methods for detecting targets including proteins, post translationally modified proteins and protein interactions in single-cells. The present invention provides such methods with high sensitivity having a detection limit of preferably below 100 or preferably 10 targets per cell and in absolute molecular quantities.

### BACKGROUND OF THE INVENTION

Proteomics encompasses proteomic domains, which are equally important to study proteomic functions, including expression proteomics concerning the changes in protein expression levels under different physiological or diseased cellular regimes, pathway organizations determined by protein-protein interactions, enzyme-substrate relationships and posttranslational modifications of the proteins and structural proteomics predicting the three-dimensional structure and assembly of higher order structures of proteins and protein complexes (complexomics). However, to provide a direct link to the protein's role in the physiological context of the cell quantitative information of all of above also needs to be assessed, measuring systematic perturbation or functional inactivation of proteins within its physiological environment to address the potential role of the target protein in a cellular process, akin to gene disruption to elucidate the function of a gene. The parallel evaluation of quantitative protein functions at the cellular and molecular level allows the necessary integrative view to understand the information processing nature of the cellular pathways that ultimately determines the workings of the cell.

In case of single-cells, the behavior of individual cells is governed by the principle that a cell's workings, developmental trajectory and responsiveness is heterogeneous even if the cells are originally from the same ancestry. A cell, for instance, has a certain chance of being in one transcriptional state and another chance of being at another transcriptional state, and cells are traversing between states governed only partly by known stimuli. When cells interact with the environment, purely as a consequence of these effects, a host of odd phenotypes should ensue. But observing such cellar behavior amid the overwhelming noise and unsatisfactory analytical power including low sensitivity of measurements is a daunting undertaking necessitating new single-cell analytical methods.

Existing single/cell analytical methods are only partially designed to solve this conundrum and defined by collecting comprehensive, information processing layers of cells through related chemical information of the individual members of large cell populations, including genomic, transcriptomic, epigenomic methods. However, while proteomic methods are also developed these are limited in scope compared to the more developed DNA- and RNA-based methods of single-cell assays. Nevertheless, no methods are available for single-cell interactomics, epi-proteomics (for the sake of this document the antibody distinguishable modified proteins including post-translationally modified proteins are referred as epiproteins) and complexomics.

Regarding the sensitivity of proteomics, interactomics, epi-proteomics and complexomic single-cell methods, a sensitivity of 100 s or preferably 10 s of proteins in absolute molecular amounts is required to fully grasp at the functional significance of the detected amounts. To reach these limits is not possible with contemporary single-cell assays especially for interactomics, epi-proteomics and complexomic.

The currently used methods are aimed at capturing the genome, e.g., MDA-WGA, isothermal amplification (Spits, C. et al. Whole-genome multiple displacement amplification from single cells. Nat. Protoc. 1, 1965-1970 2006), DOP-PCR-WGA, PCR-based (Telenius, H. et al. Degenerate oligonucleotide-primed PCR: general amplification of target DNA by a single degenerate primer. Genomics 13, 718-725 1992), MALBAC-WGA (Zong, C., Lu, S., Chapman, A. R. & Xie, X. S. Genome-wide detection of single nucleotide and copy-number variations of a single human cell. Science 338, 1622-1626 2012). Single-cell genome sequencing reveals genetic heterogeneity of single cells; applications include analysis of de novo germline mutations or somatic mutations in normal and cancer cells, however as the transcriptome is also representing a part of these mutations, transcriptomic profiling is also used to elucidate mutational landscapes, especially for B-cell based somatic hypermutation phenotypes (King H W, Orban N, Riches J C, et al. Antibody Repertoire and Gene Expression Dynamics of Diverse Human B Cell States During Affinity Maturation. SSRN Electron J. 2020; 2:1-33. doi:10.2139/ssrn.3608389).

The currently used methods are also aimed at capturing the transcriptome, e.g., Smart-seq-WTA with template switching (Ramsköld, D. et al. Full-length mRNA-Seq from single-cell levels of RNA and individual circulating tumor cells. Nat. Biotechnol. 30, 777-782 2012), CEL-seq-WTA with in vitro transcription (Hashimshony, T., Wagner, F., Sher, N. & Yanai, I. CEL-Seq: single-cell RNA-seq by multiplexed linear amplification. Cell Rep. 2, 666-673 2012), Quartz-seq-WTA with poly(A) tagging (Sasagawa, Y. et al. Quartz-Seq: a highly reproducible and sensitive single-cell RNA-Seq reveals non-genetic gene expression heterogeneity. Genome Biol. 14, R31 2013), C1-CAGE-5′-end RNA-seq (Kouno, T. et al. C1 CAGE detects transcription start sites and enhancer activity at single-cell resolution. Nat. Commun. 10, 360 2019), RamDa-seq—full-length RNA-seq (Hayashi, T. et al. Single-cell full-length total RNA sequencing uncovers dynamics of recursive splicing and enhancer RNAs. Nat. Commun. 9, 619 2018), Drop-seq—microdroplet-based (Macosko, E. Z. et al. Highly parallel genome-wide expression profiling of individual cells using nanoliter droplets. Cell 161, 1202-1214 2015), Microwell-seq-microwell-based (Han, X. et al. Mapping the mouse cell atlas by microwell-seq. Cell 172, 1091-1107.e17 2018). Bulk sample RNA-seq provides measurements for only the average transcript expression in a cell population, thus, to precisely describe the transcriptomes of single cells of heterogeneous cell populations, the single-cell RNAseq methods provide a more comprehensive description of the state of cells including the transitions between states. scRNAseq transcriptome analysis has been toward detection of the temporal patterns of the transcriptome to even better define transition in a pseudo-time space (La Manno G, Soldatov R, Zeisel A, et al. RNA velocity of single cells. Nature. 2018; 560(7719): 494-498. doi: 10.1038/s41586-018-0414-6).

The currently used methods are also aimed at capturing other aspects of cellular physiology (or epigenome), including accessible chromatin, methylation and histone modifications, e.g., scBS-seq—DNA methylation (Smallwood, S. A. et al. Single-cell genome-wide bisulfite sequencing for assessing epigenetic heterogeneity. Nat. Methods 11, 817-820 2014), scRRBS—DNA methylation (Guo, H. et al. Single-cell methylome landscapes of mouse embryonic stem cells and early embryos analyzed using reduced representation bisulfite sequencing. Genome Res. 23, 2126-2135 2013), scAba-seq-DNA methylation 5hmC (Mooijman, D., Dey, S. S., Boisset, J.-C., Crosetto, N. & van Oudenaarden, A. Single-cell 5hmC sequencing reveals chromosome-wide cell-to-cell variability and enables lineage reconstruction. Nat. Biotechnol. 34, 852-856 2016), scATAC-seq—Chromatin accessibility (Buenrostro, J. D. et al. Single-cell chromatin accessibility reveals principles of regulatory variation. Nature 523, 486-490 2015), Drop-ChIP—Histone modification ChIP-seq (Rotem, A. et al. Single-cell ChIP-seq reveals cell subpopulations defined by chromatin state. Nat. Biotechnol. 33, 1165-1172 2015), scChIC-seq-Histone modification (Ku, W. L. et al. Single-cell chromatin immunocleavage sequencing (scChIC-seq) to profile histone modification. Nat. Methods 16, 323-325 2019), CUT&Tag—Histone modification (Kaya-Okur, H. S. et al. CUT&Tag for efficient epigenomic profiling of small samples and single cells. Nat. Commun. 10, 1930 2019), scHi-C—Chromatin structure (Nagano, T. et al. Single-cell Hi-C reveals cell-to-cell variability in chromosome structure. Nature 502, 59-64 2013).

The currently used methods are also aimed at capturing the combined workflow to measure the different aspects of the cellular physiology in a combined manner, motivated by lack of reproducibility of the single-cell objects, including the combination of genomic, transcriptomic, open chromatin, methylation or even surface proteins analytical approaches, e.g. G&T-seq—genome and transcriptome: FACS based MDA/PicoPlex (WGA), SMART-seq2 (Macaulay, I. C. et al. G&T-seq: parallel sequencing of single-cell genomes and transcriptomes. Nat. Methods 12, 519-522 2015), DR-seq—DNA and RNA sequencing (Dey, S. S., Kester, L., Spanjaard, B., Bienko, M. & Van Oudenaarden, A. Integrated genome and transcriptome sequencing of the same cell. Nat. Biotechnol. 33, 285-289 2015), scM&T-seq—DNA methylation and transcriptome (Angermueller, C. et al. Parallel single-cell sequencing links transcriptional and epigenetic heterogeneity. Nat. Methods 13, 229-232 2016), scDam&T-seq—chromatin, transcriptome based on FACS (384 well plate) combination of DamID and CEL-seq (Rooijers, K. et al. Simultaneous quantification of protein-DNA contacts and transcriptomes in single cells. Nat. Biotechnol. 37, 766-772 2019), T-ATAC-seq—open chromatin, based on scATAC-seq and TCR-seq (Satpathy, A. T. et al. Transcript-indexed ATAC-seq for precision immune profiling. Nat. Med. 24, 580-590 2018), SNARE-seq-open chromatin and transcriptome (Chen, S., Lake, B. B. & Zhang, K. High-throughput sequencing of the transcriptome and chromatin accessibility in the same cell. Nat. Biotechnol. 37, 1452-1457 2019), scCAT-seq—open chromatin and transcriptome based on FACS (Liu, L. et al. Deconvolution of single-cell multi-omics layers reveals regulatory heterogeneity. Nat. Commun. 10, 470 2019), CITE-seq—surface protein and transcriptome (Stoeckius, M. et al. Simultaneous epitope and transcriptome measurement in single cells. Nat. Methods 14, 865-868 2017), REAP-seq—surface protein and transcriptome (Peterson, V. M. et al. Multiplexed quantification of proteins and transcripts in single cells. Nat. Biotechnol. 35, 936-939 2017).

Simultaneous detection, which are more limited in scale measuring transcripts and proteins in single cells, are also devised, including indexed cell sorting using a combination of RNA sequencing (Paul, F. et al. Transcriptional Heterogeneity and Lineage Commitment in Myeloid Progenitors. CELL 163, 1663-1677 2015, Wilson, N. K. et al. Combined Single-Cell Functional and Gene Expression Analysis Resolves Heterogeneity within Stem Cell Populations. CELL STEM CELL 16, 712-724 2015), or proximity ligation assay (PLA and its variations) in combination with PCR (Stahlberg, A. et al. Quantitative PCR analysis of DNA, RNAs, and proteins in the same single cell. Clinical Chemistry 58, 1682-1691, 2012, Genshaft, A. S. et al. Multiplexed, targeted profiling of single-cell proteomes and transcriptomes in a single reaction. Genome Biol. 17:188, 2016, Albayrak, C. et al. Digital Quantification of Proteins and mRNA in Single Mammalian Cells. Molecular Cell 61, 914-924, 2016, Darmanis, S. et al. Simultaneous Multiplexed Measurement of RNA and Proteins in Single Cells. Cell Reports 14, 380-389, 2016), or mass cytometry (Frei, A. P. et al. Highly multiplexed simultaneous detection of RNAs and proteins in single cells. Nature Methods 13, 269-275, 2016). For example, Albayrak et al, 2016, and Hu et al., 2019 (Hu, Jiumei, et al, “Proximity ligation assays for precise quantification of femtomolar proteins in single cells using self-priming microfluidicd PCR chip”, ANALYTICA CHIM-ICA ACTA, vol. 1076, Jan. 10, 2019) optimize their PLA reaction only regarding the optimal concentration of a PLA probe for detecting a certain target concentration. For the final quantification of the analyzed protein in a sample these approaches also merely consider the data of said simple calibration/titration curves of the analyzed protein and/or PLA probes (nucleic acid-coupled antibodies). Hence, the quantification performed in the prior art only considers the potential bias introduced by different concentrations of target and/or PLA probe during the PLA assay, no further characteristics of the PLA probes are analyzed or considered, such that no absolute quantification is possible in said methods. Further, these assays are limited in scale and/or can only profile a few genes and proteins in parallel and restricted to extracellular proteins without absolute quantification and unable to address interactomics, complexomics of single-cells, while epi-proteomics is severely limited. Conventional techniques such as PLA (proximity ligation assay) and other applicable methods often fail to detect all molecules accurately and exhibit background noise, leading to false detection of non-existent molecules. As a consequence, these techniques do not provide an absolute count of molecules, which is essential for reference-free absolute quantification. The method of invention address these limitations by enabling precise and accurate quantification of molecules without the need for a reference, thereby overcoming the shortcomings of existing techniques.

Drop-seq, CITE-seq and REAP-seq apply a sequencing-based readout for protein levels by conjugating antibodies to poly-A tagged oligonucleotides (antibody-derived tags-ADTs), which are captured concurrently by the cDNA capture. The antibodies are identified by a barcode included in the label. The labeled antibodies bind to their targets in single-cell suspensions and the cells are washed to remove unbound antibodies and processed for scRNA-seq. Typically, the antibody-decorated single cells are encapsulated into nanoliter-sized reaction droplets to carry out the lysis of the single cell, extracting the cellular mRNAs along with antibody labels and converting them by their 3′ polyA sequences to DNA. The transcribed cDNAs and ADTs converted into NGS libraries and sequenced revealing the quantification of cDNAs and ADTs. These assays are limited similarly to the widely applied FACS (fluorescent activated cell sorting) (Bonner W A, Hulett H R, Sweet R G, Herzenberg L A. Fluorescence activated cell sorting. Rev Sci Instrum. 1972; 43(3):404-409. doi:10.1063/1.1685647) as the detection of proteins carried out using FACS protocols including washing steps, low specificity and background, and providing only relative semi-quantification results, which cannot be compared between antibodies categorizing these methods as not absolutely quantitative methods. The application of ADTs is unable to address interactomics, complexomics of single-cells, and detection of intracellular proteins is severely limited. As consequence, these assays are limited to the cell surface proteins as their intracellular application results in high background, loss of even semi-quantification due to poorly controllable and unavoidable washing steps.

Other method for cell-surface-protein analysis methods (Labib, M., & Kelley, S. O. (2020). Single-cell analysis targeting the proteome. Nature Reviews Chemistry, 4(3), 143-158) include magnetic ranking cytometry (MagRC) and flow cytometry (FACS). Methods that can be used for the analysis of generally applicable for detection of proteins include single-cell Western blotting, microfluidic proximity ligation assay (PLA), proximity extension assay (PEA), mass cytometry (cytometry by time of flight; CyTOF) and mass spectrometry and single-cell barcode chips (SCBCs). However, all of these methods have limitation especially providing absolute quantitative detection and high sensitivity, current best sensitivities limited to a few 1000 s of molecules per single-cells, while having high background (C. Albayrak, C. A. Jordi, C. Zechner, J. Lin, C. A. Bichsel, M. Khammash, S. Tay, Digital Quantification of Proteins and mRNA in Single Mammalian Cells, Mol. Cell. 61 (2016) 914-924. https://doi.org/10.1016/j.molcel.2016.02.030.), regardless of the cellular compartments while accessing either proteomic, interactomic, epi-proteomic and complexomic targets.

Thus, more efficient compositions, highly sensitive, accessing intracellular targets revealing proteomic, interactomic, epi-proteomic and complexomic information and having universally absolutely quantitative, comparable results are necessary for quantifying the multitude of single-cellular targets for diagnostic and research applications.

### SUMMARY OF THE INVENTION

In light of the prior art the technical problem underlying the present invention is to provide improved methods for the reliable quantification of target analytes on a single-cell level, such as, for example, proteins, protein-modifications, DNA-modifications, protein-protein-, protein-DNA- or protein-RNA complexes regardless the cellular localization of the targets and in an absolute quantitative manner.

This problem is solved by the features of the independent claims. Preferred embodiments of the present invention are provided by the dependent claims.

The invention therefore relates in one aspect to a method for determining the absolute concentration of at least one target analyte in a sample comprising:
a. providing at least a binding agent of a first type and a binding agent of a second type, each comprising a label that is unique for the type of binding agent, wherein the binding agent of a first type and the binding agent of a second type specifically bind to a first target analyte,
b. determining the binding characteristics of the binding agent of a first type and the binding agent of a second type to the first target analyte,
c. providing a sample comprising cells at a known concentration,
d. bringing the binding agent of a first type and the binding agent of a second type into contact with the sample, wherein the binding agent of a first type and the binding agent of a second type bind to the first target analyte forming an analyte-binding agent complex (couplexes), preferably under suitable conditions,
e. compartmentalizing (single) cells of the sample into a plurality of first partitions, wherein preferably each compartment comprises no more than one cell (or one or no cell),
f. complementing each first partition comprising a single cell with a lysis buffer, preferably a lysis and/or dispersion buffer, thereby lysing the single cell in each of the first partitions,
g. compartmentalizing single analyte-binding agent complexes (couplexes) into a plurality of second partitions,
h. performing a bi-component detection method, thereby determining the number and/or concentration of couplexes and binding agents in the second partition,
i. determining the absolute concentration of the first target analyte per cell in the first partition by taking into account the initial concentration of the cells in the sample in step a., the binding characteristics determined in step b. and the number and/or concentration of couplexes and binding agents determined in step h. in the second partition.

In embodiments one or more of the steps of the afore method may be performed in a different or adapted order. In some embodiments certain steps are performed in parallel. In embodiments, for example, depending on the cellular localization of the target analyte, the step d. of ‘bringing the binding agent of a first type and the binding agent of a second type into contact with the sample’ may be performed before or after the first compartmentalization step e., wherein, preferably single cells, of the sample are compartmentalized (such that a compartment preferably comprises no more than 1 cell, or one or no cell) and after cell lysis and/or addition of a lysis or dispersion buffer within each respective first compartment (step f). In such embodiments especially target agents comprised within the nucleus of a cell and/or other cellular organelles may be detected/bound by binding agents, as the nuclear or other membranes have already been dissolved in the lysis step f. before the incubation with binding agents (in step d.).

Hence, in embodiments the steps of the afore method may be performed for example in the order of step a., step b., step c., step e., step f., step d., step g., step h. and step i.

Therefore, in embodiments the method comprises the (order of) steps of:
a. providing at least a binding agent of a first type and a binding agent of a second type, each comprising a label that is unique for the type of binding agent, wherein the binding agent of a first type and the binding agent of a second type specifically bind to a first target analyte,
b. determining the binding characteristics of the binding agent of a first type and the binding agent of a second type to the first target analyte,
c. providing a sample comprising cells at a known concentration,
e. compartmentalizing the sample into a plurality of first partitions, wherein preferably each compartment comprises no more than one cell,
f. complementing each first partition comprising a sample with a lysis buffer, preferably a lysis and/or dispersion buffer, thereby lysing the single cell in each of the first partitions,
d. bringing the binding agent of a first type and the binding agent of a second type into contact with the sample in the first partition, wherein the binding agent of a first type and the binding agent of a second type bind to the first target analyte forming an analyte-binding agent complex (couplexes), preferably under suitable conditions,
g. compartmentalizing single analyte-binding agent complexes (couplexes) into a plurality of second partitions,
h. performing a bi-component detection method, thereby determining the number and/or concentration of couplexes and binding agents in the second partition,
i. determining the absolute concentration of the first target analyte per cell in the first partition by taking into account the initial concentration of the cells in the sample in step a., the binding characteristics determined in step b. and the number and/or concentration of couplexes and binding agents determined in step i. in the second partition.

In some embodiments the binding reaction d. is performed after a first compartmentalization of sample components, e.g., single cells, and under lysing and/or dispersion buffer conditions, e.g., after and/or during addition of cell lysis and/or dispersion buffer to each first compartment and after and/or during incubation with said buffer.

In embodiments the steps of cell lysis/permeabilization (step f.) and the binding reaction (step d.) are performed in parallel or at the same time. Hence in such embodiments the steps of the afore method may be performed in the order of step a., step b., step c., step e., step f.+step d., step g., step h. and step i. In such embodiments the combined step f.+d. comprises: complementing each first partition comprising a sample with a lysis buffer (preferably a lysis and/or dispersion buffer) and a binding agent of a first type and the binding agent of a second type, thereby lysing the single cell in each of the first partitions and bringing the binding agent of a first type and the binding agent of a second type into contact with the sample in the first partition and, wherein the binding agent of a first type and the binding agent of a second type bind to the first target analyte forming an analyte-binding agent complex (couplexes), preferably under suitable conditions. In some of said embodiments the binding agents are added to the first compartments comprised within a lysis and/or dispersion buffer (in step f./d.), or the binding agents are comprised within said lysis and/or dispersion buffer when being added to the first compartments (in step f./d.).

In some of said embodiments with amended order of the method steps, the compartmentalization/isolation of single cells is preferably performed before the cell lysis, such that only lysates of single cells are analyzed (and not bulk lysates).

Absolute quantification is a critical feature of analytical methods that provide precise measurements of the number of molecules in a sample without relying on external quantitative standards. Achieving absolute quantification is challenging in the study of biological systems due to the large variations in protein properties, the wide range of protein abundance, and the complex cellular environment. Existing absolute quantification methods, mainly based on mass spectrometry, are only partially established and lack approaches that do not require external standards.

The bi-component method preferably used in embodiments herein, constitutes preferably an external quantitative reference-free protein assay. It preferably utilizes a two-step readout method to achieve high signal-to-noise performance without background noise. In a first step, the partitioning of couplexes preferably ensures robust signal generation and mathematically traceable results. The formation of couplexes under stringent buffer conditions avoids matrix effects and guarantees accurate quantification. In a second step the present method employs in embodiments compartmentalized, single-molecular digital PCR for precise quantification of the couplexes. This step is preferably separated from the antibody binding process/step to ensure an absolute signal based on the single molecule sensitivity of digital PCR readings.

The presently employed bi-component detection method offers several advantages over existing assays. It provides a reference-free absolute quantification method that eliminates the need for external standards, making it highly versatile and applicable to diverse protein analytes. The homogeneous, preferably no-wash workflow of the present bi-component method minimizes signal loss and avoids challenges associated with solid phase capture steps. The assay's reliance in embodiments on couplex formation and compartmentalization ensures preferably a strict dependency on binding agent binding, preventing dissociation-related issues and providing robust quantification. In embodiments by detecting and quantifying the couplex and individual binding agent counts, the presently used bi-component method enables extensive control over reaction readings, including determination of binding concentrations, normalization of parallels, and offsetting dPCR clustering errors.

The calculation involves in embodiments the determination of the correct concentrations of the binding reactants in the binding reaction including the binding agents and couplexes, these number might in embodiments be corrected by labeled ratio (LR), label synthesis error (LE), the activity fraction (AF) of binding agents and/or the contamination with free labels (FL), also in embodiments by proper compensation mathematical errors, like clustering of induvial compartments according their positive or negative rating regarding containing different molecular entities. In embodiments the corrected concentrations can be directly used to calculate the absolute concentration of analytes using e.g., the in WO 2020/260277 A1 described method, comprised as a reference herein, that applies chemical balances to determine absolute concentrations.

In the present invention, one aspect of embodiments of the absolute quantification method may be highlighted, such as the saturation condition. This condition preferably enables a direct correspondence between the concentration of the ternary complex (couplex), formed during the assay process, and the target analytes, preferably without the need to determine a binding agents (e.g., antibody's) dissociation constant (Kd). In embodiments by ensuring saturation of the assay components, the concentration of the couplexes can be reliably measured, providing an accurate and straightforward means of quantifying the target analytes. This approach simplifies the absolute quantification process, preferably eliminating the requirement to determine Kd values for the binding agents (e.g., antibodies) used, and enabling a direct and precise quantification of the analytes of interest.

The step of double compartmentalization (the compartmentalization is defined as having a multitude of separated, reproduced, partitions with specific conditions) and using an absolute quantitative method to derive the absolute chemical conditions in the first compartmentalization (first partition) from the second compartmentalization (second partition), represents a new concept over the prior art, as it combines the advantages of two different conditions at the same time:

During the first compartmentalization the binding reaction has preferably a high local target concentration, which could be of two different types:
(1) it is a suspension of a large number of cells, where the compartmentalization is diffusion-limited and the cells are in their own concentration environment achieving a high local concentration of the targets, or
(2) a few cells or single-cells are in a small volume having so a high local target concentration.

In the present concept preferably the first compartmentalization and the second compartmentalization are interconnected:
(3) the absolute chemical correspondence between the first and second compartmentalizations enables the determination of absolute chemical conditions in the first compartmentalization (first partitions) of single-cells from the second compartmentalization (second partitions), without determination of absolute chemical conditions in the first compartmentalization absolute target concentrations cannot be derived,
(4) provides high precision in the determination of absolute quantities of the in single cells, needed for single cells, using the determination of binding characteristics of binding agents. With respect to the difference and improvement over standard binding reactions in bulk solutions, the present single cell binding reaction is compartmentalized and therefore maintains the separability of cells, while in reactions with batch-lysed cells the results are non-separable for each cell while enables the absolute chemical correspondence between compartmentalizations.

For example, in preferred embodiments the step of the second compartmentalization is used to derive/reveal the unknown absolute chemistries (the absolute concentration of binding agents and couplexes) that are present in the first compartmentalization (in each of the first partitions, each comprising the contents of a single cell and potentially binding agents).

In embodiments the unit of detection is defined as the complex of the target and two bound binding agents and called couplex. If more than two binding agents are bound, all unique combinatorial combinations of binding agent pairs are considered as distinct couplexes.

In preferred embodiments the second compartmentalization provides exact numbers of molecules such that the absolute amount/concentration of the target analyte from determined absolute chemistries and additionally by taking into account the volume of the first compartmentalization (first partition) and/or the initial concentration of cells in the initial solution provided for first compartmentalization, and/or the initial concentration of the binding agents in the initial solution provided for first compartmentalization and, most importantly, the previously determined (apparent) binding properties of the binding agents to determine the absolute amounts/concentrations.

Preferably the bi-component detection method determines the number and/or absolute concentration of the binding agents in addition to the number and/or absolute concentration of couplexes in the second partition in step h. Accordingly, the bi-component detection method is applied also to determine the number and/or absolute concentration of binding agents in the initial first partition (which was split into a plurality of second partitions comprising its contents).

In summary, the invention surprisingly facilitates the reliable and exact quantitative assessment of the absolute concentration (amount) if different kinds of binding agent-targets (target analytes) in single-cells.

The invention achieves it's surprising effect preferably by two major improvements over the art, (i) using target material of unknown quantity to determine the binding characteristics of binding agents, such as antibodies, considering preferably the apparent dissociation constant of the binding agents, e.g., antibodies considering the labeled ratio of the binding agents, and/or the label synthesis error rendering the label undetectable, and/or the active fraction of the binding agent, which has the capability of binding and the contamination with free labels.

Subsequently, based on these measures the apparent dissociation constant of the binding agents, e.g., antibodies, is preferably determined. This is followed by a (ii) double compartmentalization of single-cell-components enabling the absolute quantitative correspondence between conditions of first compartmentalization of single-cells during the binding incubation of the binding agents, and the second compartmentalization of single-cell components (e.g., complexes formed by binding agents and target molecules), which enables, through consideration of the previously determined binding characteristic of the binding agents, the determination of extremely low (down to 10 target analytes per single-cell) and absolute amounts of the target analytes in each single cell.

In embodiments the binding characteristics said active fraction and said contamination with free label are inherently compensated by the second compartmentalization method, which represents a surprising finding and feature of the present method.

Hence, in preferred embodiments of the method according to the invention determining the binding characteristics in step b. comprises determining for each binding agent of a first and a of a second type: the dissociation constant of from the (respective) target analyte, the labeled ratio and the label synthesis error.

In other words, in preferred embodiments in step b. determining the binding characteristics, comprises determining the (apparent) dissociation constant for each binding agent of a first and a of a second type by taking into account the labeled ratio and the label synthesis error of the binding agent(s), wherein the active fraction and the free label are self-compensating.

In preferred embodiments, the present method determines, using labeled ratio and the label synthesis error of binding agent(s), the apparent dissociation constant of said binding agent(s), thereby providing an advantageous self-compensation inherently for the active fraction of the binding agent(s) and the contamination with free labels to provide the dissociation constant of antibodies for the unbiased determination of absolute quantities in single-cells.

In embodiments determining the binding characteristics in step b. comprises determining for each binding agent of a first and a of a second type: the dissociation constant of from the target analyte, the labeled ratio, the labeling error ratio, the activity ratio and/or the optimal concentration (number of molecules per unit volume) required for binding to the target analyte.

In embodiments determining the binding characteristics in step b. comprises determining for each binding agent of a first and a of a second type the dissociation constant of from the target analyte. In embodiments determining the binding characteristics in step b. comprises determining for each binding agent of a first and a of a second type the labeled ratio.

In embodiments determining the binding characteristics in step b. comprises determining for each binding agent of a first and a of a second type the labeling error ratio. In embodiments determining the binding characteristics in step b. comprises determining for each binding agent of a first and a of a second type the labeled ratio and/or the labeling error (label error; label synthesis error (LE)) ratio.

In embodiments determining the binding characteristics in step b. comprises determining for each binding agent of a first and a of a second type the contamination with free labels (FL).

In embodiments determining the binding characteristics in step b. comprises determining for each binding agent of a first and a of a second type the labeling error ratio.

In embodiments determining the binding characteristics in step b. comprises determining for each binding agent of a first and a of a second type the labeled ratio, the labeling error (label error; label synthesis error (LE)) ratio and/or the contamination with free labels (FL).

In embodiments the determination efficiency and correctness of the labelling of a binding agent, namely the ‘labeled ratio’ and ‘labelling error’ ratios, but in embodiments also the contamination of a binding agent solution with free labels (after labeling of the binding agents), facilitates a correction of label-related errors from the final results, such as the reading of the labels sequences. For example, if a binding agent does not comprise a label (labeled ratio) or the label comprises a sequence error and/or the binding agent comprises a wrong label, said binding agent cannot be detected and/or identified during later data analysis. A contamination of a sample with free labels can lead to a false-positive detection of couplexes. Accordingly, couplexes comprising a wrongly-labelled or un-labelled binding agent cannot be identified in later data analysis. Hence, a prior determination of the efficiency and correctness of the labelling of binding agents facilitates a later correction for said errors, such that the precision of the determination/calculation of the concentration of a target analyte in a sample can be further improved.

In embodiments determining the binding characteristics in step b. comprises determining for each binding agent of a first and a of a second type the activity ratio.

In embodiments determining the binding characteristics in step b. comprises determining for each binding agent of a first and a of a second type the optimal concentration (number of molecules per unit volume) required for binding to the target analyte.

In embodiments determining the binding characteristics in step b. comprises determining for each binding agent of a first and a of a second type: the dissociation constant of from the target analyte, the labeled ratio, the labeling error ratio, and/or the activity ratio.

In embodiments determining the binding characteristics in step b. comprises determining for each binding agent of a first and a of a second type the labeled ratio, the labeling error ratio, and/or the activity ratio.

In embodiments the binding characteristics of binding agents, preferably comprise their (re) activity, specificity, concentration, labeled ratio (LR), their label synthesis error (LE), the activity fraction (AF) of binding agents and/or the contamination with free labels (FL),

The determination of so-termed ‘binding characteristics’ for binding agents herein thus represents an improved approach for the optimization of absolute quantification of a target analyte concentration in a sample. By determining and mitigating the “binding characteristics” associated with binding agents, the present method surprisingly enables the identification of specific conditions where the effect of some of these characteristics preferably becomes negligible on absolute quantification. Specifically, in embodiments the invention focuses on establishing saturation conditions, characterized by a high concentration of binding agents that render the effect of their reactivity negligible on the determination of absolute quantification. Application of the quantification method under these optimized saturation conditions ensures accurate and reliable quantification of the target analyte concentration.

The determination of so termed ‘binding characteristics’ for binding agents herein thus represents an improved approach for the optimization of absolute quantification of a target analyte concentration in a sample. This is, as additional confounders of the calculation of the absolute quantification of a target analyte, such as un-labelled or falsely labelled binding agents and/or the dissociation constant between a binding agent and its target analyte, can be identified and can be taken into account during the final calculation of the target concentration.

In preferred embodiments step h. of performing a bi-component detection method, comprises thereby determining the number and/or absolute concentration of couplexes and binding agents in the second compartment.

The binding characteristics of a binding agent may also include reactivity, specificity and/or the optimal concentration (number of molecules per unit volume) required for binding to the target analyte. Reactivity is described by the dissociation constant of the binding agent, specificity described by identifying binding targets specifically and concentration is described by number of molecules per unit volume. WO 2020/260277 A1 describes a method, as a reference herein, that applies chemical balances to determine dissociation constants of binding agents using the described binding characteristics.

The present invention significantly improves the method of WO 2020/260277 A1 and extends it to be used for highly precise and sensitive single-cell measurements including additional characteristics such as the labeled ratio of antibodies, the label synthesis error rendering the label undetectable, and/or the active fraction of the antibody, which has the capability of binding and the contamination with free labels. The present method subsequently facilitates determining, based on these measures, the apparent dissociation constant of binding agents, such as antibodies, providing in conjunction with a double-compartmentalization of single-cell-components as described precise, high sensitivity detection of target analytes, down to about 10 target analytes per single-cell.

In this context the labeled ratio (LR) of binding agents describes the ratio of the label bearing and label not bearing fractions of binding agents. The label synthesis error (LE) describes the fraction of undetectable labels, e.g., due to sequence errors of labels. It can be relevant for the precision of embodiments of the present method to determine the label synthesis error or short “label error”, as the binding of a binding agent to a target analyte could not be detected, if it comprises an undetectable label. The active fraction (AF) of binding agents describes the ratio of reactive and non-reactive fractions of binding agents. The contamination of binding agents with free labels (FL) describes the fraction of labels not bound to a binding agent during the binding agent preparation and bears the danger of false positive results during the measurements of target analytes with the present method.

The present invention describes a method to use the binding characteristics of binding agents, preferably their reactivity, specificity, concentration, labeled ratio (LR), their label synthesis error (LE), the activity fraction (AF) of binding agents and/or the contamination with free labels (FL), to determine a novel corrective, e.g., apparent dissociation constant of binding agents, to be used for determining absolute quantities of target analytes. Since the determined binding characteristics expand on the molecular binding characteristics by taking into account practical limitations (such as activity fraction (AF) of binding agents etc.) the binding characteristics may also be referred to as extended binding characteristics. In one preferred embodiment the system bearing disperse, discontinuous concentration of target analytes e.g., suspension of single-cells.

In preferred embodiments the present invention applies the determination of the (extended) characteristics of binding agents to enable the determination of extremely low and absolute amounts of targets of single-cells, preferably down to 100 s or preferably 10 s of targets per single-cell.

In embodiments the bi-component detection method in step h. comprises the amplification of nucleic acids or a nucleic acid amplification step.

In embodiments the binding agent of a first type and the binding agent of a second type, each comprise a nucleic acid label, which is preferably unique for the type of binding agent, and the bi-component detection method in step h. comprises amplifying, preferably inside each of the second partitions, the nucleic acid sequence label of binding agent.

In embodiments the amplification comprises the linking of the nucleic acid sequence of the nucleic acid labels of two or more binding agents forming a couplex, preferably wherein the linking may be the synthesis of a nucleic acid sequence complementary to the single-stranded nucleic acid labels of the binding agents. In such embodiments not the labels themselves are linked, but their nucleic acid sequence in a nucleic acid amplification step. Therefore in embodiments the linking (inside each second partition) of the nucleic acid sequence label of the binding agent of a first type to the nucleic acid sequence label of the binding agent of a second type can be understood to be interchangeable/synonymic with the linking/combination of the nucleic acid sequences (sequence of nucleotides) of said labels. The result of said linking and/or amplification step is preferably a nucleic acid sequence comprising each nucleic acid sequence of the labels of the at least two binding agents forming a couplex, such that the identity of the binding agents and hence also the identity of target analytes in a complex can be determined.

In embodiments the bi-component detection method in step h. comprises combining (pooling) the plurality of second partitions.

In embodiments of the present method the bi-component detection method in step h. comprises performing nucleic acid sequence analysis.

In embodiments of the method according to the invention the bi-component detection method in step h. comprises performing nucleic acid sequence analysis, such as e.g., nucleic acid sequencing or PCR analysis, thereby determining the number and/or identity of nucleic acid sequences present in the sample. In embodiments nucleic acid sequence analysis, such as e.g., nucleic acid sequencing or PCR analysis, is performed to determine the number and/or identity of nucleic acid sequence labels (of binding agents). In embodiments nucleic acid sequence analysis is performed and thereby the number and/or identity of nucleic acid sequence labels (of binding agents) is determined, subsequently from the number and/or identity of, preferably unique, nucleic acid sequence labels the number and/or concentration of couplexes is calculated.

In exemplary embodiments, where two different binding agents, each comprising a unique nucleic acid label that also is characteristic for the respective type of binding agent, bind to a target analyte (form a couplex) and are compartmentalized (separated) into a second compartment, the two unique sequences of said nucleic acid labels may be joined/combined by a nucleic acid amplification step. The obtained sequence comprises the joined/combined nucleic acid sequences of the two labels, such that by nucleic acid sequence analysis the identity of the two different binding agents (based on the joined label sequences) and hence of the target analytes forming a couplex can be determined. As the labels preferably comprise a unique sequence, duplicates that arise e.g., during amplification and/or sequence analysis can be discarded/ignored during the final analysis. Further, in embodiments the identity and/or number of binding agents forming couplexes with one or more target analytes can then be normalized by the initial concentration of the binding agents in the biding reaction (step d.) and/or one or more binding characteristics of the binding agents, e.g., the labeled ratio, the label synthesis error, the dissociation constant from the target analyte etc., and is included in the calculation of the normalization process as described in detail herein.

In embodiments of the present method the absolute concentration of the first target analyte per cell in the first partition is calculated from the initial concentration of the cells in the sample (as adjusted/determined in step a.), the binding characteristics determined in step b. and the number and/or concentration of couplexes and binding agents in step h. in the second partition and thereby the number and/or concentration of couplexes.

In embodiments of the method according to the invention the binding agent of a first type and the binding agent of a second type, each comprise a nucleic acid label that is unique for the type of binding agent, and the bi-component detection method in step h. comprises the steps of:
amplifying inside each of the second partitions the nucleic acid sequence labels, and
combining the plurality of second partitions and performing nucleic acid sequence analysis in order to determine the number and identity of nucleic acid sequence labels and thereby the number and/or concentration of couplexes.

In embodiments of the present method the bi-component detection method in step h. comprises performing a linking step between the label of the binding agent of a first type and the binding agent of a second type in each second partition, this linking step provides the co-compartmentalization information of the bi-component method.

In embodiments of the present method the linking step in step h. comprises linking inside each second partition the nucleic acid sequence label of the binding agent of a first type to the nucleic acid sequence label of the binding agent of a second type if both form a couplex with the first target analyte.

In some embodiments the linking step comprises the performance of nucleic acid amplification (e.g., PCR or digital PCR or linkage PCR) of the nucleic acid sequence labels. In other embodiments the linking step is a classical proximity ligation reaction involving rolling circle DNA synthesis or rolling circle amplification. In other embodiments the ligation is achieved using a ligase enzyme. In subsequent nucleic acid sequencing analysis of the labels, the presence of couplexes in a partition can be evidenced by sequences comprising both the label sequence of a binding agent of a first type and the label sequence of a binding agent of a second type, both binding agents specific for the target analyte and said sequences are associated with co-compartmentalization information.

In any case, the linking step is dependent on the kind of label used. In some embodiments wherein the detection is achieved using fluorescent probes or labels, a linking step between nucleotide labels might not be essentially required, as the presence of couplexes can be detected by the co-localization inside one partition or droplet of two labels with different fluorescent markers or dyes that are used to detect the two different types of binding agents in a couplex, comprising two types of binding agents bound to a target.

In some embodiments, protein or peptide-based labels might be crosslinked according to methods known in the art.

In embodiments of the method according to the present invention during the first compartmentation the cells in the sample are permeabilized (preferably to retrieve an antigen therefrom, in other words the cells are ‘antigen-retrieved’) for the binding agent of a first type and the binding agent of a second type before bringing the binding agent of a first type and the binding agent of a second type into contact with the sample, such that one or more of the binding agent of a first type and the binding agent of a second type enters into one or more of the cells after having been brought into contact with the sample in step d.

In embodiments where the target analyte is located at least partially on the surface or membrane or cell wall of a cell, a permeabilization of the cells may not essentially be required to bring the binding agents into contact with the target analyte. In embodiments, however, where the target analyte is located inside a cell and the binding agents would not be able to interact with the target analyte, as they cannot enter the cells, a permeabilization of the cells inside a sample and/or suspected to comprise the target analyte may be required. Such permeabilization may also comprise the encapsulation of the binding agents into molecular shuttles, such as liposomes, to enable their entrance into a cell. In other embodiments the permeabilization may be achieved using other methods known in the art, such as chemicals (e.g., formaldehyde, triton, tween, NP-40, leucoperm, digitonin, saponin, acetone, methanol etc.), electric current or (ultra) sonification. The person skilled in the art is aware of adequate means for cell permeabilization, dependent on the binding agents and/or cells used.

Hence, in one embodiment the first compartmentalization preferably provides chemical conditions facilitating the detection of targets on the cell surface, intracellularly and/or nuclearly.

In embodiments of the method according to the present invention the binding agent of a first type and the binding agent of a second type, each comprise a nucleic acid label that is unique for the type of binding agent and wherein the plurality of first partitions in step f. are optionally further complemented with PCR-reagents and nucleic acid amplification primers comprising optionally a barcode, which is unique to each first partition and the single cell therein, and optionally RT-PCR reagents, and wherein an additional step of nucleic acid amplification is performed after cell lysis within each second partition detecting additional nucleic acid targets especially specific genomic loci or transcripts or specific couplexes. In embodiments the nucleic acid labels of binding agents present in a couplex may be linked and/or barcoded and/or amplified during such amplification.

After complementing first partition or droplet with lysis buffer optionally further complemented with first strand cDNA synthesis reagents and nucleic acid amplification primers comprising optionally a barcode, which is unique to each first partition and the single cell therein, and wherein an additional step of first strand cDNA synthesis is performed after cell lysis within each first compartments.

In preferred embodiments during said amplification a barcode or UMI unique for each first partition/droplet (or unique for each encapsulated single-cell) is added to each amplified nucleic acid sequence, wherein the unique barcode or UMI is preferably comprised by the sequence of the amplification primers. According to said barcodes or UMIs a single cell-specific analysis of couplexes can be performed during nucleic acid sequencing analysis.

In embodiments of the method according to the present invention the plurality of first partitions in step f and/or preferably step e. are further complemented by a protein-cross-linking agent and/or proteinase inhibitors and/or nuclease inhibitors.

In embodiments of the method according to the present invention the binding agent of a first type and the binding agent of a second type, each comprise a nucleic acid label that is unique for the type of binding agent and wherein each nucleic acid sequence label comprises an identifier sequence (barcode) and a unique molecular identifier (UMI) identifying reactivity of the binding agent and its molecular identity.

The determination of the exact/absolute molecular concentration of a target analyte by the present method can in embodiments be even more improved if the labels of the binding agents are not only specific for the type of binding agent but also unique for each individual binding agent molecule. The use of unique labels for individual binding agent molecules facilitates the determination of the total number of individual couplexes by subsequent nucleic acid sequence analysis when said unique labels are linked to another unique label of the second binding agent within a couplex.

In embodiments of the method according to the present invention the binding agent of a first type and the binding agent of a second type, each comprise a nucleic acid label that is unique for the type of binding agent and wherein the amplification in step h. involves the performance of PCR and/or droplet PCR and/or digital PCR and/or real-time PCR and/or RT-PCR.

In embodiments of the present method more than one target analyte and corresponding first and second type binding agents are provided, and, finally, their absolute concentration is determined according to the method of the invention.

In embodiments of the method according to the present invention the at least one target analyte comprises at least a first and a second target analyte, and wherein in step a. for each of the at least first and second target analytes at least a specific binding agent of a first type and a specific binding agent of a second type are provided.

In embodiments step d. bringing the binding agent of a first type and the binding agent of a second type into contact with the sample, wherein the binding agent of a first type and the binding agent of a second type bind to the first target analyte forming an analyte-binding agent complex (couplexes), is performed under suitable conditions. The phrase ‘under suitable conditions’ refers to any condition described herein that is suitable to enable bringing the binding agent of a first type and the binding agent of a second type into contact with the sample, wherein the binding agent of a first type and the binding agent of a second type bind to the first target analyte forming an analyte-binding agent complex (couplexes). In embodiments suitable conditions may be conditions which comprise or enable a “permeabilization” of cells. In embodiments suitable conditions may comprise a specific “antigen retrieval” condition, such that one or more antigen(s) may be retrieved from the cells. In embodiments the condition of step d. is intended to enable the contact between a binding agent of a first type and the binding agent of a second type to the first target analyte forming an analyte-binding agent complex. Preferably, said condition does not necessarily lyse the cell (release its entire contents), but may in embodiments only permeabilize the cells and/or enable the contact between contents of the cells and binding agent(s), e.g., by enabling the binding agent(s) to enter said cell to interact with its contents (analytes)—if present. In other embodiments the permeabilization of cells prior to binding agent addition may be conceived as a lysis step releasing the contents of the cell into the respective first compartment and/or or enabling the contact between contents of the cells and binding agent(s).

In embodiments a lysis and/or dispersion buffer is used to, or facilitates the full dispersion of cellular material before a subsequent compartmentalization step. In other words, in embodiments the condition of step f. is intended to enable lysing a single cell in each of a first partition to release (disperse) the entire contents of said cell into said first partition. In embodiments at least two different lysis (or lysis-like) conditions and/or buffers are applied during the present method e.g., in steps d. and f. respectively.

The present method also facilitates the detection of more than one target analyte at the same time. In the case of multiple target analytes to be analyzed, for each target analyte two specific binding agents, one of a first and one of a second type have to be chosen. The differentiation between the different pairs of binding agents for the different target analytes can be achieved using, for example, in some embodiments different fluorescent labels, primers or probes during a final PCR, qPCR, real-time PCR or dPCR readout. In one example a probe that specifically binds to the linked sequence of the two binding agent labels for a first target analyte are tagged with one fluorescent color, while a probe specific for the linked binding agent labels for a second target analyte are tagged/marked with a different fluorescent color. In other embodiments where the labels of binding agents within couplexes are not linked, each pair of binding agents in a couplex is detected by a specific combination of fluorescent colors that can be distinguished during final PCR readout.

In embodiments of the method according to the present invention the number of different target analytes is less than 5 and wherein, if a nucleic acid sequence analysis is performed in step h., said analysis comprises digital PCR and/or real-time PCR.

In embodiments wherein the number of target analytes to be analyzed exceeds the number of available fluorescent labels or their combinations nucleic acid sequencing, e.g., NGS, may be used to specifically analyze the abundance of couplexes of said target analytes.

In embodiments of the method according to the present invention the number of different target analytes is more than or equal to 5 and wherein, if a nucleic acid sequence analysis is performed in step h., said analysis comprises Next Generation Sequencing (NGS).

In some embodiments wherein a nucleic acid sequence analysis is performed in step h., which comprises Next Generation Sequencing (NGS), the sequencing data is processed using preferably a custom pipeline. In embodiments said customized pipeline comprises deconvolution of unique sequences comprised within the nucleic acid labels, e.g., UMIs. In embodiments following the description of such deconvolutions the sequences initially comprised within a first or second partition, e.g., droplets, may be deconvoluted as described in WO 2020/260277 A1. Briefly, in embodiments deconvolution of partitions is achieved on the basis of unique pairing between UMIs per partition during the linkage reaction (e.g., PCR) step, such pairings constitute partition-internal pairs of UMIs on a random basis. A unique virtual network can be constructed from the UMI-pairs (of two joined binding agent labels) defining unequivocally the partition (droplet) of linkage reaction (preferably the partition comprising only one couplex), enabling the reconstruction of the droplet (partition) content referring the type of the binding agents present n said couplex/droplet. Based on the content of the droplet the distribution of the binding agents can be determined, so droplets (partitions) with one type of binding agent and droplets with both binding agent present can be distinguished and counted respectively. The overall number of droplets used for sequence analysis (e.g., pooled/combined before sequencing) is derived by direct counting, or using e.g., tracer concepts. The droplet information gained can be evaluated using methods described for dPCR readouts.

Advantageously, the present method facilitates the absolute quantification of target analyte concentration as well as a parallel determination of the concentration of multiple target analytes in a robust and efficient manner. In one embodiment, the present method comprises employing an absolute molecular count based analytical method. Such an absolute molecular count based analytical method preferably refers to applying digital detection methods, such as a digital PCR or NGS analysis.

In embodiments, the present method comprises employing a digital PCR assay. In one embodiment, the present method comprises using target analyte-specific binding agents associated with unique amplifiable nucleic acid labels and employs a compartmentalized assay, wherein a nucleic acid amplification is performed for each partition using preferably fluorescently labelled amplification products or fluorescently labelled probes binding to amplification products of interest.

In some embodiments, the nucleic acid amplification is a PCR and the fluorescently labelled amplification products are fluorescently labelled PCR products. In some embodiments, the nucleic acid amplification is a PCR and the amplification products of interest (labels of binding agents forming a couplex) are detected by fluorescently labelled probes, such as hydrolysis or Taqman probes.

The analyte-specific binding agents may preferably be labelled by unique PCR amplifiable DNA labels, wherein is the two analyte-specific binding agents, e.g., two antibodies, are preferably labeled with a single stranded DNA that is unique for the binding agent of for the type of binding agent (e.g., first type of antibody). In embodiments first the binding characteristic of the labelled binding agents (e.g., antibodies) are determined, followed by their addition to the sample or to the one or more dilutions in order to allow for couplex-formation. Subsequently the reaction is preferably highly diluted, e.g., by a dilution factor of more than 20 000, preferably more than 50 000, 100 000 to achieve single-couplex separation upon a first compartmentalization into first partitions, e.g., by emulsification into droplets using methods known in the art.

In embodiments of the method according to the present invention the binding agent is selected from the group comprising antibodies, engineered protein scaffolds and aptamers.

In embodiments of the method according to the present invention the target analyte is selected from the group comprising peptides, proteins, chemically modified peptides or proteins, nucleic acids, chemically modified nucleic acids and any complexes thereof.

In embodiments not only proteins or peptides comprising specific amino acid sequence motifs or epitopes for antibodies can be analyzed, but also post translational modifications in proteins or peptides, as for example, one type of binding agent may recognize a chemical or post-translational modification and the second type of binding agent may recognize a specific epitope or domain of a protein. The method may also be used to analyze the absolute amount of protein complexes within single-cells, wherein each type of binding agent recognizes one of the proteins forming a complex. But also, nucleic acids or nucleic acid-protein complexes may be recognized by, for example, antibodies specific for said complex members. For, example, one type of binding agent may recognize a protein in a DNA-protein complex, while the other type of binding agent recognizes, e.g., a methylated DNA motif (which might be in a functional context of the interaction). Accordingly, the term ‘any complexes thereof’ may refer to any complex comprising proteins, peptides and/or nucleic acids or any combination thereof, as the only requirement for the present method is that a specific target analyte may be recognized specifically by a pair of two labelled binding agents to form a couplex.

In embodiments of the method according to the present invention the sample is selected from the group comprising a tissue sample, a biopsy, a liquid biopsy, a blood sample, a plasma sample, a urine sample, a liquor sample, an environmental sample, a cell culture-derived sample and a sample derived from a microbiological culture.

In embodiments of the method according to the present invention the sample is provided in step c. after being processed by a method selected from the group comprising fluorescence activated cell sorting (FACS), magnetic-activated cell sorting (MACS), laser capture microdissection (LCM), manual cell picking/micromanipulation, microfluidic separation and on-demand optically intercepted and controlled single-cell printing.

In embodiments the invention relates to a method for determining the absolute concentration of at least one target analyte in a sample comprising:
a. providing at least a binding agent of a first type and a binding agent of a second type, each comprising a nucleic acid sequence label that is unique for the type of binding agent, wherein the binding agent of a first type and the binding agent of a second type specifically bind to a first target analyte, and wherein optionally said nucleic acid sequence label comprises an identifier sequence (barcode) and/or, a unique molecular identifier (UMI),
b. determining the binding characteristics of the binding agent of a first type and of the binding agent of a second type to the first target analyte,
c. providing a sample comprising cells at a known concentration, optionally wherein the concentration comprises or relates to the number of single cells per volume unit and/or the concentration is known/given/determined at a single cell-resolution,
d. optionally permeabilizing the cells in the sample for the binding agent of a first type and the binding agent of a second type,
e. bringing the binding agent of a first type and the binding agent of a second type into contact with the sample,
   wherein optionally one or more of the binding agent of a first type and the binding agent of a second type enters a permeabilized cell, and
   wherein the binding agent of a first type and the binding agent of a second type bind to the first target analyte forming an analyte-binding agent complex (couplex),
f. compartmentalizing single cells of the sample into a plurality of first partitions,
g. complementing each partition comprising a single cell with a lysis buffer and lysing the single cell in each partition,
h. compartmentalizing single analyte-binding agent complexes (couplexes) into a further plurality of second partitions,
i. optionally linking inside each partition the nucleic acid sequence label of the binding agent of a first type to the nucleic acid sequence label of the binding agent of a second type, if both form a couplex with the first target analyte,
j. amplifying inside each partition the linked nucleic acid sequence labels, optionally thereby determining the number of couplexes by detecting partitions comprising both the label of a binding agent of a first type and the label of the binding agent of a second type,
k. combining the plurality of partitions,
l. performing nucleic acid sequence analysis, thereby determining the number and identity of nucleic acid sequence labels linked in step i,
m. determining the absolute concentration of the first target analyte per cell by taking into account the initial concentration of the cells in the sample in step a., the binding characteristics determined in step b. and the number of linked nucleic acid sequence labels determined in step l.

Preferably during step g. the incubation between contents of a cell and the binding agents is still possible/still occurs, such that binding agents may also come into contact (and form couplexes) with target agents comprised within the cell, e.g., within organelles such as e.g., the nucleus, that were before cell lysis not (or less) permeable and/or accessible to binding-agents.

In embodiments the sample comprises cells at a known concentration and wherein the concentration comprises or relates to the number of single cells per volume unit and/or the concentration is known/given/determined at a single cell-resolution, it is particularly beneficial that the present method can also be performed using a single cells, rather instead of a large number of cells or a suspension of a plurality of cells.

In embodiments of the method according to the present invention the binding agent of a first type and the binding agent of a second type, each comprise a nucleic acid label that is unique for the type of binding agent and wherein the plurality of first partitions in step f./g. of complementing each partition comprising a single cell with a lysis buffer and lysing the single cell in each partition, are optionally further complemented with PCR-reagents and nucleic acid amplification primers comprising optionally a barcode, which is unique to each first partition and the single cell therein, and optionally RT-PCR reagents, and wherein an additional step of nucleic acid amplification is performed after cell lysis within each first partition to enable the detection of additional nucleic acid targets, especially specific genomic loci or transcripts or specific couplexes. In embodiments the nucleic acid labels of binding agents present in a couplex may be linked and/or barcoded and/or amplified during such amplification. In some embodiments the sample solution is complemented with PCR and/or reverse transcription and/or RT-PCR reagents before the step of compartmentalizing single-cells into first compartments.

After complementing a first partition with lysis buffer, it is optionally further complemented with first strand cDNA synthesis reagents and nucleic acid amplification primers comprising optionally a barcode, which is unique to each first partition and the single cell therein, and wherein an additional step of first strand cDNA synthesis is performed after cell lysis within each first partition.

In preferred embodiments during said amplification a barcode or UMI unique for each first partition/droplet (or unique for each encapsulated single-cell) is added to each amplified nucleic acid sequence, wherein the unique barcode or UMI is preferably comprised by the sequence of the amplification primers. According to said barcodes or UMIs a single cell-specific analysis of couplexes can be performed during nucleic acid sequencing analysis.

In embodiments step l. of performing nucleic acid sequence analysis comprises in addition determining from the sequence information the RNA composition of said single-cells. Preferably in said embodiment the amplification reaction in step g./j. of amplifying inside each partition the linked nucleic acid sequence labels, further comprises the reverse transcription of RNA into cDNA in a first partition. In other words, in some embodiments of the present invention, the RNA present in each cell can be reverse transcribed during the (initial) amplification step of nucleic acid labels in first partitions. Therefore, the lysis buffer preferably further comprises reverse transcription reagents, or the first partitions or the initial sample solution are complemented with reverse transcription reagents, wherein the generated cDNA may preferably also be labelled during said reverse transcription step with a barcode and/or UMI, enabling the assignment of obtained RNA sequences to first partitions and thereby to single cells. In embodiments the identity/sequence of RNA present in each single-cell can preferably by obtained by determining the nucleic acid sequence of its corresponding cDNA during the final nucleic acid sequence analysis.

Accordingly, in embodiments of the method according to the invention, the sample comprises RNA and wherein the plurality of first partitions in step f./g. are further complemented with reverse transcription reagents, and RNA is converted into cDNA, concurrently during any one or all of steps of e./d. bringing the binding agents of a first and second type into contact with the sample to g. lysis of cells, and wherein, if nucleic acid sequence analysis is performed in step h./l., said analysis comprises Next Generation Sequencing (NGS) or qPCR analysis of the cDNA generated in steps e./d.-g.

In embodiments this present method is advantageous in the determination of absolute amount of target analytes using the method of invention forming physically separated first compartments. In embodiments determining the absolute amount of the target analyte, the first compartmentalization is carried out into first partitions, wherein the compartmentalization is physical, the binding reaction between the cells and the binding agents is preferably carried out at high target analyte-concentration exerted by a low volume encompassing each single-cell. Preferably, the cells are lysed during the first compartmentalization. In embodiments each first compartment is compartmentalized a second time, and a suitable sensitive method is used to count the single target analyte molecules, e.g., nucleic acid sequence analysis. Preferably, the concentration of the target in the first compartment (preferably comprising a known volume and one cell) is determined using the binding characteristics of binding agents, and finally results in the determination of the absolute amount/number of the target analyte per single-cell, as described in the present Examples. In embodiments the method comprises providing a single-cell sample, incubation the sample with two target specific binding agents, wherein the target and at least two binding agents form couplexes, performing compartmentalization of said single-cell sample into a plurality of first partitions (each preferably comprising one or no cell) and complementing each first partition with a cell lysis buffer. During the ‘compartmentalized bi-component method’ (in second partitions) the compositions of the couplexes comprised in the first partitions are determined.

In embodiments the binding agents (e.g., antibodies) are conjugated with DNA amplifiable labels. The achieved labeling efficiencies of the binding agents may in embodiments be determined and may be confirmed in embodiments e.g., by SDS-PAGE electrophoresis. In embodiments free (unbound) labels and the labeling error may be determined (e.g., see Example 5). in embodiments the binding agents (e.g., antibodies) may have preferably no overlapping epitopes and the conjugated binding agents have two different labels. In embodiments the dissociation constants of binding agents may be determined, e.g., as described herein or according to Example 6. In embodiments cells are encapsulated using droplet generation techniques (e.g., microfluidics platforms) with labeled binding agents in first partitions. The resulting emulsion may in embodiments be collected and incubated. In embodiments droplet dispensing may be performed at adjusted droplet concentrations e.g., delivering single first compartments into microplate wells. In embodiments dPCR reagents are added to said compartments and a single droplet may preferably be dispensed into the wells and after mixing loaded onto another well plate. Preferably also a negative control containing only labeled binding agents is also analyzed (binding control). In embodiments dPCR may be performed. In embodiments the count of couplexes and the count of binding agents may be obtained as described herein (e.g. as in Example 6). In embodiments the count (copies) of antibodies expected on average (antibody label ratio and label error corrected) per reactions and the detected couplexes (antibody label ratio and label error corrected) may be determined. In embodiments based on the concentration of binding agents and the predetermined binding characteristics including the Kd of binding agents (e.g., apparent kd determined see Example 6) a calibration curve can be constructed. In embodiments the calculated concentration of target may be determined (on average) per cell.

In embodiments the step of determining the binding characteristics comprises determining for each binding agent of a first and a of a second type:
the dissociation constant of from the target analyte, and/or
the specificity of the binding to the target analyte, and/or
the labeled ratio, and/or
the label synthesis error, and/or
the active fraction, and/or
the contamination with free labels, and/or
the optimal concentration (number of molecules per unit volume) required for binding to the target analyte.

As described herein, the number of couplexes can be determined by analyzing the co-localization of the label of a binding agent of a first type with the label of a binding agent of a second type within one partition or emulsion droplet using nucleic acid amplification, e.g. PCR or dPCR, and e.g. probes or primers labelled with fluorescent dyes and/or alternatively using next generation sequencing (NGS).

In one embodiment the present invention relates to a method for processing a sample, the method comprising the steps of:
A. providing antibodies with uniquely identifiable and amplifiable labels, two antibodies per target analyte (i.e., binding agents of a first type and second type), said labels comprise an identifier sequence (barcode) and, optionally, a unique molecular identifier (UMI).
B. determining binding characteristics of said antibodies including determining the labeled ratio of antibodies, the label synthesis error, also the active fraction of the antibody, and the contamination with free labels and based on these measures determining the apparent dissociation constant;
C. providing a sample containing a concentration of cells, containing target analytes, such as DNA, RNA, proteins, epiproteins, or interacting proteins;
D. optionally, permeabilizing said cells to render the cells penetrable for macromolecules (in particular antibodies);
E. combining the antibodies with determined binding characteristics with said sample forming a binding reaction between said antibodies and said cells including said target analytes;
F. first compartmentalizing single-cells into a plurality of first partitions;
G. combining said compartmentalized single-cells, with buffer having lysing conditions;
H. second compartmentalizing single-target analytes of said target analytes and the formed antibody-target analyte complexes (couplexes) of said individual single-cells into a further plurality of second partitions;
I. optionally, converting RNA into cDNA, concurrently during steps E-H;
J. amplifying said antibody labels, thereby optionally linking the antibody labels of two antibodies forming couplexes with a target analyte, and optionally, said DNA and said cDNA;
K. determining nucleic acid sequence information;
L. identifying from the sequence information determined in step K., the barcodes of said antibody labels and thereby the identity of said target analytes of said single-cells that were bound by said two antibodies to form a couplex, and optionally, determining the composition of first partitions by identifying sequence information of linked antibody labels, based on their UMIs;
M. optionally, determining from the sequence information the RNA composition of said single-cells;
N. determining the absolute amount of said target analytes in single-cells by taking into account the in step B. determined binding characteristics of said antibodies, and optionally determining the chemical compositions of first partitions based on the in step L. identified linked antibody labels in second partitions, wherein the determined chemical composition preferably comprises the amount, concentration or presence of the target analyte in said first partitions.

Preferably during step G. the incubation between contents of a cell and the binding agents is still possible/still occurs, such that binding agents may also come into contact (and form couplexes) with target agents comprised within the cell, e.g., within organelles such as e.g., the nucleus, that were before cell lysis not (or less) permeable and/or accessible to binding-agents.

In some embodiments the step K. of identifying the sequence information comprises collecting sequence information of linked antibody labels using a compartmentalized bi-component assay.

In one embodiment the present invention relates to a method as described herein, wherein the step of combining said compartmentalized single-cell, with buffer having lysing conditions comprises exposing the single-cells to lysing conditions with cell-specific identifiable barcoded amplification primers to amplify said linked labels and optionally, DNA and cDNA, in the step of amplifying the linked labels, and optionally, said DNA and said cDNA, during second compartmentalization to include single-cell identifiable barcoding. In other words, wherein said single-cells are exposed to lysing conditions in step G (as disclosed above) with cell-specific identifiable bar-coded amplification primers to amplify said linked labels and optionally, DNA and cDNA in step J (as disclosed above) during second compartmentalization to include single-cell identifiable barcoding.

In one embodiment the present invention relates to a method, wherein prior the lysing conditions applied to the single-cells in the step of combining said compartmentalized single-cell, with buffer having lysing conditions, chemical treatments are applied including protein cross-linking stabilizing protein interactions, inhibitors preventing deterioration of sample or said labeled antibodies.

In one embodiment the present invention relates to a method, wherein the number of applied antibodies in the step of combining the antibodies with determined binding characteristics with the sample, is advantageously below 10 and the method determining the sequence information of the nucleic acid labels of the binding agents is a compartmentalized PCR bi-component method similar described in WO 2016/083793 A1, applying for steps from the steps of the second compartmentalization to the step of optionally determining from the sequence information the RNA composition of said single-cells. In other words, wherein prior said lysing conditions applied to said single-cells in step G. (as disclosed above) chemical treatments are applied including protein cross-linking stabilizing protein interactions, inhibitors preventing deterioration of sample or said labeled antibodies.

In one embodiment the present invention relates to a method, wherein the number of said antibodies in step E are, advantageously, above 10 and the method determining the sequence information of the nucleic acid labels of the binding agents is a compartmentalized NGS bi-component method, similar to the one described in WO 2016/083793 A1, applying for steps form H to M (or L). In other words, wherein the number of applied antibodies in step E (as described above) are, advantageously, below and the method determining the sequence information of the nucleic acid labels of the binding agents is compartmentalized PCR bi-component method similar described in WO 2016/083793 A1 10 applying for steps form H to M (as described above).

In one embodiment the present invention relates to a method, wherein a plurality of said single-cells are subjected to said method and information gained from said single-biomolecules, said identity and said absolute amount of said antibody recognizable biomolecules, said for plurality of single-cells is determined form information of steps from M (as described above; comprising determining from the sequence information the RNA composition of said single-cells), optionally combined with information of said RNA content of said single-cells.

In some embodiments the first compartmentalization preferably provides chemical conditions that allow carrying out other detection methods including detecting a plurality of RNA molecules. Such RNA detection methods for single-cells are known in the art.

In embodiments targets or target analytes can comprise many epitopes and can bind a plurality of binding agents. In one preferred embodiment all bound binding agents are pairwise detected by using a method of WO 2016/083793 A1 or WO 2020/260277 A1.

The method of the invention is preferably carried out under physiological conditions so that the targets can be detected in their original context i.e., in the same conditions as present in the living cell. This provides information on the targets that occur naturally.

The method preferably applies a first compartmentalization. The chemical compositions of the first compartmentalization is partially unknown, however, deducible from the second compartmentalization. Maintaining the quantitative correspondence the first the second absolute between t compartmentalization is a preferable embodiment of the invention. The first compartmentalization is, preferably, a binding reaction between binding agents and targets of single-cell. In embodiments in the first compartmentalization the concentration of the targets is preferably unknown. Additionally, in embodiments either the concentration of binding agents are unknown, or the concentration of single-cell is unknown, or the volume of the first compartmentalization is unknown. During the second compartmentalization in embodiments the method determines the counts of binding agents and the counts of couplexes corresponding to the first compartmentalization. From the numerical conditions of the method and the determined counts of the first compartmentalization, one can derive the concentration of binding agents and the concentration of couplexes to determine the chemical compositions of the first compartmentalization.

The first compartmentalization preferably provides chemical conditions, concentrations of targets of single cells and binding agents facilitating binding reactions. Such conditions, for example, can be provided by many single-cells in a larger volume or small number of single-cells in smaller volume.

The step of contacting the binding agents with the target is usually carried out in known buffer systems, for example in buffer systems that have already been used for studies of targets of binding agents. The reaction can be carried out at room temperature or 4° C. or under other suitable conditions. To get reproducible signals optimal time, optimal temperature and other assay conditions are determined including the steps of binding and detection. Optimal conditions can be determined by the person skilled in the art, but especially optimized in regards of fixation, permeabilization, incubation times.

Present invention enables the detection of absolute quantities of targets of single-cells at previously undetectable levels, down to 100, 10 or 0 copies of targets per single-cells.

A varied concentration of the binding agent and target (variable number of single-cells) can be used to calculate quantitative parameters for a target, such as protein-protein interactions, proteins and epiproteins (modified/PTM-proteins). Thus, in preferred embodiments the method is carried out using different concentrations of the binding agent and/or target.

In another preferred embodiment during the first compartmentalization couplexes are formed.

In connection to the second compartmentalization the couplexes need to be separated, i.e. isolated from other couplexes prior to linking the label sequences i.e. the associated unique nucleotide sequences. The separation is carried out by methods known in the art. For example, separation can be carried out by dilution, specific binding, or separated by physical and/or chemical properties. Preferably, said dilution is limited dilution. The couplexes are separated into second compartments, such as emulsion droplets, micro-cavities etc., preferably diffusion limited or separated compartments as described in WO 2016/083793 A1.

In a preferred embodiment the binding agents, as well as the target analyte are non-immobilized and in solution, such that the couplexes likewise form in solution.

Preferably the two analyte-specific binding components as well as the analyte are non-immobilized and in solution, such that the bi-component/analyte complexes likewise form in solution. In some embodiments the method comprises a step of providing a sample with a known concentration of analyte. In some embodiments the method is an in-vitro method, wherein preferably a step of providing a sample does not comprise a surgical or invasive treatment of a living human or animal body.

Preferably, said separation comprises any one or more of solid surface binding, dilution or phase separation among the others, or more preferably providing diffusion limited or separated compartments/partitions. The separation limits the number of unbound binding agents per compartment. For example, the mean number of unbound binding agents in a compartment is one or lower, or the mean number of unbound binding agents is a compartment is between one and three. The compartmentalization (e.g., effective separation or isolation of large number reactions) follows the Poisson distribution. Separating the couplexes sufficiently prior to further analysis will provide the circumstances where (optionally linked) pairs of nucleic acid labels are generated, which are based on co-localization of binding agents. Separation reduces the number of non-specific co-localizations of nucleic acid identity labels and allows the identification of specific binding partners especially when complex protein mixtures are investigated.

Separation may result, on average, in preferably a single couplex per compartment, where linking will provide only linked nucleotide sequences of the binding agents, consequently reducing the possibility of random linking between other members of the binding agents. Such separation is preferable for the counting of binding agents/target complexes in the compartments and allows the determination of their absolute amounts, based on the Poisson distribution, however mathematical concepts exist to fully compensate other situations regarding the other end-states of separations.

Separation may result, on average, in a single unbound binding agent per compartment, where linking will provide only self-linked nucleotide sequences, consequently reducing the possibility of random linking between other members of the binding agents. Such separation enables the counting of binding agents in the compartments and allows the determination of their absolute amounts, based on the Poisson distribution, and well-known in the art.

In embodiments generating linked nucleotide sequences in compartments (partitions), amplification methods can be used. Compartmentalized amplification methods are well known to the person skilled in the art, for example emulsion polymerase chain reaction (Schütze et al., Anal. Biochem. 2011 Mar. 1; 410(1):155-7).

In embodiments, generating linked nucleotide sequences in compartments, the amplification is used to create a covalent linkage between the labels and/or to determine the co-localization-based linkage of labels without covalent linkage. In embodiments generating linked nucleotide sequences in compartments or partitions by amplification can comprise linking UMIs of labels to create a uniquely linked sequence or simply a unique sequence (comprising a unique combination of two label sequences).

As demonstrated herein, in preferred embodiments an absolute quantification method for single-cell analysis is provided that may be applied, for example, as a novel single-cell method for proteomics and/or interactomics and related fields, and even nucleic acid analysis (basically any target that may be recognized by binding analytes according to the invention), the scope of invention is generally known for persons skilled in the art. This method can be used for example, for single cells after different separation methods including fluorescence activated cell sorting (FACS), magnetic-activated cell sorting (MACS), laser capture microdissection (LCM), manual cell picking/micromanipulation, different microfluidic platforms and on-demand optically intercepted and controlled single-cell printing (A. Gross, J. Schöndube, S. Niekrawitz, W. Streule, L. Riegger, R. Zengerle, P. Koltay, Single-Cell Printer: Automated, On Demand, and Label Free, J. Lab. Autom. 18 (2013) 504-518. https://doi.org/10.1177/2211068213497204.), enabling highly sensitive, absolute quantitative and parallel proteomic analysis of targets of single-cells. The methods of invention also can be combined to detect cellular mRNA. The method described herein allows better understanding of cell-to-cell heterogeneity due to broader detection of different molecular species in single cells.

The present methods also find use in generating functional quantitative information of the cell's workings, which can be helpful in determining functional differences emerging between different cell types, or diseased states or species between any cellular objects having any function differences.

The novelty of the invention is enabling in preferred embodiments the characterization of binding agents, which can then be used to detect targets of binding agents by applying the determined characteristics of binding agents to analyze single-cells, which have previously been contacted by said binding agents in suspension. In suspensions of cells both the advective transport rate and the diffusive transport rate influence bindings in disperse solutions. In suspension of cells the exerted concentration of targets can be increased to a level of saturated binding agent binding, and from the characterization of binding agents said saturation can be proved and the absolute amount can be derived. Preferably, binding agents' bindings below the saturation level, as well, can be used with the characterization of binding agents and the absolute amount can be derived. Thus, the methods described herein are also suitable for generating quantitative proteomic and/or interactomic or even genomic and/or transcriptomic data corresponding to any binding agent recognizable targets of interest and are not limited to highly abundant target analytes, such as highly expressed proteins or RNA.

The novelty of the invention resides, for example, in a novel quantitative assessment of antibody targets in single-cells. In preferred embodiments, the invention applies two major improvements over the art, (i) determining binding characteristics of labeled binding agents (e.g. antibodies) including considering the labeled ratio of binding agents, the label synthesis error rendering the label undetectable, also the active fraction of the binding agents, which has the capability of binding and the contamination with free labels, preferably using cellular target material of unknown quantity, and based on these measures determining the apparent dissociation constant of the binding agents providing precise, high sensitivity, down to zero targets of measurement of targets of single-cell, and (ii) performing a double compartmentalization of single-cells enabling the absolute quantitative correspondence between conditions of first compartmentalization of single-cells during the binding incubation and the second compartmentalization of single-cells determining the chemical compositions (e.g. the presence or absence of targets) of first compartmentalization by using the determined binding characteristics of the binding agents to determine extremely low, 100 s or 10 s copies per targets per single-cell and absolute amounts of the targets of the binding agents.

The compartmentalization enables having a multitude of separated, uniform and specific conditions.

A preferred embodiment of the first compartmentalization (into first partitions) comprises a suspension of single-cells, wherein the compartmentalization is diffusion-limited and the cells are in their own concentration environment.

Also, preferably, the compartmentalization achieves a high overall concentration (enrichment) of the targets through isolation of single-cells, such that individual single-cells represent a locally different concentration.

In another preferred embodiment of the first compartmentalization (into first partitions) a few cells or single-cells are comprised within an advantageously small volume wherein compartmentalization is physical. Preferably, the compartmentalization achieves a high overall concentration of the targets through encapsulation of single-cells in preferably small volumes, such volumes are in the nanoliter range or in the picoliter range. Methods of encapsulating cells in such low-volume compartments is known in the art, including, for example, droplet microfluidics, microwells and hydrodynamic trapping (Murphy, T. W., Zhang, Q., Naler, L. B., Ma, S. & Lu, C. Recent advances in the use of microfluidic technologies for single cell analysis. Analyst 143, 60 (2018)).

In one preferred embodiment after the exposure of the single-cells to the labeled binding agents (e.g. antibodies or aptamers), preferably during the first compartmentalization (into first partitions), the separation of single-cells is maintained, such that the initially compartmentalized single-cells can be analyzed separately.

The second compartmentalization (into second partitions) facilitates the determination of the chemical composition (herein preferably the concentration of the binding agents and couplexes) of each single cell, that were partitioned during the first compartmentalization by subsequently applying the (apparent) binding characteristics of the binding agents, such that the absolute amounts/concentrations of the target(s) can be derived.

For example, such methods may be used to detect targets below 100 copies or 10 copies per cell as described in Example 1. The methods also find use in generating functional quantitative information of the cell's workings, which can be helpful in determining functional differences emerging between cell types, diseased states or species between any cellular objects having any function differences.

Reactivity of binding agents, such as antibodies, can be described by the on and off rates of the binding agents and their derivatives, and is in the art also known as ‘dissociation constant”. While reactivity is generally considered as a constant related to the chemical nature of the binding agent, e.g., antibody, the value of the dissociation constant is a function of the chemical environment including presence and amount of salts, hydronium ion (pH) and other chemical moieties. Also, changes of the structure, conformation of epitope also alters the dissociation constant of binding agents, e.g., antibodies, and as it is, in most cases, also a part of a macromolecule, epitopes are also a subject of the same effects of the chemical environment. Methods exist to determine the dissociation constant of e.g., antibodies in a given chemical environment to compensate for these effects, including NMR spectroscopy, equilibrium dialysis, dynamic light scattering, analytical ultracentrifugation, ultrafiltration, electrophoretic methods, differential scanning calorimetry, homogeneous time-resolved fluorescence, fluorescence correlation spectroscopy/fluorescence cross-correlation spectroscopy, spectroscopic assays, affinity capillary electrophoresis, biolayer interferometry, dual polarization interferometry, static light scattering and microscale thermophoresis (Wilkinson K D. 2004Quantitative analysis of protein-protein interactions. Methods Mol. Biol. 261, 15-32.doi:10.1385/1-59259-762-9:015).

In embodiments determining the dissociation constants of binding agents, such as antibodies, the compartmentalized bi-component methods advantageously can be applied to derive absolute amounts of the formed couplexes (or ternary complexes), facilitating a target external standard free determination of dissociation constant of antibody in a complex chemical environment, also in a native environment. One embodiment is described in Example 3. The method determining dissociation constant of binding agent(s) is affected by additional factors including the labeled ratio of binding agent(s), the label synthesis error, the active fraction of the binding agent(s) and the contamination with free labels, as shown, for example, in Example 4. In such embodiments, only the labeled ratio of binding agent(s) and/or the label synthesis error need to be independently determined to control and compensate for the precision of said compartmentalized bi-component method determining the dissociation constant of binding agent(s). Those who are skilled in the art can devise methods to determine the labeling ratio of the binding agent(s), the label synthesis error of binding agent(s), for example, as described in Example 5.

As shown in Example 4, however, the active fraction of the binding agent(s) and the contamination with free labels will be advantageously confounded in the determination of dissociation constant of antibody. Example 6 shows the determination of dissociation constants of antibodies varying the labeled ratio, label synthesis error and the active fraction of binding agent(s). Preferably, compartmentalized bi-component methods using labeled ratio and the label synthesis error of binding agent(s) determines the apparent dissociation constant of said binding agent(s), thereby providing an advantageous self-compensation inherently for the active fraction of the binding agent(s) and the contamination with free labels to provide the dissociation constant of antibodies for the unbiased determination of absolute quantities in single-cells.

In one embodiment, preferably, both the determination of dissociation constant of antibodies (binding agents) and the determining of the absolute amount of said target analytes in said single-cells are determined using the same compartmentalized/partitioned bi-component method.

In a preferred embodiment the two analyte-specific binding components of the bi-component method as well as the analyte are non-immobilized and in solution, such that the bi-component/analyte complexes (couplexes) likewise form in solution.

In one embodiment, determining the absolute amount of said target analytes, is carried out in the first compartmentalization, where the compartmentalization is diffusion limited, the binding reaction between the cells and antibodies are carried out at diffusion limited concentration, such as shown, for example, in Example 1. The concentration of target proteins (target analytes) of said binding reaction is preferably defined by the volume of the cell suspension containing the cells and the total target protein content of that volume. Under suitable conditions and assumptions, having negligible volume of cells or including the cell volumes in the whole sample volume by making them antibody- or target protein/analyte-permeable, the concentration of the target protein/analyte can be treated as it is in solution, as described in Example 1. Hence, their concentration per cell can, in such embodiments, be assumed from their concentration calculated for the entire sample volume and the numbers of cells and binding agents present in said sample volume. Separating each single-cell and having a suitable sensitive method, the number of targets per cell can be determined from the extrapolated concentration of the target in the suspension volume divided by the number of cells in suspension. Preferably, said suitable sensitive method is a second compartmentalization method which can be used to count single molecules. Preferably, the cells are lysed before the second compartmentalization. Preferably, the extrapolated concentration of the target in the suspension volume is determined using the binding characteristics and finally results in the determination of the absolute amount of the target analyte per single-cell, as described in Example 1.

The present invention discloses a method for determining the absolute amount of target analytes per single cell using a compartmentalization approach. In one embodiment, the binding reaction between cells and antibodies is carried out in the first compartmentalization. The concentration of target proteins (target analytes) in the binding reaction is defined by the volume of compartment and the total target protein content within that volume. By considering the negligible volume of cells or making them antibody- or target protein/analyte-permeable by lysis, the target protein/analyte concentration can be treated as if it were in solution. Single-cell separation, coupled with a sensitive second compartmentalization method capable of counting single molecules, allows the determination of the number of targets per cell by extrapolating the target concentration in the first partioing volume. The method utilizes lysed cells in the first compartmentalization and leverages binding characteristics to extrapolate the target concentration, ultimately enabling the absolute quantification of target analyte amount per single cell, as described in Example 2.

In one embodiment, determining the absolute amount of the target analyte (e.g. antibody-recognizable biomolecules), is carried out in the first compartmentalization, wherein the compartmentalization is physical, the binding reaction between the cells and the binding agents is preferably carried out at high target analyte-concentration exerted by a low volume encompassing each single-cell. Preferably, the cells are lysed during the first compartmentalization. Each first compartment is re-compartmentalized, and a suitable sensitive method is used to count the single target analyte molecules. Preferably, the concentration of the target in the first compartment is determined using the binding characteristics, and finally results in the determination of the absolute amount/number of the target analyte per single-cell, as described for instance in Example 8.

In one embodiment the suitable sensitive method is a homogeneous compartmentalized bi-component method having no washing step to preserve the binding conditions, similar to that described in WO 2016/083793 A1.

In another embodiment, the bi-component method comprises, additionally, absolute quantitative capabilities having predetermined binding characteristics of binding agents, having predetermined said linked-sequence information (of linked label sequences).

In one embodiment the suitable sensitive method is a compartmentalized bi-component method, similar to what is described in WO 1999/043855 A1. Such method uses the sequence information of the nucleic acid labels of two binding agents of couplexes (therein termed (bi-)components) for detecting the absolute amount of proteins per individual single-cell. Such method is suitable to determine its independent parameters as the antibody concentration in the suspension of single cells and the number of bound antibodies in couplexes can be determined per single-cells from the solution-based partitioning data of second compartmentalization

In one embodiment the suitable sensitive method is a compartmentalized bi-component method, similar to what is described in WO 2016/083793 A1 wherein for the readout a dPCR based method using co-compartmentalization based linkage information is applied.

In one embodiment the suitable sensitive method is a compartmentalized bi-component method, similar to what is described in WO 2016/083793 A1 wherein for the readout a NGS sequencing-based method is applied. In such a method the co-compartmentalization-based linkage information of the nucleic acid labels of the binding agents is based on the determination of linked unique molecular identifiers (UMI), see also present Example 10.

In one embodiment the suitable sensitive method is a compartmentalized bi-component method, that applies unique molecular identifiers (UMI), wherein the UMI sequence comprises at least 2 nucleotides or alternatively, more than 2 nucleotides, or alternatively, at least 4 nucleotides, or alternatively, at least 6 nucleotides, or alternatively, at least 8 nucleotides, or alternatively, at least 10 nucleotides, or alternatively, at least 12 nucleotides, or alternatively, at least 14 nucleotides, or alternatively, at least 20 nucleotides, or alternatively, at most 8 nucleotides, or alternatively, more than 8 nucleotides, or alternatively, at most 10 nucleotides, or alternatively, at most 14 nucleotides, or alternatively, at most 20 nucleotides. UMI sequences have been described in the art, such as by Kivioja et al. (Kivioja et al., 2012, Nat Methods 9: 72-74). In a preferred embodiment UMIs are used in such a way that the molecules incorporating the UMIs are made capable to encode and the encoded information has a quantity. The UMI sequence is a random sequence which may be added to other sequences.

In one preferred embodiment, determining the absolute amount of said antibody recognizable biomolecules of said single-cells is combined with a method for determining the plurality of RNA molecules of single-cells as in Example 7.

In one embodiment the suitable sensitive method detecting said absolute amount of proteins per individual single-cells is combined with the detection of RNA. Methods of RNA-analysis are known in the art, and preferably comprises the use of nucleotide sequences that are capable of hybridizing to nucleic acids, namely that are designed to hybridize to the poly-A tail of mRNA, such as a poly-T sequence. In some embodiments, first strand synthesis is primed with an (anchored) oligo dT primer (or potentially with a randomer or a combination of the two) that is appended with a UMI, an amplification primer binding site, and optionally a template switch primer sequence.

In embodiments of the present invention nucleic acid sequence barcodes may preferably be used to identify labels, molecules or binding agents stemming from single cells. Barcodes ca be identified during the analysis of nucleic sequence information. Unique barcode sequences also allow data to be associated with individual cells.

In addition, or alternatively, the processed RNA may also comprise an UMI. Hence, the sequence information of the nucleic acid labels of the binding agents comprises UMIs that might be linked and/or the processed RNA may comprise at least one of a barcode and an UMI. Thus, in some embodiments, the oligo dT or random hexamer priming is appended with an UMI. Also, in some embodiments the template switching oligo also comprises an UMI.

In one embodiment of the invention, the sample, preferably is a single cell or isolated nucleus. The invention is not limited to any specific cell type. Preferably, the cell is a nucleated cell. Preferably, the cell is a mammalian cell, preferably a human cell.

In the literature many variations of cDNA synthesis for single-cells exist and are well known for those skilled in the art and can be applied here.

In embodiments where the protein, protein interaction and/or posttranslational modified protein detection according to the invention is coupled with RNA detection, the readout/analysis is preferably carried out by next generation sequencing (NSG), such as offered by Roche, Illumina and Applied Biosystems, or also referred to in the art as third generation sequencing, as described by David J Munroe & Timothy J R Harris in Nature Biotechnology 28, 426-428 (2010) or such as offered by Pacific Biosciences and Oxford Nanopore Technologies.

In one embodiment, the method of the invention further comprises depleting the ribosomal RNA (rRNA).

The person skilled in the art straightforwardly understands that there can be additional or alternative methods to amplify the cDNA obtained and the methods applicable here are not limited to polymerase chain reaction (PCR).

In different steps of embodiments of the method as specified herein, there may be one or more adapters ligated to the sequences. In addition, or alternatively, additional nucleotide sequences may be added during the reverse transcription and/or amplification steps by incorporating these sequences in the primer used for respectively reverse transcription and/or amplification.

In an embodiment, the method of the invention further comprises a step of adding single-cell-specific or sequencing method-specific adapters or barcodes.

In an embodiment, the sequences obtained during the readout according to the method of the invention e.g., linked sequences and/or cDNA molecules obtained by the method of the invention, are flanked by one or more universal sequences. A universal sequence that may be present in different members of a plurality of nucleic acid molecules and can allow the replication or amplification of multiple different sequences using a single universal primer that is complementary to the universal sequence. In embodiments the universal sequences may comprise a binding site for a sequencing primer, preferably a binding site for a NGS or deep-sequencing primer.

Embodiments and features of the invention described with respect to any embodiment of the method according to the invention are considered to be disclosed with respect to each and every other aspect of the disclosure, such that features characterizing one embodiment of the method, may be employed to characterize any other suitable embodiment of the method. The various aspects of the invention are unified by, benefit from, are based on and/or are linked by the common and surprising finding that the present method enables the absolute quantification of target analytes on a single cell level.

### DETAILED DESCRIPTION

The invention provides a method for quantitative assessment of binding agent-targets in single-cells. The invention preferably applies two major improvements over the art, (i) using bulk, solubilized target material to determine binding characteristics of binding agents, e.g., antibodies, including considering the labeled ratio of binding agents, the label synthesis error rendering the label undetectable, also the active fraction of the binding agent, which has the capability of binding and/or the contamination with free labels, and determining, based on these measures, the apparent dissociation constant of the binding agent, thereby providing a precise and high sensitivity (down to 10 s targets of single-cells) measurement of target analytes, and (ii) applying double compartmentalization of 1. single-cells and afterwards 2. their contents, thereby enabling the including absolute quantitative correspondence between conditions of first compartmentalization of single-cells (in first partitions) during the binding incubation of binding agents, and the second compartmentalization of single-cell-contents (in second partitions) to enable determining the chemical compositions of the binding agent-binding reactions while using the determined binding characteristics of the binding agents to determine absolute amounts of target analytes, even extremely low amounts.

Unless otherwise defined, technical and scientific terms used herein have the same meaning as commonly understood by one of the ordinary skilled in the art to which this disclosure pertains. One skilled in the art will recognize many methods and materials similar or equivalent to those described herein that could be used in the practice of the present invention. In fact, the present invention is in no way limited to the methods and materials described.

The practice of conventional techniques in cell culture, biochemistry, molecular biology, recombinant DNA, computational biology, bioinformatics, genomics, proteomics, interactomics, sequencing, and related fields is well known to those skilled in the art and is discussed, for example, in the following references: (Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.)

Singular form terms include plural referents unless the content clearly dictates otherwise. Thus, for example, reference to ‘a single-cell’ includes cases of one, two or more cells, and the like, when appropriate.

As used herein, the term ‘linking’ is used to describe and account for any useful linking methods, providing linked co-compartmentalization information gained about the labels of binding agents, linking of the unique nucleotide sequences of binding agents, in said bi-component methods. The term ‘linking’ is used broadly and accounts for physical and no-physical linking methods and it is identical with term ‘linkage information’, or ‘linked sequence information’.

As used herein, the term ‘approximately’ is used to describe and account for small variations. For example, the term may refer to less than or equal to 10, such as less than or equal down to 1, when appropriate, also the term may refer to more than or equal to 10, such as more than or equal up to 100 or more, when appropriate. It is to be understood that range format is used for the sake of simplicity and brevity and is to be flexibly understood to include numeric values expressly stated as boundaries of a range, encompassing each numeric value and sub-ranges.

Herein ‘taking into account’ may preferably refer to the inclusion of one or more parameter into a calculation. In other words, depending on the respective value, factor or parameter to be determined taking into account can refer to the inclusion of certain parameter(s) into a respective or applicable calculation used to determined said value, factor or parameter. In embodiments taking into account may comprise the offsetting of parameters or values with each other. Examples of parameters taken into account for calculating a certain value or constant are provided herein and in the examples. For example, in embodiments the volume of the first compartmentalization (first partition) and/or the initial concentration of cells in the initial solution are taken into account (used in the calculation) during the calculation/determination of the absolute amount/concentration of the target analyte in a sample or cell. Or, for example, in embodiments to determine the labeling efficiency of a binding agent, e.g., an antibody, the concentration [ng/μl] of the light chain, of the heavy chain and the labeled heavy chain are taken into account (used in the calculation) as described herein.

In embodiments pooled droplets/second compartments may be analyzed using nucleic acid sequencing, such as Next generation sequencing (NGS). NGS results may be analyzed according to the procedure described in (PCT/EP2020/067493), briefly, in embodiments, bevor sequencing the ‘compartmentalized’ dPCR may be used to amplify binding agent labels comprising unique labels (UMIs), thereby obtaining—in case of couplexes—nucleic acid sequences comprising two joined label sequences. During NGS data analysis the label sequences comprising two or more UMIs, e.g., a ‘dimerized UMI’, may in embodiments be deconvoluted to reconstruct the content of each droplet (in which the ddPCR amplification of the labels was performed, preferably only comprising one couplex). Namely, in embodiments the evaluation of the binding-reaction may be based on the NGS reading of the binding agent-specific ‘dimerized’ UMI labels generated according to the standard protocol of emulsion coupling (dPCR-based linking two or more binding agent labels in a couplex). In embodiments the number of (labelled) binding agents, e.g. antibodies, in each droplet (preferably forming a couplex) may be determined by counting all unique UMI labels for a given binding agent, e.g. antibody (counting restricted to a given binding agent-specific sequence). In embodiments possible PCR or sequencing duplicates may be discarded during analysis using duplicate ‘dimerized’ label sequences. Couplexes may in embodiments be counted based on their dimerized labels (comprising two or more UMIs)).

In embodiments, once the droplet content has been identified, a Poisson-based calculation (as described in EP 22161450.6) may be carried out to determine the number of detected couplexes. Briefly, the distribution of binding agents during compartmentalization is governed by Poisson distribution, thus counting the occurrences of each binding agent (by the determination of their relative abundances of binding agents by NGS after droplet-based PCR amplification), if the number of droplets are known, the background detection of couplexes can preferably be calculated. A multitude of specific binding events will be used to identify the exact target agents of the binding agents. E.g., in multi-protein complexes the information contained in the linked labels of binding agents is based on the co-localization of respective binding agents on said complex, and indicates direct interaction of detected target analytes with each other. The obtained results may in embodiments be further normalized using background signals and/or the expected Poisson-distribution of single binding agents and their labels.

In other words, in embodiments the linking reaction, e.g., amplification, of labels of binding agents that are comprised within a single couplex, which is itself isolated preferably within a second compartment (droplet), results in linked nucleic acid labels, which comprise the joined nucleic acid sequences of each of said respective labels. For example, if two binding agents bind a target agent forming a couplex, the linked labels comprise the joined nucleic acid sequence of both binding agent labels. Hence, preferably said joined label sequence comprises at least two binding agent type-specific sequences (one from each binding agent) and at least two UMIs, that are each unique to their biding agent molecule und unique within the plurality of binding agents used in said experiment. In embodiments the nucleic acid sequence of said joined (‘dimerized’) labels be identified using nucleic acid sequence analysis (e.g., using NGS). The data of the sequence analysis can then be analyzed for such sequences comprising linked label sequences.

Therefore, in embodiments using nucleic acid sequence analysis, linked label sequences can be identified and wherein each unique combination of at least two labels (within one sequence, comprising a unique combination of at least two different UMIs) is indicative of a couplex formed. Therefore, in embodiments during sequencing analysis each unique sequence comprising at least two label sequences can be counted and the total count (of unique linked label sequences) is preferably equivalent to the total count of couplexes detected/analyzed. In embodiments using the herein described binding characteristics the absolute concentration of a target analyte can be calculated from said count of couplexes. In embodiments determining the absolute concentration of the first target analyte per cell in the first partition is performed by offsetting with each other (taking into account) the initial concentration of the cells in the sample in step a., the binding characteristics determined in step b. and the number and/or concentration of couplexes and binding agents in step h. in the second partition.

In embodiments the dilutions of the samples can be measured in the same sequencing reaction using sample-specific DNA barcodes (e.g. barcoded primers) and as different types of binding agents have distinguishable labels, many measurements (using different antibody pairs against different antigens) may in embodiments be carried out in parallel (multiplexed).

Terms that link statements refer to a situation in which one or more of the cases mentioned can occur alone or in combination with at least one of the cases mentioned up to all cases mentioned, including such terms as ‘comprising’ meaning the construction as being inclusive and open ended, and not exclusive.

Amplifying can refer to a variety of amplification reactions, including but not limited to polymerase chain reaction (PCR and its many variations—Saiki, R. et al. (1985) Enzymatic amplification of beta-globin genomic sequences and restriction site analysis for diagnosis of sickle cell anemia. Science 230, 1350-4., Saiki, R. K. (1988) Primer-directed enzymatic amplification of DNA with a thermostable DNA polymerase. Science 239, 487-91.), especially fluorescent readouts (Holland, P. M. et al. (1991) Detection of specific polymerase chain reaction product by utilizing the 5′→3′ exonuclease activity of Thermus aquaticus DNA polymerase. Proc. Natl. Acad. Sci. USA 88, 7276-80, other references: https://www.promega.de/resources/guides/nucleic-acid-analysis/pcr-amplification/). Isothermal amplification reactions including loop-mediated isothermal amplification (Notomi T, Okayama H, Masubuchi H, Yonekawa T, Watanabe K, Amino N, Hase T (2000). ‘Loop-mediated isothermal amplification of DNA’. Nucleic Acids Res. 28 (12): 63e-63. doi:10.1093/nar/28.12.e63. PMC 102748. PMID 10871386 and U.S. Pat. No. 6,410,278, Notomi T, Hase T, ‘Process for synthesizing nucleic acid’, published 2002 Jun. 25, assigned to Eiken Kagaku Kabushiki Kaisha), whole genome amplification techniques (Zong C, Lu S, Chapman A R, Xie X S. Genome-wide detection of single-nucleotide and copy-number variations of a single human cell. Science. 2012; 338:1622-6. Pmid: 23258894, Klein C A, Schmidt-Kittler O, Schardt J A, Pantel K, Speicher M R, Riethmüller G. Comparative genomic hybridization, loss of heterozygosity, and DNA sequence analysis of single cells. Proc Natl Acad Sci USA. 1999; 96:4494-9. Pmid: 10200290, Dean F B, Hosono S, Fang L, Wu X, Faruqi a F, Bray-Ward P, et al. Comprehensive human genome amplification using multiple displacement amplification. Proc Natl Acad Sci USA. 2002; 99:5261-5266. Pmid: 11959976, Langmore J P. Rubicon Genomics, Inc. 2002; 3:557-560, Telenius H, Carter N P, Bebb C E, Nordenskjold M, Ponder B a, Tunnacliffe a. Degenerate oligonucleotide-primed PCR: general amplification of target DNA by a single degenerate primer. Genomics. 1992; 13:718-725. Pmid: 1639399, Zhang L, Cui X, Schmitt K, Hubert R, Navidi W, Arnheim N. Whole genome amplification from a single cell: implications for genetic analysis. Proc Natl Acad Sci USA. 1992; 89:5847-51. pmid:1631067) strand displacement amplification (G. T. Walker, M. C. Little, J. G. Nadeau and D. D. Shank (1992) Proc. Natl. Acad. Sci 89, 392-396), helicase-dependent isothermal DNA amplification (Vincent M, Xu Y, Kong H (2004). ‘Helicase-dependent isothermal DNA amplification’. EMBO Rep. 5 (8): 795-800. doi:10.1038/sj.embor.7400200. PMC 1249482. PMID 15247927.), recombinase polymerase amplification (Euler, Milena; Wang, Yongjie; Nentwich, Oliver; Piepenburg, Olaf; Hufert, Frank T.; Weidmann, Manfred (2012). ‘Recombinase polymerase amplification assay for rapid detection of Rift Valley fever virus’. Journal of Clinical Virology. 54 (4): 308-12. doi:10.1016/j.jcv.2012.05.006. PMID 22683006, Amer, H. M.; Abd El Wahed, A.; Shalaby, M. A.; Almajhdi, F. N.; Hufert, F. T.; Weidmann, M. (2013). ‘A new approach for diagnosis of bovine coronavirus using a reverse transcription recombinase polymerase amplification assay’. Journal of Virological Methods. 193 (2): 337-40. doi:10.1016/j.jviromet.2013.06.027. PMC 7113639. PMID 23811231, Abd El Wahed, Ahmed; El-Deeb, Ayman; El-Tholoth, Mohamed; Abd El Kader, Hanaa; Ahmed, Abeer; Hassan, Sayed; Hoffmann, Bernd; Haas, Bernd; Shalaby, Mohamed A.; Hufert, Frank T.; Weidmann, Manfred (2013). Meng, Xiang-Jin (ed.). ‘A Portable Reverse Transcription Recombinase Polymerase Amplification Assay for Rapid Detection of Foot-and-Mouth Disease Virus’. PLOS ONE. 8(8):e71642. Bibcode: 2013PLoSO . . . 871642A. doi:10.1371/journal.pone.0071642. PMC 3748043. PMID 23977101, Piepenburg, Olaf; Williams, Colin H.; Stemple, Derek L.; Armes, Niall A. (2006). ‘DNA Detection Using Recombination Proteins’. PLOS Biology. 4 (7): e204. doi:10.1371/journal.pbio.0040204. PMC 1475771. PMID 16756388), or nucleic acid sequence-based amplification (Deiman, Birgit; van Aarle, Pierre; Sillekens, Peter (2002). ‘Characteristics and Applications of Nucleic Acid Sequence-Based Amplification (NASBA)’. Molecular Biotechnology. 20 (2): 163-180. doi:10.1385/mb:20:2:163. ISSN 1073-6085. PMID 11876473. S2CID 28712952), rolling circle amplification (Ali, M. Monsur; Li, Feng; Zhang, Zhiqing; Zhang, Kaixiang; Kang, Dong-Ku; Ankrum, James A.; Le, X. Chris; Zhao, Weian (2014). ‘Rolling circle amplification: a versatile tool for chemical biology, materials science and medicine’. Chemical Society Reviews. 43 (10): 3324-41. doi:10.1039/C3CS60439J. PMID 24643375), and the like.

In the context of the present invention PCR reagents and/or reverse transcription reagents and/or RT-PCR reagents comprise one or more of the reagents selected from the group of DNA polymerase enzyme and/or reverse transcriptase (RT-) polymerase enzyme and/or dNTPs and/or amplification primers and/or poly-A primers for reverse transcription of RNA and/or buffer reagents and/or nuclease inhibitor(s) and/or further agents or enzymes. The person skilled in the art is aware of the components of PCR and reverse transcription reagents that are required for a successful amplification and/or reverse transcription of nucleic acids.

Digital polymerase chain reaction (dPCR) is a PCR technique utilizing enhanced amplification efficacy of low volume PCR reactions for the absolute quantification of target nucleic acids. Quantification principle of dPCR based Poisson distribution-based correction of the counts of amplified single molecules and the single molecular sensitivity of the partitioned reaction (Sykes P. J., Neoh S. H., Brisco M. J., Hughes E., Condon J., Morley A. A. Quantitation of Targets for PCR by Use of Limiting Dilution. Biotechniques. 1992; 13:444-449.). Key design parameters of dPCR platforms include the number of partitions, the volume of each partition, the total reaction volume, and the variation in partition volume. The statistical accuracy of dPCR is further influenced by the variability in sample preparation and the rate of molecular dropouts and false positives. dPCR platforms currently lack the sample multiplexing of qPCR while offering unique assay multiplexing capabilities. (Quan P L, Sauzade M, Brouzes E. dPCR: A Technology Review. Sensors (Basel). 2018; 18(4):1271. Published 2018 Apr. 20. doi:10.3390/s18041271).

There are many forms of real-time or qPCR that differ mainly in the detection chemistries used for monitoring the reaction product, e.g., Gelfand et al., U.S. Pat. No. 5,210,015 (‘Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed (Mackay et al., Nucleic Acids Research, 30:1292-1305, 2002).

Regarding ‘absolute quantification’ of the method a unique molecular identifier-based approach is described to gain information from quantitative compositions of single-molecular compartments (WO 2020/260277 A1). The terms ‘absolute concentration”, ‘absolute number’ or ‘absolute amount’ of a target analyte or biomolecule refers to the total or exact concentration, number or amount of an analyte. In other words, absolute quantification determines the numbers or concentration of an analyte in absolute numbers of molecules or copies. On the contrary relative quantification determines fold changes in numbers, amounts or concentration between two samples. Also, on the contrary other quantification methods need external quantification standards to determine the quantity, unlike the method described in the invention, wherein the absolute molecular count in a volume defines the molar concentration of the target without a comparison to external (relative) quantification standard material.

‘Sequencing’ refers to determining the order of nucleotides (base sequences) in a nucleic acid sample. Next generation sequencing, such as exemplified by methods commercialized by Roche, Illumina and Applied Biosystems, Oxford Nanopores, Pacific Biosciences and others is described (David J Munroe & Timothy J R Harris in Nature Biotechnology 28, 426-428, 2010). A common feature of these technologies is determining the sequence of a plurality of single molecules of DNA, effectively quantitatively revealing the composition of a DNA sample ranging from several hundred up to billions of individual reads in a single run. A sequencing reaction necessitates introducing specific DNA sequences, commonly called adaptors, varied by the technology used. The process named library preparation, involving methods of ligation (e.g.), PCR extension or others. Adaptors may include identifier sequences to distinguish between samples. ‘Next generation sequencing (NGS)’ as used herein shall encompass recently developed technologies for the sequencing of nucleic acids that typically allow much higher throughput than the traditional Sanger approach (see Schuster, Next-generation sequencing transforms today's biology, Nature Methods 5:16-18 (2008); Metzker, Sequencing technologies the next generation. Nat Rev Genet. 2010 January; 11(1):31-46. These platforms can allow sequencing of clonally expanded or non-amplified single molecules of nucleic acid fragments. Certain platforms involve, for example, sequencing by ligation of dye-modified probes (including cyclic ligation and cleavage), pyrosequencing, and single-molecule sequencing. Nucleotide sequence species, amplification nucleic acid species and detectable products generated there from can be analyzed by such sequence analysis platforms. Next-generation sequencing can be used in the methods of the invention, e.g. to quantify unique PCR amplifiable DNA labels in order to assess the formation of bicomponent/analyte complexes as described below.

In embodiments the methods as described herein uses compartmentalized bi-component detection methods. As used herein the term “compartmentalized bi-component detection methods” shall preferably refer to bi-component detection methods that compartmentalize the sample after bringing the analyte specific binding components in contact with the analyte to quantify the formation of couplexes. In general, the compartmentalized bi-component detection methods are preferably based upon compartmentalizing a solution to small compartments such that without the formation of couplexes methods the presence of both binding components in the compartment is unlikely and follows a Poisson distribution. For the compartmentalization, different assays can be envisioned. For instance, an emulsion droplet method may be used to form droplets in an emulsion (e.g. water-in-oil), wherein each droplet represents a separate compartment. Compartmentalization can involve locations or physical compartments, or diffusion limited environment.

The term of a “droplet” as used herein preferably refers to an isolated portion of a first fluid surrounded by a second fluid. The first fluid comprises preferably a hydrophilic fluid such as water, an aqueous media, or a buffer, and preferably comprises the sample solution or the one or more dilutions thereof to which the bi-component detections system or other reagents are added. The second fluid preferably a hydrophobic fluid, such as hydrocarbons, silicone oils, mineral oils, organic solvent. Emulsions techniques to compartmentalize sample solutions are well known in the art. In preferred embodiments the compartmentalized bi-component detection method is emulsion coupling, which refers to is a digital assay concept based on the detection of double-labelled (bi-component), individual ternary molecular complexes in emulsion, which may be identified, for example, by droplet digital PCR (ddPCR) or next generation sequencing (NGS).

Preferably the two analyte-specific binding agents as well as the analyte are provided non-immobilized, i.e., in solution, such that the couplexes likewise form in solution. The term bi-component detection method as used herein, thus preferably address a liquid phase formation of couplexes and is distinct from common sandwich immunoassays involving as a solid phase immobilized (primary) capture binding agents as well (secondary) detection binding agents. The term “non-immobilized” thus preferably refers to components, such as the analyte-specific binding components, that may freely diffuse within a (liquid) solution, such that the binding kinetics to the analyte, equally freely diffusing in said liquid solution, are governed by equations for law of conservation of mass and law-of-mass action in solution as described herein.

In preferred embodiments determining the binding characteristics comprises determining for each binding agent of a first and a of a second type: the dissociation constant for the target analyte, the specificity of the binding to the target analyte, the labeled ratio, the label synthesis error, the active fraction, the contamination with free labels and/or the optimal concentration (number of molecules per unit volume) required for binding to the target analyte. Hence, in other words in embodiments the binding characteristics of a binding agent may include (re) activity, specificity and/or concentration of the binding agent. The reactivity can be described by the dissociation constant of the binding agent, specificity described by identifying binding targets and concentration is described by number of molecules per unit volume.

Generally, a dissociation constant (KD) is an equilibrium constant specifying the tendency of a binding agent to reversibly dissociate (separate) from its target analyte. In other words, the dissociation constant may specify the ‘reliability’ and/or strength of a binding agent to bind to a target analyte and facilitate its detection. In embodiments, a bi-component method using the labeled ratio and/or the label synthesis error of binding agent(s) is used to determine the apparent dissociation constant of the binding agent(s). Hence, in embodiments the consideration of a dissociation constant for calculating the absolute concentration of a target agent herein is preferably unlike, and more complex than the consideration of a simple titration curve in the prior art (e.g., in methods for determining a target agent concentration in a sample or cell), as preferably in the context of the present method the labeling ratio and/or label error of binding agents are additionally (independently) determined and used to control and compensate (normalize) the precision of the method for determining the dissociation constant of a binding agent, thereby achieving a higher precision of the absolute quantification of a target agent than it would be possible with any prior art method.

In embodiments a concentration reference curve for the bi-component detection method or a dissociation constant relationship for the bi-component detection method may be provided in form of reference data. In embodiments, reference data for a concentration reference curve for a bi-component detection method shall preferably relate to any data that allows for providing a mathematical function reflecting the dependence of the signal reflecting the formation bi-component/analyte complexes formed in said solution on the concentration of analyte. In embodiments, reference data for a dissociation constant relationship for a bi-component detection method shall preferably relate to any data that allows for providing a mathematical function for the relationship between the dissociation constants of the analyte-specific binding components (kd1 and kd2, respectively) with the analyte in dependence of the signal reflecting the concentration of bi-component/analyte complexes and the concentration of analytes and/or analyte specific binding components.

Compartmentalized generation of ‘linked sequence information’ may in embodiments herein refer to the physical linking of the labels, preferably nucleic acid labels, of two binding agents, if they are co-localized in a couplex within a partition—as they are synchronously binding their target analyte-thereby forming a couplex. In other words, the linking process has the potential to form random multimer nucleic acid products based on co-localization of these nucleic acid identity labels under suitable assay conditions. In embodiments the linking reaction can be amplification-based or involve other techniques. Amplification based linking can utilize two or more amplification primer pairs with identical binding abilities, but with complementary 5′ tags or dimer linker sequences which result in the formation of polymerase extendable nucleic acid duplexes. The tags or dimer linker sequences mean that the sequence amplified by one primer pair will hybridize to sequences amplified by the second primer pair. The identity labels representing the type of binding agents thereby become linked. By physically linking nucleotide labels of binding agents, if they form a couplex with their target analyte facilitates the ‘translation’ of information on the co-localization of binding agents and their targets within a partition to a nucleotide sequence (the physically linked nucleotide labels). In embodiments the identity of the binding agents and therewith the presence of their target may be determined from the nucleotide sequence, which may be achieved in embodiments by nucleic acid sequencing or PCR, e.g., digital (droplet) or real-time qPCR. One embodiment of this process is described in WO 2016/083793 A1. Optionally the linked nucleotide sequences from more than one couplex are combined prior to identifying the linked labels. The identity of the binding agent(s) and/or target(s) can be determined, for example, by sequencing the linked nucleotide label sequences. This can be carried out using a highly parallel system. The linked sequences can be combined so that a single reaction can be carried out to identify all the linked sequences. For example, all of the linked nucleotides can be sequenced in a single reaction. The linked sequences can be determined quantitatively, to measure the relative abundances of the linked sequences. In some embodiments the labels of co-localized binding agents of a couplex are not physically linked, but their co-localization within a second partition, and hence within a couplex, is detected by PCR, preferably dPCR and/or real-time qPCR using a fluorescence detection method. This detection approach of co-localized nucleic acid labels to reliably determine the presence of a couplex, and hence the presence of a target analyte within a second partition, is facilitated, as the distribution of the components of a single cell of a first partition into second partitions follows the Poisson distribution. Therefore, the probability of two binding agents being present in the same second partition, if they are not bound to a target analyte and form a couplex is less than the probability that the two binding agents are co-localized as they are physically connected by binding to a target analyte hence forming a couplex. Hence, in embodiments their nucleic acid labels can be detected by PCR methods, without previously linking the labels physically, but, for example, with two differently labelled (e.g., two different fluorescent colors) probes by droplet PCR.

Accordingly, in this embodiment the presence of two different fluorescence colors within a second partition, e.g., an emulsion droplet, during detection would indicate the presence of a couplex in said second partition. In embodiments where multiple target analytes are analyzed, for each target analyte two specific binding agents, one of a first and one of a second type should be chosen. The differentiation between the different pairs of binding agents for the different target analytes can be achieved using, for example, in some embodiments different fluorescent labels, primers or probes during a final PCR, qPCR, real-time PCR or dPCR readout. In one example a probe that specifically binds to the linked sequence of the two binding agent labels for a first target analyte are tagged with one fluorescent color, while a probe specific for the linked binding agent labels for a second target analyte are tagged/marked with a different fluorescent color. In other embodiments where the labels of binding agents within couplexes are not linked, each pair of binding agents in a couplex is detected by a specific combination of fluorescent colors that can be distinguished during final PCR readout. In embodiments the read-out of the label sequences may be performed by nucleic acid sequencing. In such embodiments the nucleic acid labels of binding agents within couplexes are preferably linked before the readout by sequencing, preferably within the second partitions, e.g., by a ligase reaction or PCR or other means known in the art, followed preferably by a pooling step of all second partitions and a batch sequencing analysis of all labels comprised within partitions. During analysis of the acquired sequence information the linked label sequences can be determined and assigned to single couplexes, hence to single target analyte molecules.

Examples of linked sequences information described in WO 2016/083793 A1 and Karakus et al. (Karakus U, Thamamongood T, Ciminski K, Ran W, Günther S C, Pohl M O, Eletto D, Jeney C, Hoffmann D, Reiche S, Schinkothe J, Ulrich R, Wiener J, Hayes M G B, Chang M W, Hunziker A, Yángüez E, Aydillo T, Krammer F, Oderbolz J, Meier M, Oxenius A, Halenius A, Zimmer G, Benner C, Hale B G, García-Sastre A, Beer M, Schwemmle M, Stertz S. MHC class II proteins mediate cross-species entry of bat influenza viruses. Nature. 2019 March; 567(7746):109-112. doi: 10.1038/s41586-019-0955-3. Epub 2019 Feb. 20. PMID: 30787439., WO 2016/083793 A1) for detecting proteins, epiproteins (PTM-/modified proteins) and protein-protein interactions, and particularly for labeling, analyzing, detecting and measuring protein-protein interactions with below 10 antibodies.

Herein, the ‘ligation’ reaction, may also be referred to as the ‘ligation of labels’, ‘linking of labels’ or ‘linking of binding agent labels’ or ‘linking of antibody labels’. In some embodiments the ligation reaction referred to herein is a classical proximity ligation reaction involving rolling circle DNA synthesis or rolling circle amplification. In other embodiments the ligation is achieved using a ligase enzyme. Non-limiting examples of proximity based bi-component methods comprise methods employing a resonance energy transfer assay, preferably a F6rster resonance energy transfer (FRET) assay or a bioluminescence resonance energy transfer (BRET) assay, a protein complementation assay (PCA), Alphascreen or a DNA labeled proximity assay, preferably a proximity ligation assay (PLA) or a proximity extension assay (PEA). These proximity assay technologies are well known in the art. For further reference on FRET or BRET see e.g. (Pfleger, Seeber, & Eidne, 2006), for a protein complementation assays Morel!, Ventura, & Aviles, 2009, for Alphascreen Taouji, Dahan, Bosse, & Chevet, 2009 and for DNA labeled proximity methods such as PLA (proximity ligation assay) or PEA (proximity extension assay) Soderberg et al., 2006.

As used herein, a ‘barcode’ refers to a nucleic acid sequence that is preferably used to identify the single cells during obtaining the sequence based linked information. The unique barcode sequence allows data to be associated with the individual cells. As used herein, a barcode may also refer to a nucleic acid sequence that is preferably used to identify the single cells during obtaining the sequence based linked information. The unique barcode sequence allows data to be associated with the individual cells.

Processed RNA may also comprise an UMI. Hence, in embodiments the linked sequence information comprises UMIs that might be linked and/or the processed RNA may comprise at least one of a barcode and an UMI. Thus, in some embodiments, the oligo dT or random hexamer priming is appended with an UMI. Also, in some embodiments the template switching oligo also comprises an UMI. In the literature many variations of cDNA synthesis for single-cells exist, and are well known for those skilled in the art and can be applied here.

A specific embodiment of compartmentalized generation of ‘linked sequences information’ using unique molecular identifiers (UMI) and described in WO2020260277A1 where UMIs described at Kivioja at al. (Teemu Kivioja, Anna Vähärautio, Kasper Karlsson, Martin Bonke, Martin Enge, Sten Linnarsson, and Jussi Taipale. Counting absolute numbers of molecules using unique molecular identifiers. Nat. Methods, 9(1):72-74, January 2012.). Various methods have been developed to improve accuracy of quantification of different polynucleotides in a sample using next-generation sequencing technologies, including such methods as competitive polymerase chain reaction (PCR), described in U.S. Pat. No. 5,213,961 and deep barcode sequencing using unique molecular identifiers (UMI), as described by (Smith, A. M., Heisler, L. E., Mellor, J., Kaper, F., Thompson, M. J., Chee, M., Nislow, C. (2009). Quantitative phenotyping via deep barcode sequencing. Genome Research, 79(10), 1836-1842. https://doi.org/1 0. 11 0 1/gr.093955. 109).

‘Nested PCR’ means a PCR, where the product of the first amplification serves as a template for a second amplification PCR reaction. As used herein, said inner primers in reference to a nested amplification reaction mean the primers used to generate a first amplicon, and said outer primers mean the primers used to generate a second, or nested, amplicon.

Multiplexing of PCR means a PCR wherein multiple target sequences are provided and are simultaneously amplified in the same volume (Bernard P S, Ajioka R S, Kushner J P, Wittwer C T: Homogenous multiplex genotyping of hemochromatosis mutations with fluorescent hybridization probes. Am J Pathol 1998; 153:1055-1061). Multiplexing PCR can be combined with dPCR providing having low multiplexing in the compartments and having a large multiplexity of PCR in the overall volume of the dPCR (Alexandra S. Whale, Jim F. Huggett, Svilen Tzonev. Fundamentals of multiplexing with digital PCR. Biomolecular Detection and Quantification. Volume 10, 2016, Pages 15-23, ISSN 2214-7535, https://doi.org/10.1016/j.bdq.2016.05.002).

Herein a ‘target analyte’ or ‘target’ may also refer to a ‘biomolecule’ or an antibody-recognizable biomolecule or simply a ‘target’. Said terms may be used interchangeably herein. Accordingly, these terms may be used interchangeably herein. A target as used herein may be a single molecule or a group of molecules (e.g., at least two molecules forming a complex, such as protein-protein complexes, protein-nucleic acid complexes, substance-protein complexes or substance-nucleic caid complexes) which form a complex with the binding agent(s), comprising non-exhaustively, proteins, modified or post translationally modified (PTM) proteins (epiproteins), including artificial or naturally modified proteins, including phosphorylation, glycosylation, ubiquitination, nitrosylation, methylation, acetylation, lipidation and proteolysis, and protein interactions, non-protein molecules recognizable by binding agents, e.g. nucleic acids or modified nucleic acids. A complex is usually formed under conditions that maintain the conformation of the target of the organism of interest. A target or target analyte may herein be selected from the group comprising peptides, proteins, chemically modified peptides or proteins, nucleic acids, chemically modified nucleic acids, substances and any complexes thereof (any complexes comprising one or more of the enlisted).

Herein a ‘couplex’ describes the complex formed by two binding agents (a pair of binding agents) or analyte-specific binding agents and the target analyte itself, to which the two binding agents specifically bind. A couplex may also be called a ‘bi-component/analyte complex’ or a ‘ternary complex’, preferably in the context of a bi-component detection method, such as the one described in WO 2020/260277 A1. A couplex preferably comprises a binding agent of a first type and a binding agent of a second type, that each bind to different epitopes on the same target analyte molecule. In embodiments the binding agents of a first and of a second type are two different antibodies, that recognizes different isotopes of the target analyte and can bind at the same time to the target analyte. The binding and detection of two different binding agents (of different types) to the same target analyte can in embodiments have numerous advantages over the detection by a single binding agent. For example, in embodiments one type of binding agent may recognize a chemical or post-translational modification and the second type of binding agent may recognize a specific protein. In another embodiment one type of binding agent may recognize a conserved epitope of a protein, while the second type recognizes a specific mutation or variant of a target protein. In another embodiment the target analyte may be a protein complex, or a DNA-protein complex, wherein one type of binding agent recognizes the protein in the complex, while the other type of binding agent recognizes e.g., a methylated DNA motif or in case of protein-protein complexes, each binding agent type recognizes one of the proteins forming a complex. The second compartmentalization step of the present method facilitates the separation of single molecules or molecule-complexes comprised within a lysed cell, according to the Poisson distribution. Thereby the presence of two binding agents of different types in a droplet, bead or partition indicates that said two types of binding agents bind together to the target analyte and form a couplex with it. The (un)likeliness of two binding agents simply being in the same partition by pure chance, but without being in a couplex, can be simply calculated.

In embodiments the present method can be used to identify a plurality of binding agents which bind to a single target. For example, in embodiments where the target is a protein, the method can identify antibodies which bind to different epitopes on the protein. Alternatively, the target can be a protein complex, and the method can identify a plurality of binding agents which bind to different proteins within the complex. For example, in embodiments the nucleic acid label associated with one binding agent in a couplex can be linked to a nucleotide sequence associated with a second binding agent in the couplex. Once the identity of the binding agents is known (from the linked nucleic acid label sequence), it may be possible to identify the components of the target, and thus, for example, the proteins in the target which naturally interact with each other. For example, in embodiments where the binding agent is an antibody with known binding characteristics, the protein bound by the antibody may be identified. Thus, the identity of the proteins within the target can be identified. This allows protein-protein interactions within the sample to be detected and identified. Furthermore, once a protein-protein interaction has been identified, the method can be used to monitor the effect of a compound on the interaction. Alternatively, in embodiments the nucleotide sequence associated with a binding agent in the binding agent/target complex can be linked to a nucleotide sequence associated with a target within the binding agent/target complex. In embodiments, this method can be used to identify which binding agent interacts with which target. For example, it can be used to identify which members of a binding agent library can form a complex with a known target. This information can be used to characterize the members of a binding agent library to gain binding characteristics information. Preferably, in embodiments the production of said random paired, linked nucleic acid products comprises utilizing at least two pairs of PCR primers to amplify identical or non-identical amplicons; wherein optionally the PCR primers at 5′ end have sequence tags wherein amplification with tagged primers results in random paired, linked nucleic acid products. More preferably, amplification is emulsion PCR amplification or digitalPCR (dPCR) and the production of said amplicons and random paired, linked nucleic acid products are parallel processes. Preferably, said sequencing of said joined amplification products is a highly parallel sequencing method (e.g., NGS).

Preferably the ‘binding agent’ is an antibody, aptamer, or based on an engineered protein scaffold. Preferably, the term ‘antibody’ means any binding agent. Alternatively, the binding agent may be a compound. Herein binding agents can be differentiated into different ‘types’, wherein a certain type of binding agent preferably recognizes a specific epitope or target binding motif on a target analyte. Accordingly, different types of binding agents recognize different motifs or epitopes of, or on a target analyte. Hence, preferably two analyte-specific binding agents are used that both exhibit a binding potential for the target analyte. Preferably, the two analyte-specific binding agents do not compete with each other for the binding to the analyte but are chosen and designed to allow for a simultaneous binding of the components to form couplexes. Accordingly, herein two types of binding agents that are specific for one target analyte can bind the target analyte at the same time at different positions or locations of the analyte. Thereby one type of binding agent may specifically bind to a domain, chemical modification (e.g., PTM) or motif of a target analyte, while the other type of biding agent may recognize or specifically bind to another domain, epitope or motif of a target analyte. In cases of molecule complex being a target analyte, the different types of binding agents may recognize different members or parts of said complex, thereby facilitating the specific detection of a complex instead of separate member or part thereof. Such complexes may be protein-protein, protein-peptide, peptide-peptide, protein-nucleic acid, compound-nucleic acid, compound-protein, compound-peptide or compound-compound-complexes, or any complex that might be specifically recognized by a binding agent.

The binding agent may be a member of an antibody display library or a library of antibodies wherein each antibody is labeled with a unique nucleotide sequence. The method may use antibody agents as the binding agent, where the binding characteristics, for example, the target to which the binding agent binds and the chemical properties of binding, including numerical representations of affinity of binding, on and off rates of binding, further including numerical consideration of some or all of the followings labeled ratio, label synthesis error, active fraction, contamination with free labels, are known and the unique nucleotide sequences associated with the plurality of displayed antibody agents are determined and the binding characteristics and unique nucleotide sequences are correlated with one another. In embodiments, when two binding agents, preferably one binding agent of a first and one of a second type, bind to the same target agent molecule, thereby forming a couplex, said couplex (comprising the two binding agents and the target analyte) may also be referred to as a ‘analyte-bi-component complex”. Said two binding agents may, especially in the context of a bi-component detection method, also be referred to as ‘analyte-specific binding components’ or simply ‘binding components”.

The binding agent used in the invention may be an antibody. The term ‘antibody’ as used herein refers to immunoglobulin molecules and immunologically active portions of immunoglobulin molecules, i.e., molecules that contain an antigen binding site that specifically binds an antigen, whether natural or partly or wholly simulated produced. The term ‘antibody’ includes antibody fragments, derivatives, functional equivalents and homologues of antibodies, humanized antibodies, including any polypeptide comprising an immunoglobulin binding domain, whether natural or wholly or partially simulated and any polypeptide or protein having a binding domain which is, or is homologous to, an antibody binding domain. Chimeric molecules comprising an immunoglobulin binding domain, or equivalent, fused to another polypeptide are therefore included. Cloning and expression of chimeric antibodies are described in EP-A-0120694 and EP-A-0125023. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. In addition, fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et ai, Nature 341: 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et ai, Science 242:423-426 (1988); Huston et al, PNAS USA 85:5879-5883 (1988)); (viii) bispecific single chain Fv dimers (PCT/US92/09965) and (ix) ‘diabodies’, multivalent or multispecific fragments constructed by gene fusion (WO94/1 3804; P. Hollinger et ai, Proc. Natl. Acad. Sci. USA 90:6444-6448 (1993)).

An ‘antigen binding domain’ is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

Alternatively, the binding agents may be based on engineered protein scaffolds. Protein scaffolds are derived from stable, soluble, natural protein structures which have been modified to provide a binding site for a target molecule of interest. Examples of engineered protein scaffolds include, but are not limited to, affibodies, which are based on the Z-domain of staphylococcal protein A that provides a binding interface on two of its a-helices (Nygren, P. A. (2008). FEBS J 275(11): 2668-76); anticalins, derived from lipocalins, that incorporate binding sites for small ligands at the open end of a beta-barrel fold (Skerra, A. (2008) FEBS J 275(11): 2677-83), nanobodies, and DARPins. Engineered protein scaffolds are typically targeted to bind the same antigenic proteins as antibodies. Short peptides may also be used to bind a target protein. Phylomers are natural structured peptides derived from bacterial genomes. Such peptides represent a diverse array of protein structural folds and can be used to inhibit/disrupt protein-protein interactions in vivo (Watt, P. M. (2006). Nat Biotechnol 24(2): 177-83)].

Alternatively, the binding agent may be an aptamer. Aptamers are simulated oligonucleotides (DNA or RNA) that recognize target molecules with high affinity and specificity through a combination of shape complementarity and non-covalent chemical bonds (Blank & Blind, Current Opin. Chem. Biol., 2005, 9:336-342). These artificial ligands are quite easy to obtain in vitro and can be developed to recognize a large variety of different molecule classes which range from mere ions (e.g., Pb 2+, Liu & Lu, 2003. J Am Chem Soc., 125, 6642-6643) to nucleotides, small molecules, proteins, viruses, and cells up to whole organisms (Menger et al., 2006. Handbook of Experimental Pharmacology, 359-373). High binding affinity aptamers have been selected through the well-known SELEX method (Ellington& Szostak, 1990. Nature, 346, 818-822) for the detection of low molecular weight molecules like theophyllin (Jenison et al., 1994. Science, 263, 1425-1429), L-arginine (Geiger et al., 1996. Nucl. Acids Res., 24, 1029-1036), moenomycin (Schuerer et al., 2001. Bioorg. Med. Chem., 92, 2557-2563), 17b-estradiol (Kim et al., 2007. Biosens. Bioelectron., 22, 2525-2531) but also for larger molecules like thrombin (thrombin-binding aptamer: 5′-GGTT-GGTGTGGTTGG-3′) (Baldrich et al., Anal Chem. 2004, 76, 23, 7053-63), cholera toxin or HIV-1 tat protein, among others (for review see Tombelli et al., 2007, Biomolec Eng., 24, 191-200). Some of the above-mentioned aptamers have been used in ELISA-like assays on microplates or on the surface of biosensor transducers (QCM, SPR). An aptamer-modified AuNP colorimetric system has also been developed for the determination of the protein PDGF in a sandwich-based assay (Huang et al., 2005, 77, 5735-5741). Recently, modified nucleotide-based aptamer libraries are applied to measure proteins in a highly parallel manner. Aptamer sequences can be tagged with unique sequences or themselves are unique and serves as nucleotide labels of binding agents.

The binding agent may be part of a library, such as a display binding agent library, for example bacterial display, mRNA display, bacteriophage display, aptamer, ribosome display or yeast display libraries. Each member of the library has a detectable, nucleic acid identity label, which is preferably unique to one member of the library. Preferably the unique nucleic acid identity labels are linkable. ‘Linkable’ means the linking process has the potential to form random nucleic acid linkage products based on co-localization/compartmentalization of these nucleic acid identity labels under suitable assay conditions. Preferably the product is a dimer or a pair, or higher order of co-compartmentalized labels without covalent connection.

Preferably, the binding agents are capable of detecting more than one target, preferably with different apparent affinities. Alternatively, the binding agents are capable of detecting a single target using different epitopes or binding sites, preferably with different apparent affinities.

Preferably, any binding agent, which binds to any target and associated with a unique nucleotide sequence is applicable in the invention.

One binding agent can recognize a specific target e.g., the corresponding protein. This is termed specificity. The WO 2016/083793 A1 assay applied here as reference, uses two antibodies per detection enhancing these specificities. Alternatively, a plurality of binding agents may recognize one target, such as a specific target e.g., corresponding protein. This is termed redundancy. Similarly, one binding agent can recognize more than one target, such as a protein species, based on the similarity of the target conformation due to, for example, protein conformation or protein sequences. This phenomenon is termed cross-reactivity. Furthermore, binding agent recognition of a target protein is based on conformation of protein or its protein sequence. This is known as its reactivity and its numerical representation, its affinity is the dissolution constant of the binding agent. Protein binding affinities of binding agents such as can be calculated from the quantitative information of according to WO2020260277A1.

The predetermined binding characteristics of binding agents may include reactivity, active fraction of binding agent, and cross-reactivity with specificity and redundancy and calculated affinities, e.g., apparent dissociation constant of binding agent. Additionally, binding agents are preferably associated with unique nucleotide sequences, and as described in the present invention said associated unique nucleotide sequences modifies the apparent, measurable dissociation constant of binding agents, additionally including as factors of influence the contamination with free labels, labeled ratio of the binding agent and label error.

In embodiments wherein one or more binding agents are antibodies (or fragments thereof), the step of determining the labeling efficiency, comprises calculating the labeled fraction (LE) of binding agents using the concentration of [ng/μl] of the light chain (Ic), the heavy chain (hc) and the labeled heavy chain (L) of an antibody as follows:

$$( 1 - {( \frac{h c}{h c + L} )}^{2} ) * 100 .$$

In embodiments determining the concentration of free labels (labels not bound to binding agents) after the labelling reaction of the binding agent(s) comprises performing nucleic acid amplification, e.g., by dPCR, thereby determining the concentration (e.g., in cp/μl) of the free labels (labels not bound to binding agents) and the concentration of the labeled binding agent (e.g., in cp/μl) in a labelling reaction volume or any other (stock) solution comprising the binding agents, and calculating therefrom the fraction of free labels.

In embodiments binding agent-labels may be amplified using PCT or dPCR (e.g., as described in Example 1) in comparison to a control sample (e.g., a nucleic acid/PCR standard). Preferably dPCR enables to determine the exact amplifiable/detectable amount (concentration or percentage) of the binding agent labels. In embodiments the difference of the two amounts/concentrations (of binding agent vs. control) determined in said measurements can then serve/be used to calculate the label synthesis error.

In embodiments, a bi-component method using the labeled ratio and/or the label synthesis error of binding agent(s) is used to determine the apparent dissociation constant of the binding agent(s). This may preferably provide an advantageous, inherent, self-compensation for the active fraction of the binding agent(s) and the contamination with free labels to provide the dissociation constant of antibodies for the unbiased determination of absolute quantities in single-cells.

In embodiments, calibration curves may be calculated as described herein, for each sample. The calibration curves may be calculated as described in WO 2020/260277 A1, however, with the adjusted binding characteristics. Using the separately determined labeling ratio and/or the label error of the antibodies a corrected calibration curve, e.g., corrected with the corrected values of single and double positive values of dPCR, results in the corrected concentration of antibodies and couplexes. This constitutes a significant improvement over the prior art, where commonly only uncorrected calibration curves may be sued to determine the concentrations of target analytes in a sample. However, the calibration curves of the prior art do not consider any errors or bias resulting from um-labelled binding agents and/or errors in label synthesis etc. This shortcoming of the prior art is surprisingly overcome by the present method, where in embodiments label errors or bias from wrongly labelled binding agents are considered during the quantification of target analytes, in other words are used to normalized the data obtained from, e.g., calibration curves and/or sequence analysis.

In embodiments the apparent dissociation constants of binging agents might be calculated recursively minimizing the standard deviation of the recalculated target analyte concentrations for all measurement under the assumption of that all samples contains the same concentration of target analyte.

In chemistry, biochemistry, medical technologies and pharmacology, a dissociation constant (Kd) is a specific type of equilibrium constant that measures the propensity of a larger complex to dissociate reversibly into smaller components (Friguet, Chaffotte, Djavadi-Ohaniance, & Goldberg, 1985). The dissociation constant is the inverse of the association constant. In the special case of embodiments of bi-component measurement methods, the dissociation constants are the dissociation constants between the two components (binding agents) and the target analyte. As the components can be antibodies, the determination of the dissociation constants has a broad interest. In the prior art some approaches are known for determining dissociation constants.

Accordingly, the term ‘bi-component detection method’ or ‘bi-molecular detection method’ refers to a method using two analyte-specific binding components and determining a signal reflecting the formation of bi-component/analyte complexes (couplexes), when said two analyte-specific binding components are brought into contact with a solution containing the analyte. The term ‘bi-component detection system’ or ‘bi-molecular detection system’, preferably refer to components or reagents necessary to carry out a bi-component detection method. This encompasses preferably two analyte-specific binding components (binding agents), preferably provided in a single or separate solution at a known concentration as well as optional a sample solution containing the analyte to be analyzed.

Preferably, said bi-component detection method is compartmentalized. Said compartmentalization is diffusion limited, physical. Preferably, said compartmentalized bi-component detection method is in solution, or preferably involving surface bound molecular components.

In the bi-component detection methods, preferably two analyte-specific binding components are used that both exhibit a binding potential for the analyte. Preferably, the two analyte-specific binding components do not compete with each other for the binding to the analyte, but are chosen and designed to allow for a simultaneous binding of the components to form bi-component/analyte complexes. As stated previously, the analyte may refer to an aggregate or complex formed of multiple entities, e.g. a protein-protein complex. One of the two analyte-specific binding components may bind to one entity of the analyte complex e.g. a first protein, and the other to a second entity of the analyte, e.g. a second protein. Thereby a bi-component/analyte complex is only form in case of interaction of both proteins, which in turn are labelled with the binding components.

In one embodiment determining the ‘dissociation constant’ of a binding agent may comprise preparing one or more dilutions of the sample or binding agents with known dilution factors and applying a bi-component detection method to the sample as well as the one or more dilutions, wherein applying the bi-component detection method for determining the dissociation constants of analyte-specific binding agents preferably means that two target analyte-specific binding agents (bi-components) at known concentrations are brought into contact with the sample at known concentration in order to produce a signal that depends on the concentration of two-binding agent/target analyte complexes (couplexes or bi-component/analyte complexes) formed in said solution. Either the concentration of the analyte-specific binding agent or the sample is varied by dilution providing dilutions with known dilution factors. The signal detected of in sample and in the one or more dilutions as well as the concentration of target analytes and/or target analyte-specific binding agents can be used as a constraining input for a mathematical fit. The signal can take a variety of forms and depends on the bi-component detection method used. In case of a compartmentalized bi-component method, such as coupling described in WO 2020/260277 A1, the signal may also refer to signals resulting from a digital droplet PCR (dPCR) and/or NGS sequencing that indicate the presence of a bi-component (two binding agents)/target analyte complex (couplex) based upon a deviation from the expected Poisson distribution in the compartmentalized droplets.

‘Emulsion’ herein refers to a water-in-oil emulsion, which is used to compartmentalize single cells or molecules into single emulsion droplets or compartments comprising a very small volume. The person skilled in the art is familiar with various method for generating water-in-oil emulsions, such as in some embodiments microfluidic devices. Droplet-based microfluidics manipulate discrete volumes of fluids in immiscible phases generating (micro) droplets comprising miniature volumes (μl to fl). Alternatively, in other embodiments, partitions may be gel beads in emulsion generated using the gel beads-in-emulsion (GEM)-technology according to, e.g., 10× Genomics.

‘Partition’ herein refers to a said emulsion and also physically separated partitions. The person skilled in the art is familiar with various method for generating partitions, such as in some embodiments microfluidic devices. Alternatively, in other embodiments, partitions may be nanowells, e.g., QIAcuity nanowells, Qiagen.

In the context of the present invention a ‘first partition’ is a partition, compartment or emulsion droplet that comprises one single-cell or no cell. In the present method it is preferred that the sample is diluted and compartmentalized in a fashion that enables single first partitions or droplets to comprise one single cell, or no cell, preferably not more than one cell. This separation and compartmentalization in first partitions is essential to facilitate n analysis on a single-cell level. In some embodiments of the present method each single-cell is lysed inside its first partition to release the components of the cell into the volume of the first partition or droplet and thereby facilitate the subsequent further separation of single molecules of each cell into second partitions. Accordingly, in the context of the present method a ‘second partition’ comprises preferably only one molecule, preferably only one target analyte that is preferably bound to a first and a second type of binding agent, forming a couplex. Accordingly, it is preferred in the context of the present invention that the compounds of a single cell inside a first partition are diluted and separated into second partitions in such a manner that each couplex that was formed is comprised within a separate second partition or droplet. This separation enables the exact determination of the number of individual couplexes formed during the present method. In the context of the present invention a ‘first partition’ may also be termed a ‘first compartment’, a ‘second partition’ may also be termed a ‘second compartment’, hence herein the term ‘partition’ and ‘compartment’ may be used interchangeably. The same applies for ‘compartmentalization’ and ‘partitioning’.

‘Emulsion coupling’ or ‘coupling’ or ‘protein coupling’ or ‘PICO’ is a digital assay concept based on the detection of double-labelled (ternary), individual molecular complexes (e.g. couplexes) in emulsion, which are identified, for example, by dPCR or next-generation sequencing (NGS).

Preferably, targets can be concurrently assessed with other targets having different nature not suitable to be assayed by using unique nucleotide associated binding agents, especially RNA and DNA targets.

In these cases other methods can be advantageously combined with the invention, especially, methods detecting RNA expression of single-cells.

Preferably the target comprises a protein. More preferably the target is part of a protein sample. Optionally the target may be cross-linked to other targets within a plurality of targets e.g. a protein sample. For example, a protein within a sample may be cross-linked to one or more other proteins within the sample and forming the ternary or a higher order complex target as a cross-linked new entity.

The target may be associated with a unique nucleotide sequence. ‘Associated’ means that the presence of the target within the binding agent/target complex can be detected by the presence of the nucleic acid sequence within the linked sequence generated by the method. The nucleotide sequence may be attached as a label to the target, or be present within the target e.g., nucleic acid within a phage or the cross-linking process is used to associate a nucleotide sequence, e.g. DNA bound proteins are DNA, for example DNA is cross-linked to a histone protein. Alternatively, the nucleotide sequence may be part of an aptamer known to bind to the target. The binding agent/target complexes can be contacted with the aptamers to enable the target present to be identified through linking of the unique nucleotide sequences, including the aptamer. However, many alternative ways to associate labels are known in the art.

The present invention can be applied to any single-cell derived protein sample. Single-cells can be derived from any biological specimen including, but not limited to tissues, cytological specimens, body fluids, cell cultures or any other single-cell containing material. Body fluid samples include blood, saliva, urine, cerebrospinal fluid, and a buffy coat. Preparation of single-cell from specimens can be performed using standard methods known in the art (Hu, P., Zhang, W., Xin, H., & Deng, G. (2016). Single cell isolation and analysis. Frontiers in Cell and Developmental Biology, 4(OCT), 116. https://doi.org/10.3389/FCELL.2016.00116/BIBTEX). Among them, fluorescence activated cell sorting (FACS), magnetic-activated cell sorting (MACS), laser capture microdissection (LCM), manual cell picking/micromanipulation and different microfluidic platforms are more frequently applied. Recently, on-demand optically intercepted and controlled single-cell printing (A. Gross, J. Schöndube, S. Niekrawitz, W. Streule, L. Riegger, R. Zengerle, P. Koltay, Single-Cell Printer: Automated, On Demand, and Label J. Lab. Autom. 18 (2013) 504-518. Free, https://doi.org/10.1177/2211068213497204.).

As used herein, the term single-cell refers to one cell. Single cells useful in the methods described herein can be obtained from a tissue of interest, or from a biopsy, blood sample, or cell culture. Additionally, cells from specific organs, tissues, diseased tissue, or the like can be obtained and used in the methods described herein. Furthermore, in general, cells from any population can be used in the methods, such as a population of prokaryotic or eukaryotic single celled organisms including bacteria, fungi or yeast. Hence, preferably herein a sample is selected from the group comprising a tissue sample, a biopsy, a liquid biopsy, a blood sample, a plasma sample, a urine sample, a liquor sample, an environmental sample, a cell culture-derived sample and a sample derived from a microbiological culture.

In some embodiments of the method described herein, obtaining a sample can include the step of first obtaining single cells and then lysing the cells.

A single cell suspension can be obtained using standard methods known in the art including, for example, enzymatically using enzymes for tissue samples or releasing mechanically adherent cells in culture. Single cells can be placed in any suitable reaction vessel in which single cells can be treated individually. For example, a 96-well plate, such that each single cell is placed in a single well, this also can be termed compartmentalization. Individual cells can, for example, be individually selected based on features detectable by different analytical methods, based on measurable parameters such as location, morphology, or gene expression.

Methods of lysis of the cells are well known in the art. Lysis can be achieved by, for example, preferably by using detergents or other chemical methods, or by a combination of these.

The single-cell specimen can be chemically treated before the second compartmentalization, e.g. different be fixative chemicals or cross-linking agents can used (e.g. BS3-(bis(sulfosuccinimidyl) suberate) or formaldehyde and glutaraldehyde create bonds between lysine residues resulting in cross-linked proteins. Formaldehyde and glutaraldehyde is usually used at concentrations of 0.5-4% in PBS depending on the sample. The single-cell sample can be cross-linked or not cross-linked. Depending on the experimental objective and the type of target under investigation, single-cell targets can be analyzed either in their denatured or non-denatured form and/or cross-linked or not cross-linked form. The single-cell protein sample can be analyzed in a plurality of conditions to collect information about the quantitative characteristics of the plurality of targets. For example, the concentration or amount of the binding agent can be varied to determine dissociation constants and other kinetic parameters.

A separate permeabilization step also can be applied on single-cell to access intracellular targets. This allows binding agents to access intracellular structures while leaving the morphology of the cells intact. Triton, digitonin and saponin are examples of permeabilization reagents which act by disrupting the cellular membrane. The level of permeabilization is important as epitopes access may require different levels of permeabilization (e.g., cytoplasmic vs nuclear epitopes). Such techniques are commonly used in fluorescence activated cell sorting studies and well known in the art. There are also many commercial kits available today that provide the reagents to carry out both fixation and permeabilization. The invention however preferably does not need to remove unbound antibodies, as the preferred technique described in WO 2016/083793 A1 is a homogeneous assay, meaning it does not necessitate washing steps.

In embodiments the present invention relates to a method for conducting a homogeneous assay using said binding agents within a first partition exposed to lysed cellular material. In some embodiments wherein step d. (binding reaction) is performed after steps e. and f. (compartmentalization and lysis), an efficient and reliable analysis by additional binding of the binding agents under lysis conditions is enabled. This preferably prevents the interference of binding from cellular components. By employing such approach, the assay preferably achieves enhanced sensitivity and accuracy in detecting and quantifying analytes present in the lysed cellular material.

The single-cells can be preselected. For example, the single-cells can be an enrichment of specific proteins present e.g., proteins from a specific cellular location, from a specific cell type, of a similar size or electrostatic charge, proteins with similar binding properties, similar sequence characteristics, or similar functions e.g. enzymes (Current Protocols in Molecular Biology (2006) 20.0.1-20.0.6 CHAPTER 20 Analysis of Protein Interactions.). Preferably the specific proteins comprise phosphoproteins, membrane proteins or naturally, post-translational, artificially modified proteins.

The single-cells may present a protein display, extracellularly or intracellularly. Examples are well known in the art and described in [Galan, A., Comor, L., Horvatić, A., Kuleš, J., Guillemin, N., Mrljak, V., & Bhide, M. (2016). Library-based display technologies: where do we stand? Molecular BioSystems, 12(8), 2342-2358. https://doi.org/10.1039/C6MB00219F].

Targets can comprise many epitopes and can bind a plurality of binding agents. In one preferred embodiment all bound binding agents are pairwise detected by using a method of WO 2016/083793 A1 or WO2020260277A1.

The unit of detection is defined as the complex of the target and two bound binding agents and called couplex. If more than two binding agents are bound, all unique combinatorial combinations of binding agent pairs are considered as distinct couplexes.

A ‘label’ or a unique label herein comprises or consists of unique DNA, RNA and/or protein sequence. Any appropriate label known in the art may be used herein as label of a binding agent. Labels can be attached, linked or bound to primers, probes, molecules or binding agents covalently or non-covalently. In some embodiments a label may also comprise or consist of a fluorophore and/or a quencher.

For a next generation sequencing detection in embodiments the binding agents may preferably be labelled by unique PCR-amplifiable nucleic acid, preferably DNA labels, comprising a specific label for the type of binding gent (e.g. antibody) and preferably also an individual label for each binding agent molecule, i.e. a unique molecular barcode or unique molecular identifier-UMI (see Parekh et al., 2017). The labeled binding agents can in preferred embodiments be added to the samples or dilutions thereof.

For embodiments comprising a PCR, qPCR or dPCR detection, PCR reagents may be added before an emulsification of the samples or individually to every emulsion droplet (partition), e.g., by droplet fusion or microinjection. PCR or dPCR may be carried out using a standard dPCR protocol known in the art. The evaluation of the reaction may be based on the partitioning of the labels in a dPCR reaction using fluorescently tagged PCR products (e.g., using FAM- or VIC-labelled real-time PCR probes). According to the dPCR, standard evaluation the cluster of droplets may be determined according to the fluorescent signals of the droplets. Herein, the number of labelled binding agents (e.g., antibodies) is determined in each reaction (counting all label-positive droplets for a given label, and using the same definition of cluster of droplets for all reactions). Additionally, the number of the double-colored (having two different binding agent labels, e.g., of different types of binding agents, or of binding agents for different target analytes) is also determined. Without ternary complexes (couplexes), the partitioning of the labelled antibodies follows Poisson distribution, and results in a calculable number of double-colored droplets (having two binding components in one compartment based upon pure chance, but without binding to a present target). In the case of ternary complexes present in the reaction, the number of the detected e.g., double-colored, droplets (having additional ternary complexes) is larger than would be expected by Poisson distribution. On the basis of this measurements the number of couplexes can be calculated (see e.g., EP 20 3224360 or Karakus et al., 2019 for further reference). This results in an absolute (the count of molecules) quantitation of the ternary analyte complexes (couplexes).

In embodiments wherein multiple target analytes should be detected, a library encompassing binding agents that are respectively specific for the different analytes can be provided, wherein each binding agent comprises a label that is not only unique for the type of binding agent, but also for the specific analyte to be detected. For a low number of different target analytes to be detected said uniqueness can be encoded by different fluorescent labels with different colors. For larger numbers of different target analytes, the readout/analysis may be carried out by nucleic acid sequencing, such as NGS, in which case the labels should be nucleic acid sequences, that comprise a sequence that is unique for each target analyte and also for each type of binding agent. In embodiments droplet-specific or molecule-specific barcodes are added additionally to the label of each binding agent, either during the initial binding agent labelling reaction or at a later time point to each emulsion droplet (partition).

Per labeled molecules one or more labels may be used. Preferably, all labels are molecularly unique as described in WO2012042374A2 but read out using WO2020260277A1. Preferably, one labeled molecule has more labels and all of them are molecularly unique.

The unique labels can also be identity labels i.e., the associated unique sequences used in the binding agent and/or target to distinctly identify all members of binding agents and targets of the invention, which identity is not molecularly distinct identity of a label. Preferably, labels are both identity labels and molecular unique labels.

The unique labels may in embodiments be different in their biological background, and so the amplification and physical linking processes based on biologically specific different primer pairs, e.g., one or two or more primer pairs amplifies target sequences. Linking of the different labels makes it possible to link binding agent specific information to target information.

Performing the nucleic acid amplification for each compartment or partition to produce linked nucleic acid barcodes may be achieved by highly diluting the sample before the nucleic acid amplification, e.g. with a dilution factor of more than 20,000, preferably more than 50,000, more preferably more than 100,000. In preferred embodiments, the nucleic acid amplification is PCR. In other preferred embodiments the nucleic acid amplification is any suitable method known in the art. PCR reagents may be added to achieve single-complex separation and nucleic acid amplification per emulsion droplet or partition. To this end digital PCR (dPCR) standard protocols may be particularly suited. WO 2016/083793 A1 describes a method using linkage PCR concurrently, or only to generate linkage information. Afterwards the compartments, e.g. emulsion droplets, may be recombined in a common pool and a parallel nucleic acid sequencing technique can be used to assess antibody specific dimerized labels, e.g. UMIs. Herein, the number of labelled binding agents, e.g. antibodies, can be determined in each reaction by counting all unique labels (e.g. UMIs) for a given binding agent, e.g. antibody. The couplexes can be counted on the basis of their dimerized double (UMI-) labeled PCR products comprising two different binding-agent specific labels. Without the formation of ternary complexes (couplexes), the partitioning of the labelled binding agents follows Poisson distribution, and results in a calculable number of couplexes droplets. In the case of couplexes present in the reaction (target analytes present in the sample), the number of the detected couplexes is larger than would be expected by pure Poisson distribution.

Methods of attaching nucleotides to binding components such as antibody or compounds are known in the art. Alternatively, if the binding component library is a phage display library the unique nucleotide sequence can be the sequence that encodes one or more CDR regions or the displayed binding domain. For example, a display library can be generated by inserting sequences encoding the amino acid sequence to be displayed into a phage at a known location. Universal primers that will amplify the inserted sequences can then be used and thus identify the binding sequence.

Alternatively, in embodiments where the binding agent is an aptamer and the aptamer itself can be the unique nucleotide sequence. The nucleotide sequence may be an oligonucleotide and may comprise RNA or DNA, single or double stranded. Nucleotides used to label the binding agent or target can preferably be 5-150 bases in length, for example, 10-40, or 40-80 bases in length. The nucleotides that form the nucleic acid can be chemically modified to increase the stability of the molecule, to improve its bioavailability or to confer additional activity to it. For example, the pyrimidine bases may be modified at the 6 or 8 positions, and purine bases at the 5′-position with CH3 or halogens such as I, Br or Cl. Modifications or pyrimidines bases also include 2 —NH3, 0 —CH3, N—CH3 and N2-CH3. Modifications at the 2′-position are sugar modifications and include typically a NH2, F or OCH3 group. Modifications can also include 3′ and 5′ modifications such as capping. Alternatively, modified nucleotides, such as morpholino nucleotides, locked nucleic acids (LNA) and peptide nucleic acids (PNA) can be used. Morpholino oligonucleotides are assembled from different morpholino subunits, each of which contains one of the four genetic bases (adenine, cytosine, guanine, and thymine) linked to a 6-membered morpholine ring. The subunits
are joined by nonionic phosphorodiamidate inter-subunit linkages to give a morpholino oligonucleotide. LNA monomers are characterized in that the furanose ring conformation is restricted by a methylene linker that connects the 2′-0 position to the 4′-C position. PNA is an analogue of DNA in which the backbone is a pseudopeptide rather than a sugar.

### DESCRIPTION OF THE FIGURES

FIG. 1: dPCR readout of single cell with appropriate controls. NTC—no template control, containing either labeled antibodies or HER2 protein; ABC—containing antibodies (labeled trastuzumab, pertuzumab) at the same concentration as the samples, but not HER2 protein; BT474sc—single cells PICO reactions; rHER2 direct—recombinant HER2 PICO reactions.

FIG. 2: Graphical depiction (log-log) of simulated isomolar titration data (see table 1). X-axis scales the log of target molar concentration, Y-axis is the log of couplex molar concentration both at binding conditions of the bi-component method. The simulated preset concentration of target was 1.0E−11 M (isoCC), and the preset Kds are 5.0E−11 (Kdx1) and 1.0E−10 (Kdx2), the simulation is carried out under the data has a value of 1 of labelled fraction, a value of 1 of active fraction and no label error and free label, respectively. The antibody concentration is indicated at the curves and were set to 4.0E−11, 1.0E−11, 4.0E−10, 1.1E−9 and 5.0E−9. The concentration of couplexes are derived from solving equation described in WO2020260277A1. The recursively minimized standard deviation of antigen concentration was 0.18% (STDp) or 1.81E−14 (STD) by setting Kds to the preset values and isoCC was also reproduced.

FIG. 3: Graphical depiction (log-log) of simulated isomolar titration data (see table 1 and 3) at the value of 0.5 of active fraction other conditions are like in FIG. 2. X-axis scales the log of target molar concentration, Y-axis is the log of couplex molar concentration both at binding conditions of the bi-component method. The preset concentration of target was 1.0E−11 M (isoCC). FIG. 3A shows data from Table 3. Apparent Kds 1.0E−10 (Kdx1) and 1.0E−10 (Kdx2) are, respectively. The recursively minimized standard deviation of antigen concentration was 0.12% (STDp) or 1.16E−14 (STD). It is notably different from the preset values of Kds (see FIG. 2.) and represents new apparent Kds, noteworthy that the preset isoCC value is recovered. FIG. 3B shows data from Table 2., the recursively minimized standard deviation of antigen concentration was 2.28% (STDp) or 2.33E−13 (STD), where the (apparent) Kds are 5.0E−11 (Kdx1) and 1.0E−10 (Kdx2) respectively. These values are identical to the values of FIG. 2. (A) describes the case when the active fraction is unknown, and the active concentration of antibody is falsely determined on the basis of labeled antibodies (the real concentration is reduced as the inactive antibodies are non-reactive). It is shown that after recursive minimization of the standard deviation of antigen concentration the isoCC is identical to the original isoCC, but the apparent Kd are not. (B) the control simulation. The antibody concentration is adjusted according to the active fraction, the original Kds are reproduced along with the original isoCC.

FIG. 4: Graphical depiction (log-log) of simulated isomolar titration data (see table 4) at value of 0.5 of labeled fraction. X-axis scales the log of target molar concentration, Y-axis is the log of couplex molar concentration both at binding conditions of the bi-component method. The preset concentration of the target was 1.0E−11 M (isoCC). FIG. 4A depicts the apparent antibody and couplex concentrations were changed, according to simulating value of 0.5 of labeled fraction, and indicated antibody concentration were 2.0E−11, 5.0E−11, 2.0E−10, 5.5E−10 and 2.50E−9 (half amount as only the labeled fraction is detected). Noteworthy that in case of no recursive minimization where the Kds 5.0E−11 (Kdx1) and 1.0E−10 (Kdx2) are unchanged, respectively, the standard deviation of antigen concentration was 17.11% (STDp) or 1.04E−12 (STD). This is due to the not perfect fit of the simulated target concentrations. FIG. 4B shows, that after recursive minimization, the standard deviation of antigen concentration was 0.37% (STDp) or 1.84E−14 (STD), the apparent Kds 2.60E−11 (Kdx1) and 1.0E−10 (Kdx2) are, reproducing the original values, respectively, however the concentration of antigen (isoCC) was 5.0E−12. As the 0.5 of labeled fraction, the antigen concentration needs to be adjusted resulting in 1.0E−11 M, which identical with the original antigen concentration.

FIG. 5: The figure shows the Agilent Protein 230 Chip on the 2100 Bioanalyzer electro-phoretogram of labeled antibody of anti-GAPDH. The labeled fraction was determined according to Example 5 and was 97.6%.

FIG. 6: The figure shows the graphical depiction (log-log) of the determination of the apparent dissociation constants of trastuzumab and pertuzumab. X-axis scales the log of target molar concentration, Y-axis is the log of couplex molar concentration both at binding conditions of the bi-component method. Samples have isomolar concentrations, however the exact concentration of HER2 is unknown (the sample was obtained from lysed HER2 cells), and the concentration of the antibodies varied (5.5E−9, 1.50E−9, 4.0E−10, 1.2E−10, 3.5E−11 and 1.0E−11 for antibodies). Red lines are calibration curves of HER2 specific bi-component assay similar to the method described in WO2020260277A1 assay at these antibody concentrations and apparent Kds. The latter is determined recursively minimizing the SD of the recalculated HER2 concentrations for all measurement under the assumption of that all samples contains the same concentration of HER2. The molar HER2 concentration is designated as MeanHER2 (identical to isoCC in other depictions), STD and pSTD are the standard deviation of measurement, and its percentage of mean, respectively. The dotted lines are the means of HER2 measurements at different antibody concentrations. Finally, the dissociation of antibodies (Kd1TTZ and Kd2PTZ) are recursively determined to be 2.9E−11 and 9.3E−11, respectively.

### EXAMPLES

### Example 1

This example provides an example of the first compartmentalization of the binding reaction between the cells and antibodies, which can be preferably carried out at high cell diffusion-limited concentration. The concentration of target proteins (target analytes) of the binding reaction is preferably defined by the volume of the cell suspension and the total target protein content in that volume. Under suitable conditions and assumptions, the concentration of the target protein/analyte can be treated as it is in solution, as shown in the present Example. Hence, their concentration per cell can, in such embodiments, be assumed from their concentration calculated for the entire sample volume and the numbers of cells in said sample volume. Separating each single-cell and having a suitable sensitive method, the number of targets per cell can be determined from the extrapolated concentration of the target in the suspension volume multiplied by the number of cells in suspension. Preferably, this method comprises a second compartmentalization which can be used to count single molecules including both couplexes and binding agents. Preferably, the cells are lysed before the second compartmentalization. Preferably, the extrapolated concentration of the target in the suspension volume is determined using the binding characteristics and finally results in the determination of the absolute amount of the target analyte per single-cell, as described in the present Example. The present Example further evidences that the present method, due to the double compartmentalization described herein, surprisingly facilitates the detection of target analyte concentrations below 1000, or 100 copies or 10 copies per cell.

Methods Employed in this Example

MCF7 (ATCCR HTB-22) and BT-474 (ATCC® HTB-20) cells were obtained from the BIOSS Centre for Biological Signaling Studies (Freiburg, Germany). MCF7 cells were cultured in DMEM, GlutaMAX Supplement (31966021, Gibco) and BT-474 cells were cultured in DMEM/F12, GlutaMAX Supplement (31331028, Gibco) in Nunc EasYFlask Cell Culture Flasks (156340, Thermo Scientific™). Both media were supplemented with 10% FBS (10270106, Gibco) and 1% Pen/Strep (15140122, Gibco). Cells were cultured until ~90% confluency in a cell culture incubator (Heracell™ 150i CO2 Incubator, 50116048, Thermo Scientific™) under a 5% CO₂ atmosphere at 37° C. Cells were harvested scraping. The cells were washed twice with DPBS (14040133, Gibco) and counted (Countess® ∥ Automated Cell Counter, Invitrogen™) including live/dead staining with trypan blue (T10282, Invitrogen™).

The Immediate Drop-on-demand Technology (I.DOT One; Dispendix, Stuttgart, Germany) [50,51] with I.DOT PURE plates 90 μm orifice (Dispendix, Stuttgart, Germany) was used to dispense 0.5 μl LBTW into a 384-well V-bottom plate (0030623304, Eppendorf). Prior to dispensation, the I.DOT was calibrated for the applied liquids to ensure reliable dispensation. The liquid class ‘H2O’ was used to dispense the diluted crude cell lysate (‘cl’) or DPBS.

The binding agents were in the present example antibodies that were conjugated with DNA labels using a proprietary procedure of Actome GmbH, Germany. The achieved labeling efficiencies were between 80-100% and were confirmed by SDS-PAGE electrophoresis. The antibody preparations have 3% free labels, 25% labeling errors (see Example 5). Trastuzumab (TTZ) and pertuzumab (PTZ) are recombinant humanized monoclonal antibodies, both targeting extracellular regions of the HER2 tyrosine kinase receptor and having no overlapping epitopes. The conjugated antibodies have two different labels-trastuzumab BL, and pertuzumab P8 labels. Apparent dissociation constants of antibodies were determined according to Example 6 and were TTZ=0.49E−10 M and PTZ=0.93E−10, respectively.

The bi-component assay used were similar to that described in WO 2016/083793 A1, is included herein as a reference. Briefly, MCF7 and BT-474 cell concentrations were adjusted to 1×10⁷ cells/ml and 100 μL of cell suspension were overnight incubated with the conjugated antibodies (ABX-mixture of antibodies) or alternatively, the cell suspension was lysed using proprietary lysis buffer supplied by Actome GmbH, Germany and the lysate was incubated with the antibodies (adding ABX) overnight.

In case of non-lysed cells, the cell dispensing procedure was performed as described earlier (Groß, C. Jeney, D. Halm, G. Finkenzeller, G. B. Stark, R. Zengerle, P. Koltay, S. Zimmermann). Characterization of CRISPR/Cas9 RANKL knockout mesenchymal stem cell clones based on single-cell printing technology and emulsion coupling assay as a low-cellularity workflow for single-cell cloning, BioRxiv. (2020) 1-19. https://doi.org/10.1101/2020.08.17.253559.). Briefly, using the F.SIGHT™ single-cell dispenser (CYTENA GmbH, Freiburg, Germany), an improved version of the single-cell printer (SCP) (A. Gross, J. Schöndube, S. Niekrawitz, W. Streule, L. Riegger, R. Zengerle, P. Koltay, Single-Cell Printer: Automated, On Demand, and Label Free, J. Lab. Autom. 18 (2013) 504-518. https://doi.org/10.1177/2211068213497204.). Both MCF7 and BT-474 cell concentrations were adjusted to 1×10e6 cells/ml and loaded into a dispensing cartridge (CYTENA GmbH, Freiburg, Germany). The settings for MCF7 cells were 10 to 25 μm of cell size (BT-474:10 to 30 μm) and 0.5 to 1 of roundness (same for BT-474), respectively. The F.SIGHT can reliably dispense single cells in minimal liquid volumes. The single-cell dispensation efficiency (successful single-cell isolation events divided by targeted events) is usually around 90%, and is additionally controlled by cell images unambiguously assigned to each dispensation event. Thus, other than single-cell dispensation events like doublets or empty droplets can be excluded.

QIAGEN's QIAcuity Probe Mastermix (41 μL) was added, and the samples (0.5 μL for both lysed bulk and single-cell samples) were loaded onto a QIAcuity Nanoplate 26 k 24-well plate. Digital PCR was performed using a QIAGEN QIAcuity Digital PCR System with cycling parameters according to the PCR conditions of hot-start 95° C. for 2 min, cycling 40 times, denaturing 95° C. for 15 sec, annealing 58° C. for 30 sec. Imaging conditions P8 label-FAM green channel, 500 ms integration time, gain 6 and BL label-HEX yellow channel, 400 ms integration time, gain 6.

The cells were treated according to the following steps: (a) single-cells were incubated at 1×10e6 cells/50 μl with labeled PTZ and TTZ respectively. (b) After overnight incubation cells were printed into 0.5 μL (I-DOT dispended) lysis buffer. (c) QIAcuity Probe Mastermix (41 μL) was added, and the samples (0.5 μL for both lysed bulk and single-cell samples) were loaded onto a QIAcuity. A negative control containing only labeled trastuzumab and pertuzumab antibodies was also set up (antibody binding control) derived form the cell free buffer or from the ABX antibody mixture.

The calibration curve referred to herein is derived similar as described in WO 2020/260277 A1, however applying the herein described binding characteristics.

In case of bulk lysate, the calibration curve shows the molar concentration of the target analyte (in the present example HER2 protein). The number of couplexes in the dPCR reaction of dispensed single-cell equivalent amounts of lysate was read out directly, multiplied by the dilution factor of the binding reaction to get the molar concentration of couplexes in the binding reaction, wherein the dilution factor of the binding reaction is determined by the fraction of the volume of the binding reaction being measured in the dPCR reaction. The molar concentration of antibodies was also similarly determined, the row counts were obtained from the direct reading of the dPCR reaction and similarly applying the dilution factor of the binding reaction. Apparent dissociation constants of antibodies were determined previously. Using the binding characteristics-compensated values of the absolute concentration of analytes in the bulk binding reaction was determined.

In case of single-cells, the calibration curve shows the number of target analytes (HER2 protein) determined per single-cells. The molar concentration of antibodies and couplexes in the binding reaction was determined similarly applying the dilution factor of the binding reaction. However, the dilution factor was calculated differently, as single-cells represent the single-cell fraction of the target protein (analyte), the number of couplexes of the measured single-cells were multiplied by the number of cells in the suspension volume, such that the number of cells in the suspension volume was treated as the dilution factor of the binding reaction. Apparent dissociation constants of antibodies were determined previously. Using binding characteristics-compensated molar inputs and apparent dissociation constants of the antibodies the absolute number of target analytes in the suspension (sample) is determined. Briefly, considering all cells in the suspension the molar concentration of couplexes and binding agents was calculated, in the suspension assuming solution based conditions during the calculations. The chemical calculations are carried out, similar as described in WO 2020/260277 A1, to determine the absolute molar concentrations. Given the molar concentrations of the couplexes and binding agents are derived from each individual single-cells this a mathematical compensation of unbound, non-couplex forming targets resulting in absolute concentrations. Gaining the absolute (compensated) molar concentration of the target and considering the number of cells in the suspension, the absolute count of target analytes per single cell was determined. The number of target proteins per single-cells was derived from the absolute concentration of targets by dividing by the number of cells in the suspension.

For the validity of this method it was assumed that the target concentration on single-cells is lower than the antibody concentration in the cell suspension not to reduce significantly the free antibody concentrations. However, at physiological concentration of proteins in the cells normally the assumption holds and also for those skilled in the art recursive calculations are known to compensate.

Noteworthy, that the bulk lysate of cells contains more detectable HER2 molecule than single-cells, as the single-cell detection, in the absence of permeabilization, is restricted to the cell surface only.

In the case of MCF7 cells, the average number of HER2 proteins per single cell is 4500±1,060 molecules, whereas the expected number under bulk conditions is 10,150±1,260 molecules. Similarly, BT474 cells exhibit approximately 208,000±1,260 HER2 proteins per single cell compared to the bulk condition result of 2.3E+6±8.E+5 molecules. These disparities in HER2 protein quantities are attributed to the inherent distinctions between surface-accessible and bulk-accessible protein pools. The method disclosed herein provides a reliable means to quantify HER2 proteins in single cells and compare them to bulk conditions, thereby facilitating a comprehensive understanding of the protein distribution at both cellular and population levels.

### Example 2

Plate-Based ‘scPICO’ Method According to the Invention

### Reagents and Buffers

The reagents and buffers were prepared as listed below on the day of the experiment.

Additive C (5× stock)
500 μl PBS

### BSA (5× Stock)

20 mg BSA
400 μl PBS

### EDTA-free Protease Inhibitor Cocktail (PIC), (25× Stock)

1 tablet of complete Protease™ Inhibitor Cocktail
PBS to 2 ml

### Cell Lysis Buffer (LBT), (2× Stock)

200 μl Additive T (10×)
400 μl dissolved Additive C (5×)
80 μl PIC (25×)
200 μl Additive L
120 μl PBS

### Cell Lysis Buffer (LBTW), (1×)

300 μl LBT Buffer (2× stock)
300 μl PBS
Antibody Binding Control (ABC) Buffer, (1×)
250 μl LBT Buffer (2× stock)
100 μl BSA (5×)
150 μl PBS

### Oligonucleotides

| SEQ ID | Oligo- | | |
|---|---|---|---|
| NO. | nucleotide | Type | Sequence |
| 1 | GV_M13VIIIF1 | Primer | GGTGACGATCCCGCAAAA |
| 2 | IM_M13VIIIR1D1 | Primer | TGCGCATCCATTGTAGAGGTGGTCGCTGAGGCTTGCA |
| 3 | IM_M13VIIIR1D2 | Primer | ACCTCTACAATGGATGCGCAGGTCGCTGAGGCTTGCA |
| 4 | IM_M13VIII50U1F1 | Primer | AGTAACAACCGATCCCGCAAAA |
| 5 | P8_MGB_FAM | Probe | CGGCCTTTAACTCC |
| 6 | NOS6_MGB_ | Probe | ACCTACCGGCCTCC |
| | ATTO550 | | |
| 7 | AC_NOS6_B_ | Label | TTTTTGGTGACGATCCCGCAAAATNNNNNNNNNNCACCTACC |
| | D0_L1 | | GGCCTCCTGCAAGCCTCAGCGACC |
| 8 | AC_NOS6_0_ | Label | TTTTTGGTGACGATCCCGCAAAATCACCTACCGGCCTCCTGC |
| | D0_L1 | | AAGCCTCAGCGACC |
| 9 | AC_P8_B_ | Label | TTTTTGGTGACGATCCCGCAAAAGNNNNNNNNNNCGGCCTTT |
| | D0_L1 | | AACTCCCTGCAAGCCTCAGCGACC |

### Antibody Mix

For the detection of HER2 in single BT474 cells two anti-HER2 humanized monoclonal antibodies, pertuzumab (PTZ) and trastuzumab (TTZ), were used. Both antibodies were covalently modified with DNA oligonucleotide labels using the PICOglue technology. The labels contain a 10 base pair random nucleotide sequence (UMI) and a TaqMan hydrolysis probe annealing sites for binding of P8 and NOS-6, respectively (PTZ-P8 and TTZ-NOS6) (see below). The antibody mix was prepared by dilution of the labeled antibody stocks in LBTW to a molar concentration of 5.00×10⁻¹⁰ M of each antibody. The antibody mix was subsequently diluted 1:2 in LBTW and stained with fluorescein to a final concentration of 1 μM. The applied concentration was intended to provide saturating conditions.

### Cell Culture

Breast ductal carcinoma BT474 cells were grown in DMEM F12 media supplemented with GlutaMax+10% FCS+1% P/S in T175 flasks and split when 80-90% confluent 1:2 to 1:3 every 5-7 days. Cells were harvested after washing with 10 ml of PBS by incubation with 5 ml of Versene non-enzymatic cell dissociation reagent (Thermo) for 3-5 minutes at room temperature. When cells started to detach the reaction was stopped by dilution 1:2 with PBS before the cell suspension was centrifuged at 400 g for 5 min. Then cells were resuspended in 40 ml of fresh media per T175 flask and seeded.

For the single cell analysis according to the invention (“single-cell PICO approach”) a T175 flask of BT474 cell culture was harvested as described above and resuspended in 1000 μl of PBS and subsequently filtered through a 30 μm cell strainer. Cells were counted, washed again with 1 ml PBS by centrifugation at 400 g for 2 min and resuspended in PBS stained with 100 μM Fluorescein to a density of approximately 0.5×10⁻⁶ cells/ml. Cells were stored on ice until use.

### Preparation of 384 Well Plate and Single-Cell Isolation

The wells of a 384 well Eppendorf Microplate 384/V-PP were prefilled with 4 μl Vapor-Lock PCR overlay (Qiagen) and briefly spun down at 1000 g. To each well 12 nl of antibody mix (1:2 diluted in LBTW; 1 μM fluorescein) were dispensed using the I.DOT liquid handler (Dispendix). The plate was then centrifuged for 2 min at 2000 g to ensure droplet positioning at the center of the well under the Vapor-Lock overlay. Successful droplet dispensing and positioning was controlled microscopically with a Dino-Light fluorescent microscope (AM4115T-GFBW).

Single BT474 cells were isolated with the f.sight single-cell dispenser (Cytena) with automated offset correction (AOC) enabled. Single cells with a size of 15-25 μm and a roundness setting of 0.6-1.0 were selected and only reactions receiving one single cell were valid for downstream processing.

Merge of the antibody mix droplet and the single-cell containing droplet was considered successful when an increase of the fluorescent signal was observed compared to the reactions not receiving a single cell. The additional reactions not receiving a single cell also served as in-plate antibody binding controls (ip ABCs) (see below).

After single-cell isolation the plate was sealed with a PCR foil (4titude) and incubated for approximately 24 hours at 4° C., such that both cell lysis and the binding reaction between HER2 and the two antibodies (couplex-formation) could occur during the incubation.

coupling PCR on Stilla Naica

| | stock conc. | final conc. | |
|---|---|---|---|
| | (μM) | (nM) | volume |
| Total volume per reaction (μl) | | | 25 |
| Perfecta Multiplex qPCR Tough Mix | | | 5.00 |
| input (sample dilution) | | | 1.00 |
| Fluorescein | 10 | 100 | 0.25 |
| GV_M13VIIIF1 | 10 | 400 | 1.00 |
| IM_M13VIIIR1D1 | 1 | 20 | 0.50 |
| IM_M13VIIIR1D2 | 1 | 20 | 0.50 |
| IM_M13VIII50U1F1 | 10 | 200 | 0.50 |
| P8_MGB_FAM | 10 | 250 | 0.63 |
| NOS6_MGB_ATTO550 | 10 | 250 | 0.63 |
| Subtotal | | | 10.00 |
| Plus H₂O | | | 15.00 |

### Preparation of Control Binding Reactions and Sample Dilution

Before being used as an input for dPCR the 12 nl single-cell binding reactions were diluted in PBS as follows. To achieve a 1:5000 predilution, 60 μl of PBS were added directly to the single-cell reaction followed by repeated mixing by pipetting and centrifugation at 2000 g for 2 min. 5 μl of the pre-dilution was transferred to 42.5 μl of PBS (1:9.5) and then mixed by repeated pipetting. 1 μl and 1.32 μl of this dilution was used as input for the Stilla and QIAcuity dPCR mastermix, respectively. The input amount is correct for the partition size of 0.78 nl and 0.59 nl on the Stilla Sapphire Chip and the QIAcuity system, respectively, to achieve a lambda of 0.15.

This corresponds to a total dilution factor of approximately 1.19×10⁶ and 1.52×10⁶, respectively.

dPCR Master Mix

Standard PCR on QIAcuity

| | stock conc. | final conc. | |
|---|---|---|---|
| | (μM) | (nM) | volume |
| Total volume per reaction (μl) | | | 42 |
| QIAcuity Probe Mix | 4 | 1 | 10.50 |
| input (sample dilution) | | | 1.32 |
| primer mix 800 nM each (25 μM | 25 | 0.8 | 1.34 |
| stock) | | | |
| P8_MGB_FAM | 10 | 0.4 | 1.68 |
| NOS6_MGB_ATTO550 | 10 | 0.4 | 1.68 |
| Subtotal | | | 16.52 |
| Additional H₂O | | | 25.48 |

Partitioning and dPCR reactions were carried out on the Naica Geode system with Sapphire Chips (Stilla) for the coupling PCR and on the QIAcuity System (Qiagen) using the Nanoplate 24 26 k.

### Naica Sapphire System-dPCR Parameters:

1. Partition at 40° C. Sapphire V1
2. Temp 95° C. for 10 min
3. Start Cycle 40
   Temp 95° C. for 30 seconds
   Temp 60° C. for 15 seconds
4. Start Cycle 20
   Temp 95° C. for 30 seconds
   Temp 50° C. for 15 seconds
5. Close Cycle
6. Release P, Sapphire V1
   QIAcuity System—Nanoplate 24 26k—dPCR Parameters:
1. QIAcuity standard priming
2. Temp 95° C. for 2 min
3. Start Cycle 40
   Temp 95° C. for 30 seconds
   Temp 60° C. for 15 seconds
4. Start Cycle 20
   Temp 95° C. for 30 seconds
   Temp 50° C. for 15 seconds
5. Close Cycle
   dPCR Reactions

Emulsification, ddPCR, and readout were performed using the Stilla Naica System following standard dPCR protocol. As a negative control, the antibodies were mixed without antigen and processed in the same way as the samples (antibody-binding-control, ABC).

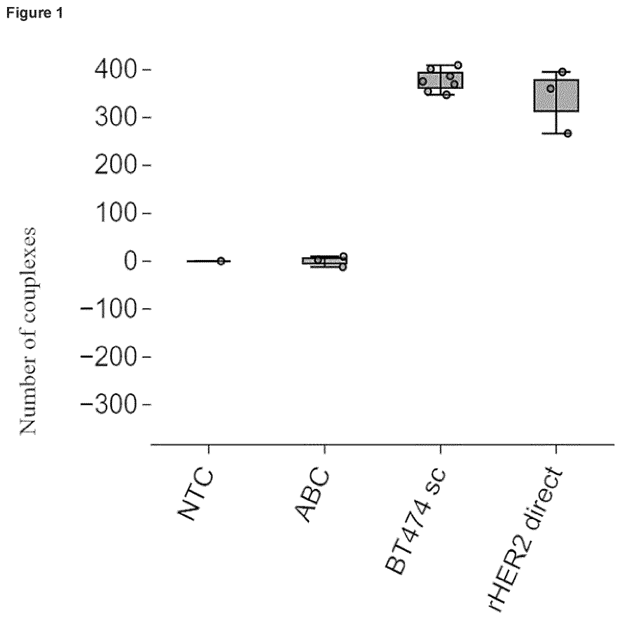

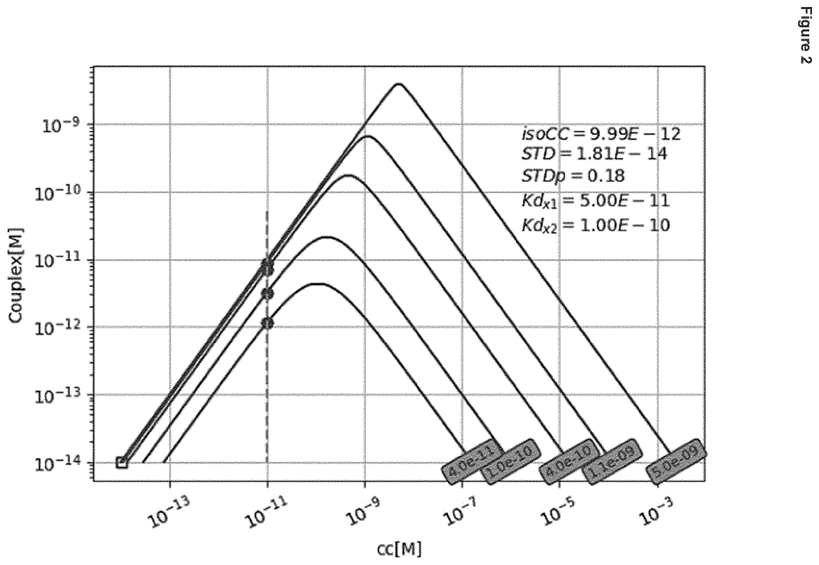

The amount of protein HER2 was measured by detecting the partitioning differences induced by concurrent binding of the antibodies to HER2. The measurements were normalized against the ABC control, which defines the zero level detection by the ABC signal. FIG. 1 shows the results of the dPCR of single cell analysis with appropriate controls.

NTC and ABC readings were zero indicating contamination free dPCR reactions and good balanced dPCR readout, BT474sc samples indicating around 987,000 (±7.6%) HER2 molecules detected per cells on average considering the number of detected couplexes using the applied dilution and other factors effecting the count of the couplexes (as described in detail in Karakus et al. 2019, EP 22161450.6, which are included herein by reference), while rHER2 sample indicates about 876,000 (±9.8%) HER2 molecules considering a similar procedure.

Droplet Recovery from Sapphire Chips and DNA Extraction

First the inlet and outlet of the sapphire chips were completely emptied with a pipette and the collected oil was transferred to a collection tube. Then an Eppendorf Combitip advanced (1.0 mL, yellow) was inserted firmly into the outlet. Then each inlet was filled up with 50 μl Novec 7500. Subsequently, the emulsion within the chip was recovered by slowly pulling the Combitip piston until the liquid level in the inlet decreased to the bottom. This process was repeated once more and collected liquid was transferred to a collection tube. Next, the tubes were centrifuged for 2 min and the oil phase was removed from the bottom of the tube using a pipette. Demulsification was carried out by the addition of 60 μl 20% PFO (v/v) in Novec 7500, vortexing for 10 seconds and centrifugation for 2 min at 1000 g. Separation of aqueous phase and oil phase was achieved by slowly turning the tubes upside down and collecting the aqueous phase from the tube bottom.

### NGS Library Preparation

For NGS library preparation the NEBNext Ultra II Library Prep was used according to the manufactures protocol with the following settings and modifications. All reaction volumes were reduced by 50% and as input approximately 5 ng of unpurified amplicon DNA was used. The NEB adapter was diluted 25-Fold (1:25) to 0.6 μM in 10 mM Tris-HCl, pH 8.0 with 10 mM NaCl and no size selection was performed before PCR. The library DNA was purified with 22.5 μl (0.9×) resuspended beads (NEB) according to protocol.

### NGS on the Illumina MiSeq Platform

The Library was pooled equimolar and NGS was performed using the MiSeq platform with the MiSeq Reagent Kit v2 with 300-cycles paired-end according to the manufacturer's protocol. After denaturation the library pool was diluted 5 PM and 10% PhiX control was spiked in.

NGS results were calculated according to the procedure described in (PCT/EP2020/067493), briefly the compartmentalized dPCR was carried out using molecularly unique labels (UMIs) and the dimers were deconvoluted to reconstruct the content of the droplets (in which the ddPCR was performed, preferably only comprising one couplex). Namely, the evaluation of the reaction may be based on the NGS reading of the binding agent, e.g. antibody, specific dimerized UMI labels generated according to the standard protocol of emulsion coupling. The number of labelled binding agents, e.g. antibodies, may be determined in each reaction by counting all unique UMI labels for a given binding agent, e.g. antibody (counting restricted to a given binding agent). Possible multiple labelling of the same binding agent, e.g. antibody, can be eliminated using their preferentially dimerized sequences (multiple labels per binding agent will always result in double UMI label dimers with a given antibody specific label context, as they co-localizing in the same droplet). Couplexes are counted based on their dimerized double UMI labeled PCR products (in the context of two different binding agent type-specific labels (e.g. antibody against a specific HER2 antigen)).

Once the droplet content was obtained a Poisson-based calculation (as described in EP 22161450.6) was carried out confirming the number of detected couplexes. Briefly, the distribution of binding agents during compartmentalization is governed by Poisson distribution, thus counting the occurrences of each binding agent by direct dPCR (or by the determination of their abundances of binding agents by NGS after droplet-based PCR amplification), if the number of droplets are known, thus the background detection of couplexes can be calculated. A multitude of specific binding events will be used to identify the exact target agents of the binding agents can be carried out in the same manner. E.g., in multi-protein complexes the information contained in the linked labels of binding agents is based on the co-localization of respective binding agents on said complex, and indicates direct interaction of detected target analytes with each other. As for each protein/protein complex, when the background detection is calculated any detected variation of protein/protein complexes is due to different interactions, which can be calculated by removing the calculated background detection of proteins/protein complexes (or Poisson-corrected subtraction as proteins/protein complexes bound to binding agents change the overall number of binding agents). The ABC compensated numbers are BT474sc samples indicating around 943,000 (+14.8%) HER2 molecules detected per cells.

### Example 3

The present example provides an example how the dissociation constants (Kd) of binding agents, or binding characteristics under various conditions of labeled ratio, label synthesis error, active fraction, and contamination with free labels, can be determined to be subsequently applied in the method according to the present invention in order to derive absolute amounts of the formed couplexes (or ternary complexes), facilitating a target standard free determination of dissociation constant of antibody in a complex chemical environment, also in a native environment.

TABLE 1
Simulated isomolar titration data. Column headers are:
‘name’—sample name, ‘couplex’—two antibodies -
target complexes, ‘ableft’—concentration of
antibody x1, ‘right’—concentration of antibody x2,
all concentrations are in M. ABX means ‘antibody
mix’ and they contain equimolar concentration
of the two antibodies indicated.

| name | couplex | ableft | abright |
|---|---|---|---|
| ABC | 7.06E−12 | 4E−10 | 4E−10 |
| ABC | 7.06E−12 | 4E−10 | 4E−10 |
| ABC | 7.06E−12 | 4E−10 | 4E−10 |
| ABX#1 | 9.71E−12 | 5E−09 | 5E−09 |
| ABX#1 | 9.71E−12 | 5E−09 | 5E−09 |
| ABX#1 | 9.71E−12 | 5E−09 | 5E−09 |
| ABX#1 | 9.71E−12 | 5E−09 | 5E−09 |
| ABX#2 | 8.76E−12 | 1.1E−09 | 1.1E−09 |
| ABX#2 | 8.76E−12 | 1.1E−09 | 1.1E−09 |
| ABX#2 | 8.76E−12 | 1.1E−09 | 1.1E−09 |
| ABX#2 | 8.76E−12 | 1.1E−09 | 1.1E−09 |
| ABX#3 | 7.06E−12 | 4E−10 | 4E−10 |
| ABX#3 | 7.06E−12 | 4E−10 | 4E−10 |
| ABX#3 | 7.06E−12 | 4E−10 | 4E−10 |
| ABX#3 | 7.06E−12 | 4E−10 | 4E−10 |
| ABX#4 | 3.18E−12 | 1E−10 | 1E−10 |
| ABX#4 | 3.18E−12 | 1E−10 | 1E−10 |
| ABX#4 | 3.18E−12 | 1E−10 | 1E−10 |
| ABX#4 | 3.18E−12 | 1E−10 | 1E−10 |
| ABX#5 | 1.13E−12 | 4E−11 | 4E−11 |
| ABX#5 | 1.13E−12 | 4E−11 | 4E−11 |
| ABX#5 | 1.13E−12 | 4E−11 | 4E−11 |
| ABX#5 | 1.13E−12 | 4E−11 | 4E−11 |
| NTC | 0 | 0 | 0 |

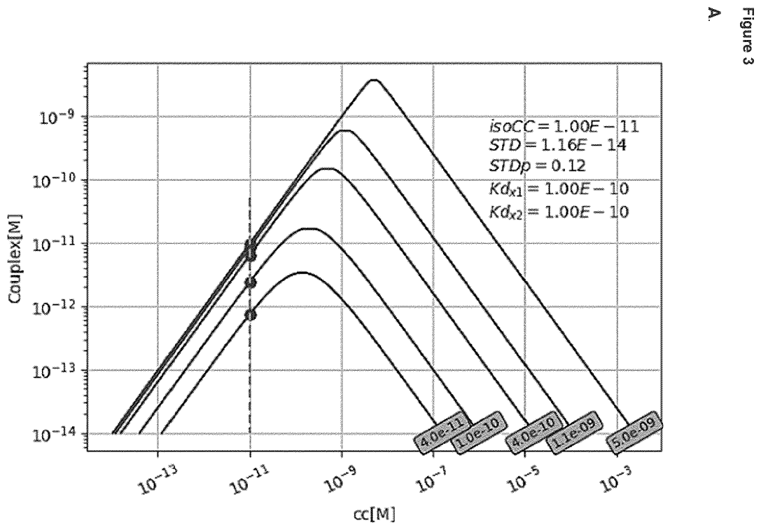

The FIG. 2 shows the graphical depiction of simulated isomolar titration data (see table 1). The preset concentration of target was 1.0E−11 M (isoCC), and the preset Kds are 5.0E−11 (Kdx1) and 1.0E−10 (Kdx2), the data has a value of 1 of labelled fraction, a value of 1 of active fraction and no label error and free label, respectively. The antibody concentration is indicated at the curves and were set to 4.0E−11, 1.0E−11, 4.0E−10, 1.1E−9 and 5.0E−9. The concentration of couplexes are derived from solving the equation described in WO2020260277A1. The recursively minimized standard deviation of antigen concentration was 0.18% (STDp) or 1.81E−14 (STD).

TABLE 2
Simulated isomolar titration data changing the active concentration
of antibody x1 to a value of 0.5 of active fraction x1 for determining
the new couplex concentrations using the preset Kd of antibodies.
Column headers are: ‘name’—sample name, ‘couplex’—couplexes,
‘ableft’—concentration of antibody x1, ‘right’—concentration
of antibody x2, all concentrations are in M. ABX means ‘antibody
mix’ and they contain the concentration of the two antibodies
indicated. The data shows if the ‘ableft’ has an amount of
the half of the original values the corresponding couplex values
are also lower (amount of couplex was determined by simulation).

| name | couplex | ableft | abright |
|---|---|---|---|
| ABC | 5.27E−12 | 2E−10 | 4E−10 |
| ABC | 5.27E−12 | 2E−10 | 4E−10 |
| ABC | 5.27E−12 | 2E−10 | 4E−10 |
| ABX#1 | 9.65E−12 | 2.5E−09 | 5E−09 |
| ABX#1 | 9.65E−12 | 2.5E−09 | 5E−09 |
| ABX#1 | 9.65E−12 | 2.5E−09 | 5E−09 |
| ABX#1 | 9.65E−12 | 2.5E−09 | 5E−09 |
| ABX#2 | 8.42E−12 | 5.5E−10 | 1.1E−09 |
| ABX#2 | 8.42E−12 | 5.5E−10 | 1.1E−09 |
| ABX#2 | 8.42E−12 | 5.5E−10 | 1.1E−09 |
| ABX#2 | 8.42E−12 | 5.5E−10 | 1.1E−09 |
| ABX#3 | 6.38E−12 | 2E−10 | 4E−10 |
| ABX#3 | 6.38E−12 | 2E−10 | 4E−10 |
| ABX#3 | 6.38E−12 | 2E−10 | 4E−10 |
| ABX#3 | 6.38E−12 | 2E−10 | 4E−10 |
| ABX#4 | 2.38E−12 | 5E−11 | 1E−10 |
| ABX#4 | 2.38E−12 | 5E−11 | 1E−10 |
| ABX#4 | 2.38E−12 | 5E−11 | 1E−10 |
| ABX#4 | 2.38E−12 | 5E−11 | 1E−10 |
| ABX#5 | 7.4E−13 | 2E−11 | 4E−11 |
| ABX#5 | 7.4E−13 | 2E−11 | 4E−11 |
| ABX#5 | 7.4E−13 | 2E−11 | 4F−11 |
| ABX#5 | 7.4E−13 | 2E−11 | 4E−11 |
| NTC | 0 | 0 | 0 |

TABLE 3
Simulated isomolar titration data using the couplex concentrations
at a value of 0.5 of active fraction of antibody x1 from Table
2 for determining the new apparent Kds of antibodies. The concentration
of couplexes is the same as in Table 2. representing a value of
0.5 active fraction, but the active fraction was considered to
be unknown and the ‘ableft’ was not compensated as in Table
2. Column headers are: ‘name’—sample name, ‘couplex’—two
antibodies - target complexes, ‘ableft’—concentration of
antibody x1, ‘right’—concentration of antibody x2, all
concentrations are in M. ABX means ‘antibody mix’ and they
contain the concentration of the two antibodies indicated. ‘ableft’
values are adjusted if the active fraction is 1, but the couplex
concentrations are according to the value of 0.5 of active fraction.

| name | couplex | ableft | abright |
|---|---|---|---|
| ABC | 5.27E−12 | 4E−10 | 4E−10 |
| ABC | 5.27E−12 | 4E−10 | 4E−10 |
| ABC | 5.27E−12 | 4E−10 | 4E−10 |
| ABX#1 | 9.65E−12 | 5E−09 | 5E−09 |
| ABX#1 | 9.65E−12 | 5E−09 | 5E−09 |
| ABX#1 | 9.65E−12 | 5E−09 | 5E−09 |
| ABX#1 | 9.65E−12 | 5E−09 | 5E−09 |
| ABX#2 | 8.42E−12 | 1.1E−09 | 1.1E−09 |
| ABX#2 | 8.42E−12 | 1.1E−09 | 1.1E−09 |
| ABX#2 | 8.42E−12 | 1.1E−09 | 1.1E−09 |
| ABX#2 | 8.42E−12 | 1.1E−09 | 1.1E−09 |
| ABX#3 | 6.38E−12 | 4E−10 | 4E−10 |
| ABX#3 | 6.38E−12 | 4E−10 | 4E−10 |
| ABX#3 | 6.38E−12 | 4E−10 | 4E−10 |
| ABX#3 | 6.38E−12 | 4E−10 | 4E−10 |
| ABX#4 | 2.38E−12 | 1E−10 | 1E−10 |
| ABX#4 | 2.38E−12 | 1E−10 | 1E−10 |
| ABX#4 | 2.38E−12 | 1E−10 | 1E−10 |
| ABX#4 | 2.38E−12 | 1E−10 | 1E−10 |
| ABX#5 | 7.4E−13 | 4E−11 | 4E−11 |
| ABX#5 | 7.4E−13 | 4E−11 | 4E−11 |
| ABX#5 | 7.4E−13 | 4E−11 | 4E−11 |
| ABX#5 | 7.4E−13 | 4E−11 | 4E−11 |
| NTC | 0 | 0 | 0 |

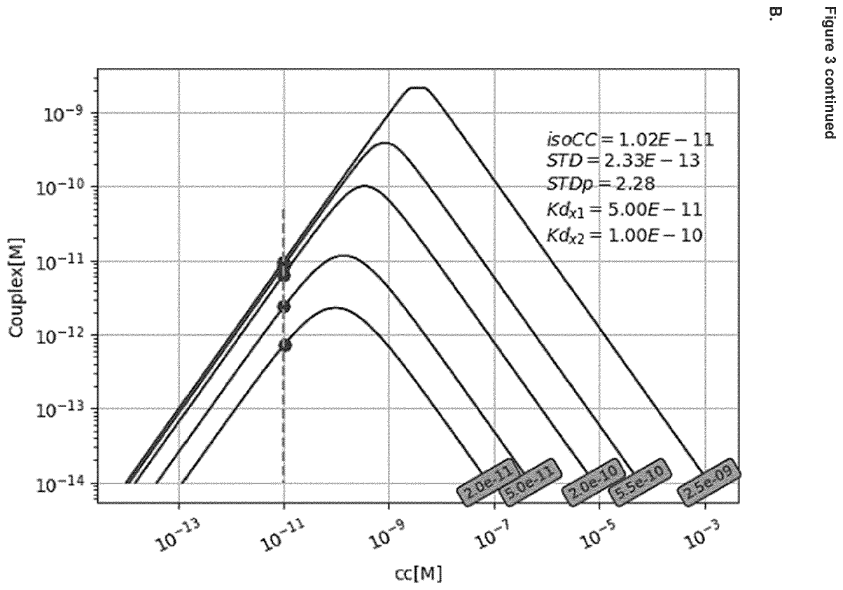

FIG. 3 depicts a graphical depiction of simulated isomolar titration data (see table 2 and 3) at 0.5 of active fraction. The preset concentration of target was 1.0E−11 M (isoCC). A. Data from Table 3. apparent Kds 1.0E−10 (Kdx1) and 1.0E−10 (Kdx2) are, respectively. The recursively minimized standard deviation of antigen concentration was 0.12% (STDp) or 1.16E−14 (STD). B. Data from Table 2., the recursively minimized standard deviation of antigen concentration was 2.28% (STDp) or 2.33E−13 (STD), where the (apparent) Kds are 5.0E−11 (Kdx1) and 1.0E−10 (Kdx2) respectively. These values are identical to the values of FIG. 2, reproducing the preset values. (A) describes the case when the active fraction is unknown, and the active concentration of antibody is falsely determined on the basis of labeled antibodies (the real concentration is reduced as the inactive antibodies are non-reactive). It is shown that after recursive minimization of the standard deviation of antigen concentration the isoCC is identical to the original isoCC, but the apparent Kd are not. (B) the control simulation. The antibody concentration is adjusted according to the active fraction, the original Kds are reproduced along with the original isoCC.

TABLE 4
Simulated isomolar titration data using corrected antibody x1 and
couplex concentrations at value of 0.5 of labeled fraction for determining
the apparent Kds of antibodies. Column headings are: ‘name’—sample
name, ‘couplex’—couplexes, ‘ableft’—concentration of
antibody x2, ‘right’—concentration of antibody x1, all concentrations
are in M. ABX means ‘antibody mix’ and they contain the concentration
of the two antibodies indicated. ‘ableft’ and ‘couplex’
values are adjusted as the labeled fraction is 0.5 compared to Table 1.

| name | couplex | ableft | abright |
|---|---|---|---|
| ABC | 3.53E−12 | 2E−10 | 4E−10 |
| ABC | 3.53E−12 | 2E−10 | 4E−10 |
| ABC | 3.53E−12 | 2E−10 | 4E−10 |
| ABX#1 | 4.855E−12 | 3E−09 | 5E−09 |
| ABX#1 | 4.855E−12 | 3E−09 | 5E−09 |
| ABX#1 | 4.855E−12 | 3E−09 | 5E−09 |
| ABX#1 | 4.855E−12 | 3E−09 | 5E−09 |
| ABX#2 | 4.38E−12 | 6E−10 | 1.1E−09 |
| ABX#2 | 4.38E−12 | 6E−10 | 1.1E−09 |
| ABX#2 | 4.38E−12 | 6E−10 | 1.1E−09 |
| ABX#2 | 4.38E−12 | 6E−10 | 1.1E−09 |
| ABX#3 | 3.53E−12 | 2E−10 | 4E−10 |
| ABX#3 | 3.53E−12 | 2E−10 | 4E−10 |
| ABX#3 | 3.53E−12 | 2E−10 | 4E−10 |
| ABX#3 | 3.53E−12 | 2E−10 | 4E−10 |
| ABX#4 | 1.59E−12 | 5E−11 | 1E−10 |
| ABX#4 | 1.59E−12 | 5E−11 | 1E−10 |
| ABX#4 | 1.59E−12 | 5E−11 | 1E−10 |
| ABX#4 | 1.59E−12 | 5E−11 | 1E−10 |
| ABX#5 | 5.65E−13 | 2E−11 | 4E−11 |
| ABX#5 | 5.65E−13 | 2E−11 | 4E−11 |
| ABX#5 | 5.65E−13 | 2E−11 | 4E−11 |
| ABX#5 | 5.65E−13 | 2E−11 | 4E−11 |
| NTC | 0 | 0 | 0 |

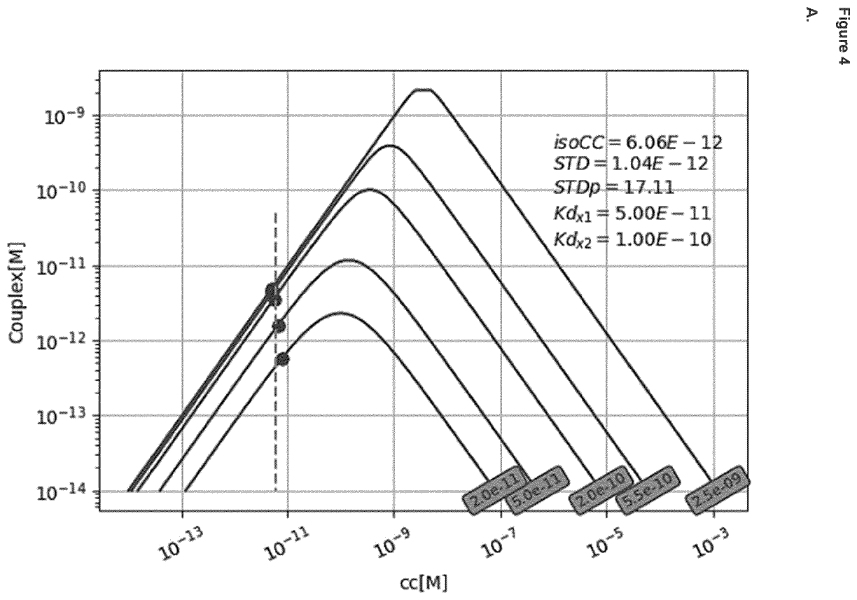

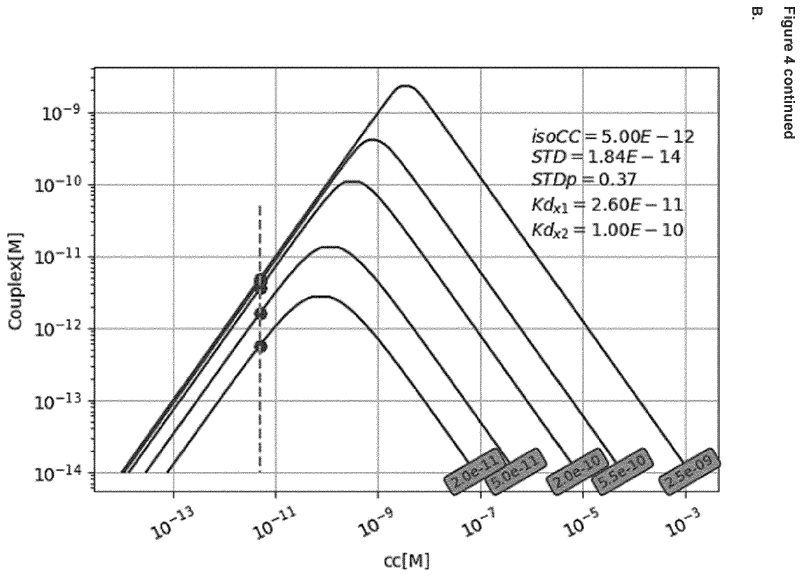

FIG. 4 depicts a graphical representation of the simulated isomolar titration data (see table 4) at 0.5 of labeled fraction. The preset concentration of the target was 1.0E−11 M (isoCC). FIG. 4A shoes that the apparent antibody and couplex concentrations were adjusted, based on simulation, and indicated antibody concentration were 2.0E−11, 5.0E−11, 2.0E−10, 5.5E−10 and 2.50E−9 (half amount as only the labeled fraction is detected). Sub-figure A. shows no recursive minimization. Kds 5.0E−11 (Kdx1) and 1.0E−10 (Kdx2) are, respectively. The standard deviation of antigen concentration was 17.11% (STDp) or 1.04E−12 (STD). FIG. 4B shows that after recursive minimization, the standard deviation of antigen concentration was 0.37% (STDp) or 1.84E−14 (STD) the apparent Kds 5.0E−11 (Kdx1) and 1.0E−10 (Kdx2) are, respectively, and the concentration of antigen (isoCC) was 5.0E−12. As the 0.5 of labeled fraction, the antigen concentration needs to be adjusted resulting in 1.0E−11 M, which identical with the original antigen concentration.

The simulations show that labeled ratio of antibodies, the active fraction of the antibody effect the absolute quantitative determination of the concentration of analyte, and they affect it differently. The active fraction has a self-compensating effect, apparent Kds will confound the effect of the active fraction, while the labeled ratio needs to be determined separately, and it needs to be numerically compensated in the absolute quantitative results. Numerical adjustment based on the labeled ratio of antibodies and the active fraction improves the accuracy of absolute quantitative determination of the concentration of analyte using the method.

### Example 4

The present example provides an example that determining the dissociation constant of binding agent(s), is affected by additional factors including the labeled ratio of binding agent(s), the label synthesis error, the active fraction of the binding agent(s) and the contamination with free labels. As shown in the present example the active fraction of the binding agent(s) and the contamination with free labels will be advantageously confounded in the determination of dissociation constant of antibody. As shown herein, preferably only the labeling ratio, label error of binding agents need to be independently determined to control and compensate for the precision of the method for determining the dissociation constant of antibody (which is, e.g., a compartmentalized bi-component method). This method is advantageous in the determination of the binding characteristics of the binding agent for the method of invention.

### Labeling Efficiency and Label Error

The ‘labeled fraction’ or ‘labeling efficiency’ or ‘labeled fraction’ of binding agents, such as antibodies, is an independent parameter for the determination of the absolute quantitative concentration of target analytes using the method. To mathematically prove b10 (absolute concentration of antibody 1), b20 (absolute concentration of antibody 2) and c12 (absolute concentration of the formed couplexes in the reaction), the following formula can be deployed:

$${b}_{10} ← {lf}_{1} {b}_{10}$$

$${b}_{20} ← {lf}_{2} {b}_{20}$$

$${e}_{11} ← {lf}_{1} {lf}_{2} {c}_{12} ,$$

where If1 and If2 are the labeled fraction for the two antibodies (first and second type of binding agents), respectively. If1 and If2 are independent variables and related to other parameters. Note that if the exact values of If1 and If2 are determined, it is easy to reverse this transformation, and reconstruct the absolute concentration of antibodies.

The label error, which can be considered as binding agents, e.g., antibodies, that are either unlabeled or comprise a faulty label, has identical analytical effects on the absolute quantification of the analyte and it can be derived in the same mathematical form.

### Active Fraction and Free Labels

The active fraction of the binding agents, e.g., antibodies, is a dependent parameter of the absolute quantitative determination of the concentration of a target analyte. To mathematically prove that b1 (free absolute concentration of antibody 1), b2 (free absolute concentration of antibody 2) and c12 (absolute concentration of the formed couplexes in the reaction), a (free absolute concentration of analyte), c1 and c2 are the absolute concentration of the bi-molecular complexes (couplexes) of the analyte and the respective antibodies b1 and b2, the following formula can be deployed:

$$k {d}_{1} = \frac{a ( {b}_{1} a {f}_{1} )}{{c}_{1}}$$

$$k {d}_{2} = \frac{a ( {b}_{2} a {f}_{2} )}{c 2}$$

$$k {d}_{1} = \frac{( {af}_{1} {b}_{1} ) {c}_{2}}{{c}_{12}}$$

$${kd}_{2} = \frac{( a {f}_{2} {b}_{2} ) {c}_{1}}{{c}_{12}} ,$$

where af1 and af2 are the active fractions for the two antibodies. After some rearrangement, equations can be transformed into equations that have the original chemical equilibrium terms on the right side and a constant modified term on the left side, proving that changing is the active fraction is self-compensating with respect to altering the measured apparent dissociation constant, and hence can be eliminated:

$$\frac{k {d}_{1}}{a {f}_{1}} \text{=:} {\text{?}}_{1} = \frac{a {b}_{1}}{{c}_{1}}$$

$$\frac{k {d}_{2}}{a {f}_{2}} \text{=:} {\text{?}}_{2} = \frac{a {b}_{2}}{c 2}$$

$$\frac{k {d}_{1}}{a {f}_{1}} \text{=:} {\text{?}}_{1} = \frac{{b}_{1} {c}_{2}}{{c}_{12}}$$

$$\frac{k {d}_{2}}{a {f}_{2}} \text{=:} {\text{?}}_{2} = \frac{{b}_{2} {c}_{1}}{{c}_{12}} .$$

It should be noted that these derived dissociation constants no longer conform to the original definition of the dissociation constant, however, represent the apparent effects of antibodies in the reactions. It is not possible to use the original chemical constants describing the chemical reaction, hence obtaining correct numerical values.

The presence of free labels, which can be considered as inactive antibodies, has identical analytical effects on the absolute quantification of the analyte as it can be derived in the same mathematical form.

### Example 5

In preferred embodiments the labeled ratio of binding agent(s) and/or the label synthesis error need to be independently determined to control and compensate for the precision of a compartmentalized bi-component method for determining the dissociation constant of binding agent(s). The present example provides an example of methods to determine the labeling ratio of the binding agent(s), the label synthesis error of binding agent(s). This method is advantageous in the determination of the binding characteristics of the binding agent for the method of invention determining the labeled fraction of the binding agent.

The labeling efficiency of the antibody is analyzed using the Agilent Protein 230 Chip on the 2100 Bioanalyzer from Agilent. For this, 4 μl of the labeled and unlabeled (as control) antibody solution were loaded according to the manufacturer's instructions. To determine the labeling efficiency, the concentration [ng/μl] of the light chain (lc; 26±3 kDa), of the heavy chain (hc; 54±4 kDa) and the labeled heavy chain (L; 75±5 kDa) is recorded. The labeled fraction (LE) is calculated using the concentration values as follows:

$$( 1 - {( \frac{h c}{h c + L} )}^{2} ) * 100$$

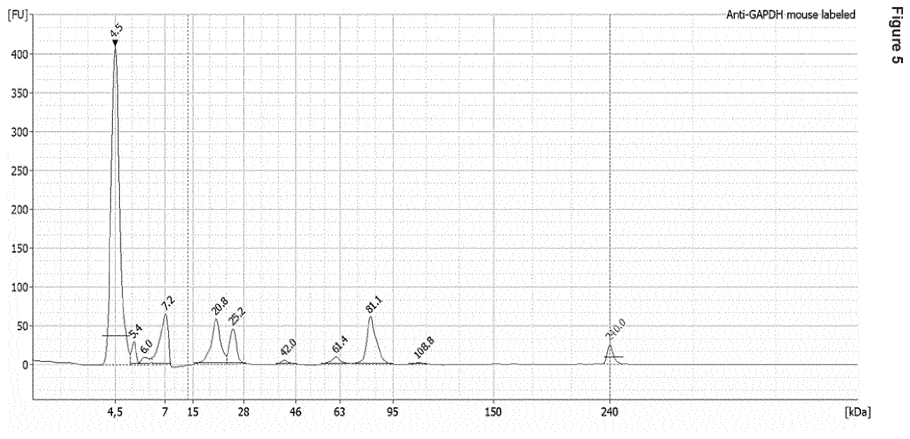

The FIG. 5 shows the Agilent Protein 230 Chip on the 2100 Bioanalyzer electrophoretogram of labeled antibody of anti-GAPDH. The labeled fraction was determined according to the text and was 97.6%.

The labeled antibody was purified using agarose A/G affinity matrix using several washing steps. The concentration of the labeled antibody is determined by digital PCR (dPCR) using the QIAcuity dPCR device. The method described elsewhere (see Example 1). Both the properly diluted last wash sample and the antibodies were measured. The free fraction of label was calculated by dividing concentration of the free label with the concentration of the labeled antibody. dPCR determined concentration of the free label (3.42E9 cp/μl) and the concentration of the labeled antibody (4.94E10 cp/μl), the fraction of free label was calculated according to the present Example and was 14.4%.

### Label Error

Labels were synthesized according to proprietary DNA sequences of Actome GmbH for both BL and P8 labels (described in Example 1), these labels are amplified using a dPCR protocol (described in Example 1). dPCR enables to determine the exact amplifiable/detectable amount of the labels. For all reactions, 100 μL aliquots of label samples and standards were pipetted into microplate wells and 100 μL of OliGreen working solution was added to each well. OliGreen working solution was prepared fresh by diluting OliGreen concentrated reagent 1:200 with TE as described by the manufacturer. Samples were incubated for approximately 5 minutes at room temperature and their fluorescence determined using a Bio-Tek Instruments FL600 fluorescent plate reader with a 485 nm, 20 nm bandwidth, excitation filter and 530 nm, 25 nm bandwidth emission filter. The sensitivity setting was varied as needed and the data collected from the top with a 3 mm probe using static sampling with a 0.35 second delay, 50 reads per well.

The difference of the two method measurements defines the label synthesis error.

### Example 6

The present example shows the determination of dissociation constants (Kd) of antibodies considering the labeled ratio, label synthesis error and the active fraction of binding agent(s), see Example 3. Preferably, a compartmentalized bi-component method using the labeled ratio and the label synthesis error of binding agent(s) is used to determine the apparent dissociation constant of the binding agent(s), thereby providing an advantageous, inherent, self-compensation for the active fraction of the binding agent(s) and the contamination with free labels to provide the dissociation constant of antibodies for the unbiased determination of absolute quantities in single-cells. This method is advantageous in the determination of the binding characteristics of the binding agent for the method of invention determining the labeled fraction of the binding agent.

The antibodies conjugated with DNA amplifiable labels using a proprietary procedure of Actome GmbH, Germany. The achieved labeling efficiencies were between 80-100% and were confirmed by SDS-PAGE electrophoresis (Example 5). The antibody preparations have 3% free labels, 25% labeling errors (see Example 5). Trastuzumab (TTZ) and pertuzumab (PTZ) are recombinant humanized monoclonal antibodies, both targeting extracellular regions of the HER2 tyrosine kinase receptor and having no overlapping epitopes. The conjugated antibodies have two different labels-trastuzumab BL, and pertuzumab P8 labels.

The bi-component assay used is similar to that described in WO 2016/083793 A1, including herein as a reference. Briefly, BT-474 cells optional cross-linked, in 5 mM BS3 in PBS for 1 hour at 4 C. BT-474 cell concentrations were adjusted to 1×10e7 cells/ml and was lysed in 100 μL of proprietary lysis buffer supplied by Actome GmbH, Germany and the lysate was incubated with the antibodies overnight at different concentration of both antibodies targeting the concentration of 1.00E−09, 2.00E−10, 4.00E−11, 8.00E−12 and 1.60E−12 M, respectively.

The incubated lysate was diluted in PBS to achieve a 0.15 lambda concentration in the final dPCR for the higher concentration of antibody used. Usually, antibody concentrations are equal in the ABX. A negative control containing only labeled trastuzumab and pertuzumab antibodies was also set up using a concentration of 4.00E−11 M of antibodies (antibody binding control).

QIAGEN's QIAcuity Probe Mastermix (41 μL) was added and the samples (0.5 μL lysed bulk) were loaded onto a QIAcuity Nanoplate 26 k 24-well plate. Digital PCR was performed using a QIAGEN QIAcuity Digital PCR System with cycling parameters according to the PCR conditions of hot-start 95° C. for 2 min, cycling 40 times, denaturing 95° C. for 15 sec, annealing 58° C. for 30 sec. Imaging conditions P8 label-FAM green channel, 500 ms integration time, gain 6 and BL label-HEX yellow channel, 400 ms integration time, gain 6.

From the dPCR the measured raw dPCR parameters (different positivity ratios) are obtained, as the concentration of antibodies (single color positivity ratio) and the double color positivity ratio (confounded statistical overlaps and bi-component complexes, called couplexes). From these three parameters of each individual measurement and using the bi-component assay theory (see WO 2020/260277 A1) the concentration of couplexes is obtained. Briefly, the resulting fluorescence readouts of compartments are thresholded and clustered into empty, single positive and double positives groups. dPCR evaluation is based on general dPCR theory (Basu, A. S. (2017) Digital Assays Part 15 I: Partitioning Statistics and Digital PCR. doi: 10.1177/2472630317705680) providing the single color-based determination of the number of positive compartments (antibody A, B), the number of double-colored compartments and the number of empty compartments. The evaluation of the reaction is based on the partitioning of the labels in the dPCR reactions. The ordinary dPCR evaluation determines the absolute number of labels in each reaction, which—in turn—can be used to calculate the number of double-positive droplets (the spontaneous overlap of labels) based on the statistical probability of co-compartmentalization (Poisson background). The Poisson background was compared to the number of detected double-positive droplets (including couplexes) and a statistical model was developed to calculate the number of molecular complexes explaining the number of double-positive droplets over the Poisson background. The concentration of couplexes was determined, multiplied by the dilution factor previously obtained to get the molar concentration of couplexes in the binding reaction. The molar concentration of antibodies was also similarly determined, the concentration were obtained from the direct reading of the dPCR reaction and similarly applying the dilution factor of the binding reaction. For a given antibody concentration four parallel experiments were carried out. ABC control has an expected zero couplex value hence its deviation from zero shows evaluation (clustering) and other biases and the non-zero couplexes of ABC, if measured, were used to normalize all measurements.

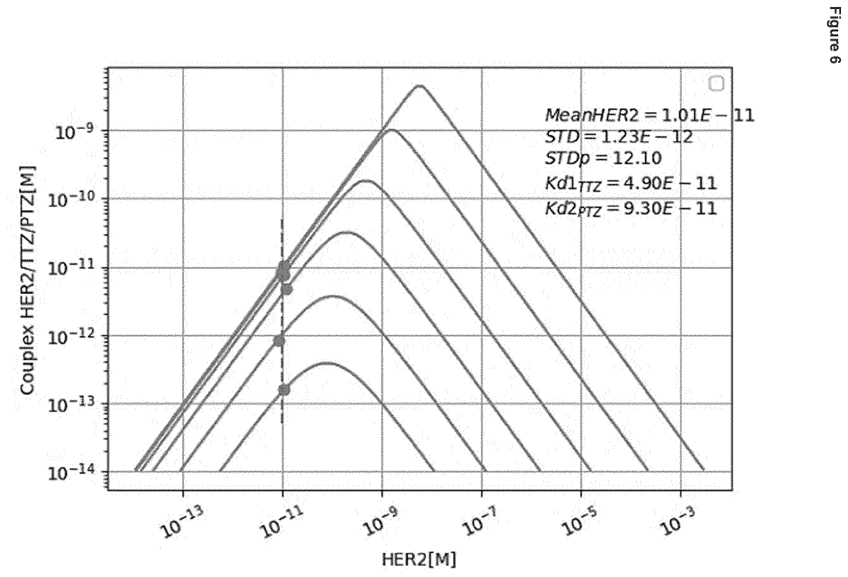

The FIG. 6 shows the calculated calibration curves for each sample. The calibration curves are derived using a method similar to the method described in WO 2020/260277 A1, however, with the adjusted binding characteristics. Using the separately determined labeling ratio and label error of the antibodies a corrected calibration curve was with the corrected values of single and double positive values of dPCR resulting in the corrected concentration of antibodies and couplexes.

The apparent dissociation constants of antibodies were calculated recursively minimizing the standard deviation of the recalculated HER2 concentrations for all measurement under the assumption of that all samples contains the same concentration of HER2.

FIG. 6 is a graphical depiction of the determination of the apparent dissociation constants of trastuzumab and pertuzumab. The samples had isomolar concentrations, however the exact concentration of HER2 was unknown (the sample was obtained from lysed HER2 cells), and the concentration of the antibodies varied (5.5E−9, 1.50E−9, 4.0E−10, 1.2E−10, 3.5E−11 and 1.0E−11 for antibodies). The lines represent calibration curves of HER2 specific bi-component assay similar to the method described in WO 2020/260277 A1 assay at these antibody concentrations and apparent Kds. The latter was determined minimizing the SD of the recalculated HER2 concentrations for all measurement under the assumption of that all samples contains the same concentration of HER2. The molar HER2 concentration was designated as MeanHER2, STD and pSTD were the standard deviation of measurement, and its percentage of mean, respectively. The dotted lines depict the means of HER2 measurements at different antibody concentrations. Finally, the dissociation of antibodies (Kd1TTZ and Kd2PTZ) are recursively determined to be to be 2.9E−11 and 9.3E−11, respectively.

### Example 7

This method is advantageous in the determination of the RNA content of the sample concurrently with the determination of absolute amount of target analytes using the method of invention.

### RNA Detection Concurrently

To carry out RNA detection concurrently, the DNA-dependent DNA polymerase activity of MMLV reverse transcriptases is exploited, described as SMART library prep protocols (Clontech—switching mechanism at 5′ end of RNA template). Briefly, the MMLV reverse transcriptase enzyme switches at the end of the RNA based DNA synthesis initiated from the 3′ end of the mRNA, to a TSO (template switching DNA oligonucleotide) (which in turn can introduce a 5′ PCR primer site—commonly referred as PCR handle) and together with the introduction of a similar PCR handle at 3′ end at the initiation of the first strand synthesis, the handles form a universally amplifiable amplicon. Briefly, MMLV reverse transcriptase terminal transferase activity adds a few additional nucleotides (mostly deoxycytidine) to the 3′ end of the newly synthesized cDNA strand. These deoxycytidine bases form the annealing site for the template switching (TS) oligo (TSO). The reverse transcriptase recognizes this new template strand, formed by cellular RNA to the TSO, and continues synthesis of the second strand incorporating a universal sequence, namely a PCR handle. Single-cell RNA-seq protocols applied this concept [Ramsköld, D., Luo, S., Wang, Y. C., Li, R., Deng, Q., Faridani, O. R., Daniels, G. A., Khrebtukova, I., Loring, J. F., Laurent, L. C., et al. (2012). Full-length mRNA-seq from single-cell levels of RNA and individual circulating tumor cells. Nat. Biotechnol. 30, 777-782].

Template Switching Oligo (TS) Structure. The simplest version of TSO is a DNA oligo sequence that carries 3 riboguanosines (rGrGrG) at its 3′ end [Zhu Y Y, Machleder E M, et al. (2001) Reverse transcriptase template switching: a SMART approach for full-length cDNA library construction Biotechniques, 30(4):892-897.]. The complementarity between these consecutive rG bases and the 3′ dC extension of the cDNA molecule enables template switching [Turchinovich A, Surowy H, et al. (2014) Capture and Amplification by Tailing and Switching (CATS). An ultrasensitive ligation-independent method for generation of DNA libraries for deep sequencing from picogram amounts of DNA and RNA. RNA Biol, 11(7):817-828.]. An approach was reported reducing library background and thereby improve cDNA yield by incorporating isomeric nucleotides into the TS oligo [Kapteyn J, He R, et al. (2010) Incorporation of non-natural nucleotides into template-switching oligonucleotides reduces background and improves cDNA synthesis from very small RNA samples. BMC Genomics, 11:413.]. In this study, 2 modified bases, iso-dC and iso-dG, were appended to the 5′ end of the TS oligo forming hydrogen bonds with each other but not with naturally occurring C and G nucleotides effectively locking the 5′ end of the TSO demonstrating efficacy in minimizing the concatenation of TS oligos, which typically results from cycles of reverse transcriptase activity [Saliba A E, Li L, et al. (2016) Single-cell RNA-seq ties macrophage polarization to growth rate of intracellular Salmonella. Nat Microbiol, 2:16206.].

MCF7 (ATCC® HTB-22) cells were cultured in DMEM, GlutaMAX Supplement (31966021, Gibco) supplemented with 10% FBS (10270106, Gibco) and 1% Pen/Strep (15140122, Gibco). Cells were cultured until ~90% confluency in a cell culture incubator (Heracell™ 150i CO2 Incubator, 50116048, Thermo Scientific™) under a 5% CO₂ atmosphere at 37° C. Cells were harvested scraping. The cells were washed twice with DPBS (14040133, Gibco) and counted (Countess® ∥ Automated Cell Counter, Invitrogen™) including live/dead staining with trypan blue (T10282, Invitrogen™).

The antibodies conjugated with DNA amplifiable labels using a proprietary procedure of Actome GmbH, Germany. The achieved labeling efficiencies were between 80-100% and were confirmed by SDS-PAGE electrophoresis. The antibody preparations have 3% free labels, 25% labeling errors (see Example 5). Trastuzumab (TTZ) and pertuzumab (PTZ) are recombinant humanized monoclonal antibodies, both targeting extracellular regions of the HER2 tyrosine kinase receptor and having no overlapping epitopes. The conjugated antibodies have two different labels-trastuzumab BL, and pertuzumab P8 labels. Dissociation constants of antibodies were determined according to Example 6 and were TTZ=0.49E−10 M and PTZ=0.93E−10, respectively.

The Immediate Drop-on-demand Technology (I.DOT One; Dispendix, Stuttgart, Germany) with I.DOT PURE plates 90 μm orifice (Dispendix, Stuttgart, Germany) was used to dispense 0.5 μl LBTW into a 384-well V-bottom plate (0030623304, Eppendorf). Prior to dispensation, the I.DOT was calibrated for the applied liquids to ensure reliable dispensation. The liquid class ‘H2O’ was used to dispense the diluted crude cell lysate (‘cl’) or DPBS.

In case of non-lysed cells, the cell dispensing procedure was performed as described earlier (T. Groß, C. Jeney, D. Halm, G. Finkenzeller, G. B. Stark, R. Zengerle, P. Koltay, S. Zimmermann, Characterization of CRISPR/Cas9 RANKL knockout mesenchymal stem cell clones based on single-cell printing technology and emulsion coupling assay as a low-cellularity workflow for single-cell cloning, BioRxiv. (2020) 1-19. https://doi.org/10.1101/2020.08.17.253559.). Briefly, using the F.SIGHT single-cell dispenser (CYTENA GmbH, Freiburg, Germany), an improved version of the single-cell printer (SCP) (A. Gross, J. Schöndube, S. Niekrawitz, W. Streule, L. Riegger, R. Zengerle, P. Koltay, Single-Cell Printer: Automated, On Demand, and Label Free, J. Lab. Autom. 18 (2013) 504-518. https://doi.org/10.1177/2211068213497204), both MCF7 and BT-474 cell concentrations were adjusted to 1×106 cells/ml and loaded into a Dispensing Cartridge (CYTENA GmbH, Freiburg, Germany). The settings for MCF7 cells were 10 to 25 μm of cell size (BT-474:10 to 30 μm) and 0.5 to 1 of roundness (same for BT-474) (FIG. 1a, S3a and S3b), respectively. The F.SIGHT can reliably dispense single cells in minimal liquid volumes. The single-cell dispensation efficiency (successful single-cell isolation events divided by targeted events) is usually around 90%, and is additionally controlled by cell images unambiguously assigned to each dispensation event. Thus, other than single-cell dispensation events like doublets or empty droplets can be excluded.

The samples (0.5 μL of single-cell samples) were added mastermix was prepared as follows: 1× qScript XLT 1-Step RT-qPCR ToughMix (95132, Quantabio) containing (1.3 mM GTP, 1.3 U/μL RNAse inhibitor, 12.5 μM TSO, PCR handle-tagged-random hexamer) and loaded onto four Naica® Crystal Digital PCR System Sapphire Chips reactions and read with the Prism3 reader (Stilla Technologies, Villejuif, France). Digital PCR was performed with cycling parameters of (cycling 5 times, incubating 42° C. for 10 mins and 25° C. for 15 sec, followed by PCR conditions of hot-start 95° C. for 2 min, cycling 40 times, denaturing 95° C. for 15 sec, annealing 58° C. for 30 sec). Imaging conditions P8 label—FAM green channel, 500 ms integration time, gain 6 and BL label-HEX yellow channel, 400 ms integration time, gain 6.).

Evaluate the bi-component assay signal as described (Example 6).

After cycling remove the Sapphire chip from the cycler and remove the blue cap to recover the emulsion, using chloroform or 20% perfluorooctanol (PFO) in hydrofluoroether (HFE), break the emulsion and purify the cDNA library. Briefly, 125 μL of recovery agent (20% PFO in HFE), 55 μL of GITC Buffer (5 M GITC, 25 mM EDTA, 50 mM Tris-HCl pH 7.4) and 5 μL of 1 M DTT was added to each separate aliquot of 50 μL emulsion and incubated on ice for 5 min. 99 μL of Ampure XP beads was added to the aqueous phase and incubated for 10 min. Ampure beads were pelleted on a Nd magnet and washed twice with 80% EtOH. Elution was performed in 30 μL of T8.5-DTT-Tween (10 mM DTT, 0.1% Tween-20 in T8.5 (10 mM Tris-HCl, pH 8.5)).

Linear PCR amplification was performed (40 μL library, 50 μL 2×KAPA HiFi (Roche), 10 μL 10 μM TSO-PCR handle primer). PCR cycling was performed according to (95° C. for 3 min, 13 cycles of [98° C. for 20 s, 63° C. for 20 s, 72° C. for 3 min.], 72° C. for 5 min) followed by a final hold at 4° C. 2 μL of 1 M DTT was added and a 0.6× Ampure XP purification was performed according to manufacturer's recommendations.

Final sequencing library was prepared according to the following customized NEB Ultra II FS protocol (NEB E7805S). 80 ng of amplified cDNA was fragmented in Ultra II fragmentation mix (26 μL of amplified cDNA, 7 μL of NEBNext Ultra II FS Reaction Buffer, 2 μL of NEBNext Ultra II FS Enzyme Mix) using thermocycling protocol: 37° C. for 10 min, 65° C. for 30 min, and a final hold at 4° C. 15 μL of T8.5 was added and a 0.8× Ampure purification was performed according to manufacturer's recommendation and eluted in 50 μL. Fragmented library was adapter ligated using NEBNext Ultra II adapter ligation mix (35 μL of fragmented library, 30 μL of NEBNext Ultra II Ligation Master Mix, 1 μL of NEBNext Ligation Enhancer, 2.5 μL of NEBNext Adapter for Illumina) at 20° C. for 15 min, with 4° C. hold. 28.5 μL of T8.5 was added and a 0.8× Ampure purification was performed according to manufacturer's recommendation and eluted in 30 μL. Eluted library was amplified in PCR master mix (50 μL 2×KAPA HiFi, 10 μL 10 μM Hy-i7 primer, 10 μL 10 μM Hy-i5 primer, 30 μL eluted library) in the following thermocycling program: 95° C. for 3 min, 13 cycles of (98° C. for 20 s, 64° C. for 30 s, 72° C. for 30 s), 72° C. for 5 min, and a final hold at 4° C. Sequencing-ready library was purified using a 0.8× Ampure purification and eluted in 30 μL of T8.5.

Libraries were sequenced on Illumina NextSeq2000 systems using 50 cycles for read 1 (3′ end of cDNA), 10 cycles for index 1 (sample index, custom i7 read primer), 10 cycles for index 2 (sample index), and 58 cycles for read 2 (5′ end of cDNA).

Libraries were data processed using a custom pipeline, but according to the main guideline of scRANseq. Briefly, reads were mapped and cell-demultiplexed using STARsolo (Kaminow B, Yunusov D, Dobin A. 2021. GenomicSuperSignature: interpretation of RNA-seq experiments through robust, efficient comparison to public databases. Bioinformatics (Oxford, England) 1: e55. DOI: https://doi.org/10.1101/2021.05.05.442755), cells were filtered on expression of a maximum of 4000 genes. The filtered expression matrix was scaled to total counts and log-normalized.

### Example 8

This method is advantageous in the determination of absolute amount of target analytes using the method of invention forming physically separated first compartments.

Determining the absolute amount of the target analyte, the first compartmentalization is carried out into first partitions, wherein the compartmentalization is physical, the binding reaction between the cells and the binding agents is preferably carried out at high target analyte-concentration exerted by a low volume encompassing each single-cell. Preferably, the cells are lysed during the first compartmentalization. Each first compartment is second-compartmentalized, and a suitable sensitive method is used to count the single target analyte molecules. Preferably, the concentration of the target in the first compartment is determined using the binding characteristics, and finally results in the determination of the absolute amount/number of the target analyte per single-cell, as described in the present Example.

Providing a single-cell sample comprising, incubation the sample with two target specific antibodies, wherein the target and the antibodies form couplexes, during compartmentalization said of single-cell sample into a plurality of first partitions and complementing each first partition comprising a single cell with a lysis buffer. During the compartmentalized bi-component method (second partitioning) the conditions of the couplexes in the first partitions are determined.

MCF7 (ATCC® HTB-22) cells were cultured in DMEM, GlutaMAX Supplement (31966021, Gibco) supplemented with 10% FBS (10270106, Gibco) and 1% Pen/Strep (15140122, Gibco). Cells were cultured until ~90% confluency in a cell culture incubator (Heracell™ 150i CO2 Incubator, 50116048, Thermo Scientific™) under a 5% CO₂ atmosphere at 37° C. Cells were harvested scraping. The cells were washed twice with DPBS (14040133, Gibco) and counted (Countess® ∥ Automated Cell Counter, Invitrogen™) including live/dead staining with trypan blue (T10282, Invitrogen™).

The antibodies conjugated with DNA amplifiable labels using a proprietary procedure of Actome GmbH, Germany. The achieved labeling efficiencies were between 80-100% and were confirmed by SDS-PAGE electrophoresis. The antibody preparations have 3% free labels, 25% labeling errors (see Example 5). Trastuzumab (TTZ) and pertuzumab (PTZ) are recombinant humanized monoclonal antibodies, both targeting extracellular regions of the HER2 tyrosine kinase receptor and having no overlapping epitopes. The conjugated antibodies have two different labels-trastuzumab BL, and pertuzumab P8 labels. Dissociation constants of antibodies were determined according to Example 6 and were TTZ=0.49E−10 M and PTZ=0.93E−10, respectively.

Cells were encapsulated in HFE-7500 Novac oil with EA-008 surfactant (RAN Biotech) using droplet generation chip (LabSmith) on the Onyx microfluidics platform (Droplet Genomics) in (PBS, 25 mM DTT, 15% Optiprep, 1.3 U/μL RNAse inhibitor, complete Protease Inhibitor Cocktail Tablets 1×cc, 4.4% PEG-8000) and picoinjectioned using the Onyx system achieving 1×LBTW with labeled Trastuzumab (TTZ) and pertuzumab (PTZ) (concentration of 1.e−11 M for both) in the first partitions. The resulting emulsion was collected in aliquots of 50 μL total volume and incubated 4 C overnight.

Droplet dispensing was performed at droplet concentrations were adjusted to 1×10⁶ droplet/ml delivering single first compartments into 96-well microplate wells. QIAGEN's QIAcuity Probe Mastermix (42 μL) was added to a 96-well microplate and a single droplet was dispensed into the wells and after mixing loaded onto a QIAcuity Nanoplate 26 k 24-well plate. A negative control containing only labeled trastuzumab and pertuzumab antibodies was also set up using a concentration of 4.00E−11 M of antibodies (antibody binding control). Digital PCR was performed using a QIAGEN QIAcuity Digital PCR System with cycling parameters according to the PCR conditions of hot-start 95° C. for 2 min, cycling 40 times, denaturing 95° C. for 15 sec, annealing 58° C. for 30 sec. Imaging conditions P8 label-FAM green channel, 500 ms integration time, gain 6 and BL label—HEX yellow channel, 400 ms integration time, gain 6.

The count of couplexes and the count of antibodies were obtained as described elsewhere (Example 6). The count of antibodies expected on average of 4320 (antibody label ratio and label error corrected) copies per QIAcuity Nanoplate 26 k 24-well plate reactions and the detected couplexes were on average 40 (antibody label ratio and label error corrected). If the antibody concentration was 4.e−11 M then the detected 4320 copies of antibodies corresponds to 0.18 nL sizes droplets, hence the concatenation of couplexes (on average) is 3.7e−13 M. Based on the concentration of antibodies, the predetermined binding characteristics including the Kd of antibodies (exemplary Kds are PTZ=7.4E−11 and TTZ=1.2E−10, apparent kd determined see Example 6) a calibration curve can be constructed. However, couplexes can be ranged from 10 to 150, correspondingly 9.2e−14 and 1.3e−12 M, repeatedly. The calculated concentration of HER2 protein target is 4.4e−12 M (on average), which is in 0.18 nL is 476 copies, which is 476 copies of HER2 per cell (on average), ranging from 114 to 1992 copies of HER2 per cell.

### Example 9

This method is advantageous in the determination of absolute amount of target analytes using the method of invention forming physically separated first compartments, but using protein labels instead of nucleic acid labels,

### Protein Labeling

One exemplary embodiment of carrying out the invention using protein labels, antibody-capture agents are attached to the surface of 2.7-μm-diameter paramagnetic beads, each containing 250,000 attachment sites. Typically, 500,000 beads will be added to a 100-μL sample. At low target concentrations when the number of beads is greater than the number of target molecules in a sample, each bead will capture 0 or 1 target molecule. The fraction of beads that capture a target molecule in a given sample follows the Poisson distribution. Single beads are trapped in femtoliter-sized wells on a Simoa Disc, which allows for a ‘digital’ readout of each individual bead to determine if it is bound to the target analyte or not.

The beads were washed to remove nonspecifically bound proteins and are incubated with two detector antibodies labeled with β-galactosidase and alkaline phosphatase, respectively. In this manner, each bead that has captured a single protein molecule is detected with two enzymes. Beads that do not capture a molecule remain label-free. The beads are loaded into arrays of 216,000 femtoliter-sized wells, each of which hold no more than one bead. Beads are added in the presence of substrate, and wells are subsequently sealed with oil and imaged. If a target analyte has been captured (that is, an immune complex has formed), then the substrate will be converted to a fluorescent product by the captured enzyme label. The reaction is evaluated according to the bi-component evaluation concept (Example 6) reading single and double positive compartments along with counting all compartments.

### Example 10

For larger numbers of target analytes, the readout/analysis of the bi-component method may be carried out by nucleic acid sequencing, such as NGS, in which case the labels should be nucleic acid sequences, that comprise a sequence that is unique for each type of binding agent. In embodiments molecule-specific barcodes (UMI) are added additionally to the label of each binding agent, either during the initial binding agent labelling reaction or at a later time point to each partition. The example describes the detection of targets using the method of invention using NGS as read out method. This method is advantageous in the determination of absolute amount of target analytes using a method of the invention determining the absolute condition of first compartments using next generation sequencing methods.

### NGS Readouts

MCF7 (ATCC® HTB-22T) cells were cultured in DMEM, GlutaMAX Supplement (31966021, Gibco) supplemented with 10% FBS (10270106, Gibco) and 1% Pen/Strep (15140122, Gibco). Cells were cultured until ~90% confluency in a cell culture incubator (Heracell™ 150i CO2 Incubator, 50116048, Thermo Scientific™) under a 5% CO2 atmosphere at 37° C. Cells were harvested scraping. The cells were washed twice with DPBS (14040133, Gibco) and counted (Countess® ∥ Automated Cell Counter, Invitrogen™) including live/dead staining with trypan blue (T10282, Invitrogen™).

The antibodies conjugated with DNA amplifiable labels using a proprietary procedure of Actome GmbH, Germany. The achieved labeling efficiencies were between 80-100% and were confirmed by SDS-PAGE electrophoresis. The antibody preparations have 3% free labels, 25% labeling errors (see Example 5). Trastuzumab (TTZ) and pertuzumab (PTZ) are recombinant humanized monoclonal antibodies, both targeting extracellular regions of the HER2 tyrosine kinase receptor and having no overlapping epitopes. The conjugated antibodies have two different labels-trastuzumab BL, and pertuzumab P8 labels with 10 bp UMIs incorporated. Dissociation constants of antibodies were determined according to Example 6 and were TTZ=0.49E−10 M and PTZ=0.93E−10, respectively.

The Immediate Drop-on-demand Technology (I.DOT One; Dispendix, Stuttgart, Germany) [50, 51] with I.DOT PURE plates 90 μm orifice (Dispendix, Stuttgart, Germany) was used to dispense 0.5 μl LBTW into a 384-well V-bottom plate (0030623304, Eppendorf). Prior to dispensation, the I.DOT was calibrated for the applied liquids to ensure reliable dispensation. The liquid class ‘H2O’ was used to dispense the diluted crude cell lysate (‘cl’) or DPBS.

In case of non-lysed cells, the cell dispensing procedure was performed as described earlier (T. Groß, C. Jeney, D. Halm, G. Finkenzeller, G. B. Stark, R. Zengerle, P. Koltay, S. Zimmermann, Characterization of CRISPR/Cas9 RANKL knockout mesenchymal stem cell clones based on single-cell printing technology and emulsion coupling assay as a low-cellularity workflow for single-cell cloning, BioRxiv. (2020) 1-19. https://doi.org/10.1101/2020.08.17.253559.). Briefly, using the F.SIGHT single-cell dispenser (CYTENA GmbH, Freiburg, Germany), an improved version of the single-cell printer (SCP) [43], both MCF7 and BT-474 cell concentrations were adjusted to 1×10e6 cells/ml and loaded into a Dispensing Cartridge (CYTENA GmbH, Freiburg, Germany). The settings for MCF7 cells were 10 to 25 μm of cell size (BT-474:10 to 30 μm) and 0.5 to 1 of roundness (same for BT-474) (FIG. 1a, S3a and S3b), respectively. The F.SIGHT can reliably dispense single cells in minimal liquid volumes. The single-cell dispensation efficiency (successful single-cell isolation events divided by targeted events) is usually around 90%, and is additionally controlled by cell images unambiguously assigned to each dispensation event. Thus, other than single-cell dispensation events like doublets or empty droplets can be excluded.

The master mix was prepared as follows: 1× PerfeCTa® qPCR ToughMix® (Quantabio) containing (an optimized setup of linkage PCR primers, obtained by state of art optimization methods) using primers with cell specific barcodes and loaded onto a Naica® Crystal Digital PCR System Sapphire Chip reaction and read with the Prism3 reader (Stilla Technologies, Villejuif, France). The linkage PCR was performed with cycling parameters of (PCR conditions of hot-start 95° C. for 2 min, cycling 240 times, denaturing 95° C. for 15 sec, annealing 58° C. for 30 sec). Imaging conditions P8 label—FAM green channel, 500 ms integration time, gain 6 and BL label-HEX yellow channel, 400 ms integration time, gain 6.).

Evaluate the bi-component signal as described (Example 6). The number of amplification compartments were recorded, or a quantified tracer linkage PCR amplicon was used to calculate it. After cycling remove the Sapphire chip from the cycler and remove the blue cap to recover the emulsion, using chloroform or 20% perfluorooctanol (PFO) in hydrofluoroether (HFE), break the emulsion and purify the cDNA library. Briefly, 125 μL of recovery agent (20% PFO in HFE), 55 μL of GITC Buffer (5 M GITC, 25 mM EDTA, 50 mM Tris-HCl pH 7.4) and 5 μL of 1 M DTT was added to each separate aliquot of 50 μL emulsion and incubated on ice for 5 min. 99 μL of Ampure XP beads was added to the aqueous phase and incubated for 10 min. Ampure beads were pelleted on a Nd magnet and washed twice with 80% EtOH. Elution was performed in 30 μL of T8.5-DTT-Tween (10 mM DTT, 0.1% Tween-20 in T8.5 (10 mM Tris-HCl, pH 8.5)).

Linear PCR amplification was performed (40 μL library, 50 μL 2×KAPA HiFi (Roche), 10 μL 10 μM TSO-PCR handle primer). PCR cycling was performed according to (95° C. for 3 min, 13 cycles of [98° C. for 20 s, 63° C. for 20 s, 72° C. for 3 min.], 72° C. for 5 min) followed by a final hold at 4° C. 2 μL of 1 M DTT was added and a 0.6× Ampure XP purification was performed according to manufacturer's recommendations.

The library was adapter ligated using NEBNext Ultra II adapter ligation mix (35 μL of fragmented library, 30 μL of NEBNext Ultra II Ligation Master Mix, 1 μL of NEBNext Ligation Enhancer, 2.5 μL of NEBNext Adapter for Illumina) at 20° C. for 15 min, with 4° C. hold. 28.5 μL of T8.5 was added and a 0.8× Ampure purification was performed according to manufacturer's recommendation and eluted in 30 μL. Eluted library was amplified in PCR master mix (50 μL 2×KAPA HiFi, 10 μL 10 μM Hy-i7 primer, 10 μL 10 μM Hy-i5 primer, 30 μL eluted library) in the following thermocycling program: 95° C. for 3 min, 13 cycles of (98° C. for 20 s, 64° C. for 30 s, 72° C. for 30 s), 72° C. for 5 min, and a final hold at 4° C. Sequencing-ready library was purified using a 0.8× Ampure purification and eluted in 30 μL of T8.5.

Libraries were sequenced on Illumina NextSeq2000 systems using 50 cycles for read 1, 10 cycles for index 1 (sample index, custom i7 read primer), 10 cycles for index 2 (sample index), and 58 cycles for read 2.

Libraries were data processed using a custom pipeline. Briefly, cells, UMIs were deconvoluted and following the description of such library deconvolutions the droplets were deconvoluted as described in WO 2020/260277 A1. Briefly, deconvolution of droplets is achieved on the basis of unique pairing between UMIs per droplet during the linkage PCR step, such pairings make droplet local pairs of UMIs on a random basis and such network of pairs are highly unique, defining unequivocally the droplet, enabling the reconstruction of the droplet content referring the type of the antibodies present. Based on the content of the droplet the distribution of the antibodies can be derived, so droplets with one type of antibody and droplets with both antibodies present can be counted. The overall number of droplets is derived by direct counting, or using tracer concepts. The droplet information gained can be evaluated using methods described for dPCR readouts (Example 6).

1. A method for determining the concentration of at least one target analyte in a sample comprising:
a. providing at least a binding agent of a first type and a binding agent of a second type, each comprising a label that is unique for the type of binding agent, wherein the binding agent of a first type and the binding agent of a second type specifically bind to a first target analyte,
b. determining the binding characteristics of the binding agent of a first type and the binding agent of a second type to the first target analyte,
c. providing a sample comprising cells at a known concentration,
d. bringing the binding agent of a first type and the binding agent of a second type into contact with the sample, wherein the binding agent of a first type and the binding agent of a second type bind to the first target analyte forming an analyte-binding agent complex (couplexes),
e. compartmentalizing single cells of the sample into a plurality of first partitions,
f. complementing each first partition comprising a single cell with a lysis buffer, thereby lysing the single cell in each of the first partitions,
g. compartmentalizing single analyte-binding agent complexes (couplexes) into a plurality of second partitions,
h. performing a bi-component detection method, thereby determining the number and/or absolute concentration of couplexes and binding agents in the second partition, and
i. determining the absolute concentration of the first target analyte per cell in the first partition by taking into account the initial concentration of the cells in the sample in step a., the binding characteristics determined in step b. and the number and/or concentration of couplexes and binding agents in step h. in the second partition.

2. The method according to claim 1, wherein determining the binding characteristics in step b. comprises determining for each binding agent of a first and a of a second type: the dissociation constant from the target analyte, the specificity of the binding to the target analyte, the labeled ratio, the label synthesis error, and/or the optimal concentration (number of molecules per unit volume) required for binding to the target analyte.

3. The method according to claim 1, wherein the binding agent of a first type and the binding agent of a second type, each comprise a nucleic acid label that is unique for the type of binding agent and wherein the bi-component detection method in step h. comprises the steps of:
amplifying inside each of the second partitions the nucleic acid sequence labels, and/or
combining the plurality of second partitions and performing nucleic acid sequence analysis in order to determine the number and identity of nucleic acid sequence labels and thereby the number and/or concentration of couplexes.

4. The method according to claim 1, wherein the bi-component detection method in step h. comprises performing a linking step between the label of the binding agent of a first type and the binding agent of a second type in each second partition.

5. The method according to claim 1, wherein the linking step in step h. comprises linking inside each second partition the nucleic acid sequence label of the binding agent of a first type to the nucleic acid sequence label of the binding agent of a second type if both form a couplex with the first target analyte.

6. The method according to claim 1, wherein the cells in the sample are permeabilized for the binding agent of a first type and the binding agent of a second type before bringing the binding agent of a first type and the binding agent of a second type into contact with the sample, such that one or more of the binding agent of a first type and the binding agent of a second type enters into one or more of the cells after having been brought into contact with the sample in step d.

7. The method according to claim 1, wherein the binding agent of a first type and the binding agent of a second type, each comprise a nucleic acid label that is unique for the type of binding agent, and
wherein the plurality of first partitions in step f. are further complemented with PCR-reagents and nucleic acid amplification primers comprising a barcode, which is unique to each first partition and the single cell therein, and
wherein an additional step of nucleic acid amplification is performed after cell lysis within each first partition.

8. The method according to claim 1, wherein the binding agent of a first type and the binding agent of a second type, each comprise a nucleic acid label that is unique for the type of binding agent and wherein each nucleic acid sequence label comprises an identifier sequence (barcode) and a unique molecular identifier (UMI), unique for each label molecule.

9. The method according to claim 1, wherein the sample comprises RNA and wherein the plurality of first partitions in step f. are further complemented with reverse transcription reagents, and RNA is converted into cDNA concurrently during any one or all of steps d-g, and wherein, if nucleic acid sequence analysis is performed in step h., said analysis comprises Next Generation Sequencing (NGS) or qPCR analysis of the cDNA generated in steps d-g.

10. The method according to claim 1, wherein the binding agent of a first type and the binding agent of a second type, each comprising a nucleic acid label that is unique for the type of binding agent and wherein the amplification in step h. involves the performance of PCR and/or droplet PCR and/or digital PCR and/or real-time PCR and/or RT-PCR.

11. The method according to claim 1, wherein the at least one target analyte comprises at least a first and a second target analyte, and wherein in step a. for each of the at least first and second target analytes at least a specific binding agent of a first type and a specific binding agent of a second type are provided.

12. The method according to claim 11, wherein
if the number of different target analytes is less than 5, and, if a nucleic acid sequence analysis is performed in step h., said analysis comprises digital PCR and/or real-time PCR, or
if the number of different target analytes is more than or equal to 5, and, if a nucleic acid sequence analysis is performed in step h., said analysis comprises Next Generation Sequencing (NGS).

13. The method according to claim 1, wherein the binding agent is selected from the group consisting of comprising antibodies, engineered protein scaffolds and aptamers.

14. The method according to claim 13, wherein the target analyte is selected from the group consisting of comprising peptides, proteins, chemically modified peptides or proteins, nucleic acids, chemically modified nucleic acids and any complexes thereof.

15. The method according to claim 1, wherein the sample is provided in step c. after being processed by a method selected from the group consisting of comprising fluorescence activated cell sorting (FACS), magnetic-activated cell sorting (MACS), capture laser microdissection (LCM), manual cell picking/micromanipulation, microfluidic separation and on-demand optically intercepted and controlled single-cell printing.

16. The method according to claim 7, wherein the plurality of first partitions in step f. are further complemented by reverse transcription-reagents.

17. The method according to claim 7, wherein the plurality of first partitions in step f. are further complemented by a protein-cross-linking agent and/or proteinase inhibitors and/or nuclease inhibitors.
